(19) United States
(12) Patent Application Publication
(10) Pub. No.: US 2023/0321063 A1
(43) Pub. Date: Oct. 12, 2023

(54) COMPOSITIONS AND METHODS TO REDUCE NEUROINFLAMMATION

(71) Applicant: Seattle Children's Hospital d/b/a Seattle Children's Research Institute, Seattle, WA (US)

(72) Inventors: Simon C. Johnson, Snohomish, WA (US); Julia Claire Stokes, Bellevue, WA (US); Rebecca Lois Bornstein, Seatte, WA (US); Margaret Mary Sedensky, Seattle, WA (US); Philip G. Morgan, Seattle, WA (US); Russell P. Saneto, Brier, WA (US)

(73) Assignee: Seattle Children's Hospital d/b/a Seattle Children's Research Institute, Seattle, WA (US)

(21) Appl. No.: 18/042,444

(22) PCT Filed: Aug. 20, 2021
(86) PCT No.: PCT/US21/47023

§ 371 Date: Feb. 21, 2023

Related U.S. Application Data
(60) Provisional application No. 63/068,312, filed on Aug. 20, 2020.

(51) Int. Cl. A61K 31/444; A61K 45/06; A61K 31/519; A61K 38/13; A61K 31/436; A61P 25/28

(52) U.S. Cl. CPC A61K 31/444; A61K 45/06; A61K 31/519; A61K 38/13; A61K 31/436; A61P 25/28

(57) ABSTRACT

The use of inhibitors upstream of mTOR in the CSF1 pathway of neuroinflammation, inhibitors of chemokine receptor CXCR3, functional derivatives thereof, and/or immunosuppressant drugs to reduce neuroinflammation are disclosed. The inhibitors and/or immunosuppressant drugs can treat genetic or environmental encephalopathies and/or reduce microglial activation. Treated encephalopathies include Leigh Syndrome and Wernicke encephalopathy.

### CROSS-REFERENCE TO RELATED APPLICATION

This application is a U.S. National Phase Patent Application based on International Patent Application No. PCT/US2021/047023, filed on Aug. 20, 2021, which claims priority to U.S. Provisional Patent Application No. 63/068,312 filed on Aug. 20, 2020, each of which is incorporated herein by reference in its entirety as if fully set forth herein.

### STATEMENT REGARDING FEDERALLY SPONSORED RESEARCH OR DEVELOPMENT

This invention was made with government support under grant GM126147 awarded by the National Institutes of Health. The government has certain rights in the invention.

### FIELD OF THE DISCLOSURE

The current disclosure provides use of inhibitors and/or immunosuppressant drugs to reduce neuroinflammation. The inhibitors include inhibitors upstream of mechanistic target of rapamycin (mTOR) in the CSF1 pathway of neuroinflammation. The inhibitors further include inhibitors of chemokine receptor CXCR3. Immunosuppressant drugs include prednisolone and dexamethasone. The inhibitors and/or immunosuppressant drugs can be used to treat genetic or environmental encephalopathies and/or to reduce glial cell activation. Treated encephalopathies include Leigh Syndrome and Wernicke encephalopathy.

### BACKGROUND OF THE DISCLOSURE

Encephalopathy is a term for any diffuse disease of the brain that alters brain function or structure. Encephalopathy may be caused by infectious agents (e.g., bacteria, virus, or prion), metabolic or mitochondrial dysfunction, brain tumor or increased pressure in the skull, prolonged exposure to toxic elements, chronic progressive trauma, poor nutrition, or lack of oxygen or blood flow to the brain. Depending on the type and severity of encephalopathy, common neurological symptoms include progressive loss of memory and cognitive ability, subtle personality changes, inability to concentrate, lethargy, and progressive loss of consciousness. Other neurological symptoms include myoclonus (involuntary twitching of a muscle or group of muscles), nystagmus (rapid, involuntary eye movement), tremor, muscle atrophy and weakness, dementia, seizures, and loss of ability to swallow or speak.

Mitochondrial encephalopathy is a severe clinical presentation of genetic mitochondrial disease which impacts infants and children and has no effective clinical therapy. A hallmark of mitochondrial encephalopathy is formation of symmetric, progressive, necrotizing lesions in specific areas of the brain, including the brainstem and cerebellum. These lesions accumulate astrocytes, which are support cells for neurons, and microglia, which are considered the white blood cells of the brain and are associated with a loss of neuron mass.

Leigh Syndrome, also known as subacute necrotizing encephalopathy, is a serious disease characterized by psychomotor retardation, seizures, hypotonia and weakness, ataxia, eye abnormalities including vision loss, difficulty in swallowing, and lactic acidosis. The disease can result in lesions to or degeneration of the basal ganglia, thalamus, brain stem, and spinal cord. A disease termed “Leigh-like Syndrome” is also recognized, which is characterized by neurologic abnormalities atypical for but suggestive of Leigh Syndrome. Leigh Syndrome is the most common mitochondrial disease of infancy.

Patients with Leigh Syndrome typically die before the age of five years, often from respiratory failure. Some patients with less severe disease may live to six or seven years, or even into their teen or adult years. Current treatments include thiamine (Vitamin B1), Coenzyme Q, or L-carnitine and oral sodium bicarbonate or sodium citrate to manage lactic acidosis. Unfortunately, these treatments are not particularly effective, and the prognosis for patients with Leigh Syndrome is extremely poor.

### SUMMARY OF THE DISCLOSURE

The current disclosure provides use of inhibitors and/or immunosuppressant drugs to reduce neuroinflammation. The inhibitors include inhibitors upstream of mechanistic target of rapamycin (mTOR) in the CSF1 pathway of neuroinflammation and/or functional derivatives thereof to reduce neuroinflammation. The inhibitors further include inhibitors of chemokine receptor CXCR3. Immunosuppressant drugs include prednisolone and dexamethasone.

Immunosuppressant drugs include prednisolone and dexamethasone. The inhibitors and/or immunosuppressant drugs can be used to treat genetic or environmental encephalopathies and/or to reduce glial cell activation. Treated encephalopathies include Leigh Syndrome and Wernicke encephalopathy. Treatment with inhibitors of the disclosure can: reduce leukocyte proliferation, reduce neurologic symptoms, improve respiratory function, reduce frequency of seizures, reduce cachexia, reduce hypoglycemia, reduce hyperlactemia, and/or reduce sensitivity to volatile anesthetics.

Inhibitors upstream of mTOR in the CSF1 pathway of neuroinflammation bind the CSF1R receptor and reduce binding by the natural CSF-1 ligand and/or inhibit the P110γ or P110δ microglia specific catalytic subunits of phosphatidylinositol-3-kinase (PI3K). Examples of inhibitors upstream of mTOR in the CSF1 pathway of neuroinflammation include Pexidartinib, PLX 5622, and IPI-549, and/or functional derivatives thereof.

Inhibitors of chemokine receptor CXCR3 bind CXCR3 and reduce binding by a natural ligand of CXCR3. In particular embodiments, the natural ligand of CXCR3 includes interferon γ-inducible 10 kD Protein (IP-10). Examples of inhibitors of chemokine receptor CXCR3 include: AMG487, TAK-779, SCH 546738, NBI-74330, PS372424, and/or functional derivatives thereof.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIG. 1. An inhibitor inhibits a specific glial cell activation pathway where the signaling factor colony stimulating factor 1 (CSF-1) activates intracellular signaling (involving the mechanistic target of rapamycin (mTOR) kinase) through the CSF1 receptor (CSF-1R). The inhibitor particularly blocks the extracellular binding of CSF-1 to CSF1R, reducing mTOR signaling and associated glial cell proliferation and activation. Class 1 Phosphoinositide 3-kinase (PI3K) microglia specific catalytic subunit enzymes (Phosphatidylinositol-4,5-bisphosphate 3-kinase catalytic subunit gamma isoform (P110γ) and Phosphatidylinositol-4,5-bisphosphate 3-kinase catalytic subunit delta isoform (P1106)) are inhibited intracellularly by P110γ and P110δ inhibitors, reducing mTOR signaling and associated glial cell proliferation and activation. mTOR inhibitors block mTOR kinase intracellularly, reducing mTOR signaling and associated glial cell proliferation and activation.

FIGS. 2. 100, 200, and 300 mg/kg/day Pexidartinib affect neurologic disease onset and severity as measured by clasping—a mild early sign of neurologic dysfunction.

FIGS. 3. 100, 200, and 300 mg/kg/day Pexidartinib provide a dose-dependent effect in neurologic disease onset and severity as measured by Rotarod performance—a general indicator of overall health status. The utilized paradigm (a steady slow pace latency to fall up to 10 min) is optimized for this very sick model.

FIGS. 4A-4L. Isoform specific inhibition of PI3K catalytic subunit p110γ, but not p110α, p110β, or p110δ, significantly attenuates disease in the Ndufs4(KO) mouse model of LS. (FIG. 4A) Disease course in untreated Ndufs4(KO) animals. Mortality, onset of cachexia, and onset of tractable behavioral symptoms related to neurologic dysfunction are shown. Disease symptoms on or onset shortly after postnatal day 37, with a rapid progression of symptoms until death by a median age of P63 (in this study). Data here, and in subsequent graphs, are shown as ‘percent ever’—when animals display a given symptom (see Experimental Methods in Example 1) they are scored and scoring for any given animal is not reversed even if the symptom is not observed on later dates. (FIG. 4B) Survival curves and associated lifespan and dosage data for Ndufs4(KO) animals treated with isoform specific inhibitors of the PI3K catalytic subunits p110α, p110β, p110δ, or p110γ, or control chow. Published rapamycin treatment data is overlayed for reference (lighter grey). Grey dashed line indicates median lifespan of rapamycin treated Ndufs4(KO) animals. A key for PI3K catalytic subunit inhibitor treatment lifespan curves with PPM in chow, approximate oral dosing, and median lifespans is shown in the table. Published rapamycin data is provided for reference. *p<0.05, **p<0.005, and ***p<0.0005 by log-rank test. (FIGS. 4C-4E) Onset of clasping (FIG. 4C), ataxia (FIG. 4D), and circling (FIG. 4E) in Ndufs4(KO) mice treated with inhibitors of PI3K catalytic subunits p110α, p110β, p110δ, or p110γ (treatment groups noted with Greek symbols above curves). Mice are scored when the symptom presents on at least two consecutive days. *p<0.05, **p<0.005, ***p<0.0005, and ****p<0.0001 by log-rank test vs untreated Ndufs4(KO) animals. (FIG. 4F) Performance of control and catalytic subunit specific inhibitor treated Ndufs4(KO) mice on a rotarod assay. *p<0.05, **p<0.005, ***p<0.0005, and ****p<0.0001 by unpaired, unequal variances (Welch's), t-test. (FIG. 4G) Scatter plots of Ndufs4(KO) mouse weight as a function of age and treatment, with local regression (Lowess) curve overlayed to display population trends. control, BYL719 (p110α inhibitor), GSK2636771 (p110β inhibitor), and CAL-101 (p110δ) treated Ndufs4(KO) mice are indicated on the left graph. ABI-009 (mTOR inhibitor) and IPI-549 (p110γ inhibitor) treated Ndufs4(KO) mice are indicated on the right graph. (FIG. 4H) Onset of cachexia in Ndufs4(KO) mice treated with inhibitors of PI3K catalytic subunits p110α, p110β, p110δ, or p110γ (treatment groups noted with Greek symbols above curves). Cachexia onset, scored after completion of the survival studies, was scored as the day of life when an individual animal began showing weight-loss after P37 (see Experimental Methods in Example 1). *p<0.05, **p<0.005, ***p<0.0005, and ****p<0.0001 by log-rank test vs untreated Ndufs4(KO) animals. (FIG. 4I) Blood glucose by age in control and PI3K catalytic subunit inhibitor treated Ndufs4(KO) animals. Each point represents the median value measured for one animal during the given time period (datapoints are biological replicates). *p<0.05, **p<0.005, ***p<0.0005, and ****p<0.0001 by unpaired, unequal variances (Welch's) t-test. (FIG. 4J) Growth rate during the P21-P35 period of rapid post-natal growth. Datapoints represent individual animals. *p<0.05, **p<0.005, ***p<0.0005, and ****p<0.0001 by unpaired, unequal variances (Welch's) t-test. (FIG. 4K) Maximum animal weight over the course of lifespan. *p<0.05, **p<0.005, ***p<0.0005, and ****p<0.0001 by unpaired, unequal variances (Welch's) t-test. (FIG. 4L) Cause of death in survival studies by treatment group (see Experimental Methods in Example 1). For all panels, error bars represent SEM.

FIGS. 5A-5L. Leukocyte depletion prevents central nervous system (CNS) lesions and significantly attenuates disease in the Ndufs4(KO) model of LS. (FIG. 5A) Dose-dependent impact of the CSF1R inhibitor pexidartinib (top graph) and mTOR inhibitor rapamycin (ABI-009) (bottom graph) on the fraction of microglia (brain resident leukocytes) in mixed primary brain cultures (see Experimental Methods in Example 1). Error bars represent SEM, dashed lines show the 95% confidence interval for an [Inhibitor] vs. response (three parameters) least squares fit. Representative images of mixed primary brain cultures stained with an anti-Iba1 antibody (green, microglia) and DAPI (blue, nuclei). (FIG. 5B) Representative pictures of control and Ndufs4(KO) animals treated with control diet or 300 mg/kg/day pexidartinib via chow. Pexidartinib treatment caused animal fur to whiten. (FIGS. 5C, 5D) Quantification of brainstem (FIG. 5C) and cerebellar peduncle (FIG. 5D) lesion size (area of lesion in central slice in serial sectioning, see Experimental Methods in Example 1) in control and 300 mg/kg/d pexidartinib treated control and Ndufs4(KO) animals. Quantification (left graph) and representative images (right images) for both regions. Representative images are only provided for Ndufs4(KO) animals as control mice do not develop lesions (see FIGS. 6A, 6B for quantification of microglia and astrocyte cell numbers in control and Ndufs4(KO) mice). Microglia are stained antibodies targeting Iba1 (red), astrocytes are stained with GFAP targeting antibodies (green), and nuclei stained with DAPI (blue) (see Experimental Methods in Example 1). Lesion areas indicated by dashed white lines. Ages are noted in representative images (P78, etc). ****p<0.0001 by unpaired, unequal variances (Welch's), t-test. (FIGS. 5E-5G) Onset of clasping (FIG. 5E), ataxia (FIG. 5F), and circling (FIG. 5G) in Ndufs4(KO) mice fed control diet (‘untreated’, black lines) or administered pexidartinib at 100, 200, or 300 mg/kg/day Ndufs4(KO) mice. *p<0.05, **p<0.005, ***p<0.0005, and ****p<0.0001 by log-rank test vs untreated Ndufs4(KO) animals. (FIG. 5H) Performance of control and pexidartinib treated animals on a rotarod assay. *p<0.05, **p<0.005, and ****p<0.0001 by unpaired, unequal variances (Welch's), t-test. (FIG. 5I) Representative traces of breathing activity in control and Ndufs4(KO) mice fed control chow or administered 300 mg/kg/d pexidartinib. (FIG. 5J) Multivariable plotting of respiratory amplitude irregularity, frequency, and frequency irregularity in control and Ndufs4(KO) mice fed control chow or administered 300 mg/kg/d pexidartinib. (FIG. 5K) Single variable analysis of data in FIG. 5J. Datapoints represent individual animals, error bars show SEM. *p<0.05, **p<0.005, and ****p<0.0001 by unpaired, unequal variances (Welch's), t-test. (FIG. 5L) Respiratory responses to increased environmental CO₂. Pairwise data shown for responses in individual mice **p<0.005 by Wilcoxon matched-pairs signed rank test.

FIGS. 6A-6I. Leukocyte depletion prevents microgliosis and astrocytosis throughout the brain and rescues a range of systemic symptoms associated with LS in the Ndufs4(KO) mice. (FIG. 6A) Quantification of microglia (left graph) and astrocytes (right graph) in the cortex of control and 300 mg/kg/d pexidartinib treated control and Ndufs4(KO) (see Experimental Methods in Example 1). Representative images of cortex. Datapoints represent individual animals, error bars show SEM. *p<0.05 and **p<0.005 by unpaired, unequal variances (Welch's), t-test. (FIG. 6B) Quantification of microglia (left graph) and astrocytes (right graph) in brainstem regions outside of overt lesions in control and 300 mg/kg/d pexidartinib treated control and Ndufs4(KO). See FIGS. 5C, 5D for representative images. Datapoints represent individual animals, error bars show SEM. *p<0.05 and **p<0.005 by unpaired, unequal variances (Welch's), t-test. (FIG. 6C) Frequency of seizures at P30 in the rotarod assay by treatment (Ndufs4(KO) genotype only—seizures not observed in control mice). *p<0.05 by Fisher's exact test. (FIG. 6D) Time to seizure for animals in Ndufs4(KO) mice in the rotarod assay at P30. All datapoints shown (none censored). *p<0.05 by log-rank test. (FIG. 6E) Weight (left graph) and cachexia (right graph). Black line—control treated, and lines for 100 mg/kg/d, 200 mg/kg/d, and 300 mg/kg/d treated Ndufs4(KO) animals are indicated. (Weight, left graph) Scatter plots of Ndufs4(KO) mouse weight as a function of age and treatment, with local regression (Lowess) curve overlayed to display population trends. (Cachexia, right graph) Cachexia onset (see FIG. 4H and Experimental Methods in Example 1) in control treated. (FIG. 6F) Blood glucose by age in control and pexidartinib treated Ndufs4(KO) animals. Each point represents the median value measured for one animal during the given time period (datapoints are biological replicates). *p<0.05 and ****p<0.0001 by unpaired, unequal variances (Welch's) t-test. (FIG. 6G) Blood lactate levels in response to a glucose bolus (2 g/kg) in control and Ndufs4(KO) mice at pre-disease (P25) and early disease (P45) ages. (left graph) Time-course of lactate levels and (right graph) total area under the curve (AUC) for blood lactate from 0-90 min. Error bars represent SEM, **p<0.005 by unpaired, unequal variances (Welch's) t-test. (FIG. 6H) Blood lactate levels in response to a glucose bolus (2 g/kg) in untreated control and Ndufs4(KO) mice and Ndufs4(KO) mice treated with pexidartinib (300 mg/kg/d in chow), IPI-549 (100 mg/kg/d in chow), or rapamycin (ABI-009 formulation, 8 mg/kg/d by IP injection). Time-course of lactate levels and total area under the curve (AUC) for blood lactate from 0-90 min. Error bars represent SEM, *p<0.05, **p<0.005, ****p<0.0001 by unpaired, unequal variances (Welch's) t-test. (FIG. 6I) (left graph) Change in blood lactate concentration in control and Ndufs4(KO) mice after a 30 min exposure to 0.4% isoflurane and the impact of treatment with 300 mg/kg/d pexidartinib. *p<0.05, **p<0.005 by unpaired, unequal variances (Welch's) t-test. (right graph) Mean alveolar concentration (MAC) of isoflurane associated with anesthesia in control and 300 mg/kg/d pexidartinib treated Ndufs4(KO) mice. **p<0.005 by unpaired, unequal variances (Welch's) t-test.

FIGS. 7A-7D. Ndufs4(KO) survival is dose-dependently extended by pexidartinib such that survival appears limited by drug toxicity rather than CNS disease. (FIG. 7A) Survival and cause of death in Ndufs4(KO) mice treated with increasing doses of pexidartinib. Survival curves. Black line—control treated (‘control chow’) Ndufs4(KO). Survival curves for Ndufs4(KO) mice treated with 100, 200, or 300 mg/kg/d pexidartinib are indicated. Survival curves for control animals treated with 300 mg/kg/d pexidartinib and animals treated with rapamycin are indicated (for reference). The table shows median lifespans and dosing data associated with survival curves. Cause of death for Ndufs4(KO) animals in control and pexidartinib treatment groups is indicated (all control animals on pexidartinib 300 mg/kg/d died of unknown causes with no evident illness). (FIG. 7B) Plasma ALT (left graph) and AST (right graph) levels as determined by enzymatic activity assay. *p<0.05 and **p<0.005 by unpaired, unequal variances (Welch's) t-test. (FIG. 7C) Concentrations of select chemokines in brainstem of control and Ndufs4(KO) animals at P25 and P45. (upper left-hand graph) Leukemia inhibitory factor (LIF), (upper right-hand graph) Interferon gamma-induced protein 10 (IP-10), (lower left-hand graph) Vascular endothelial growth factor (VEGF), and (lower right-hand graph) interferon γ (IFN γ). *p<0.05, **p<0.005, and ***p<0.0005 by unpaired, unequal variances (Welch's) t-test. (FIG. 7D) (left schematic) The generally accepted model for disease pathogenesis in LS. In this model, mitochondrial defects lead to CNS cell death through direct effects on energetics or ROS, driving tissue necrosis, which leads to inflammation. The pathogenesis of hyperlactemia has been thought to direct effects of mitochondrial dysfunction and not explored in the context of CNS damage. (right schematic) Without being bound by any one theory, the data disclosed herein, when considered in context of published data showing that LS symptoms arise from mitochondrial dysfunction in glutamatergic neurons, indicates that is not simply a response to damage, but instead may be causally upstream of CNS lesion formation but downstream of the primary causal cell type. Furthermore, without being bound by any one theory, inflammation in the CNS (including both lesions and widespread brain inflammation) may drive many or most of the systemic symptoms in LS, including hyperlactemia.

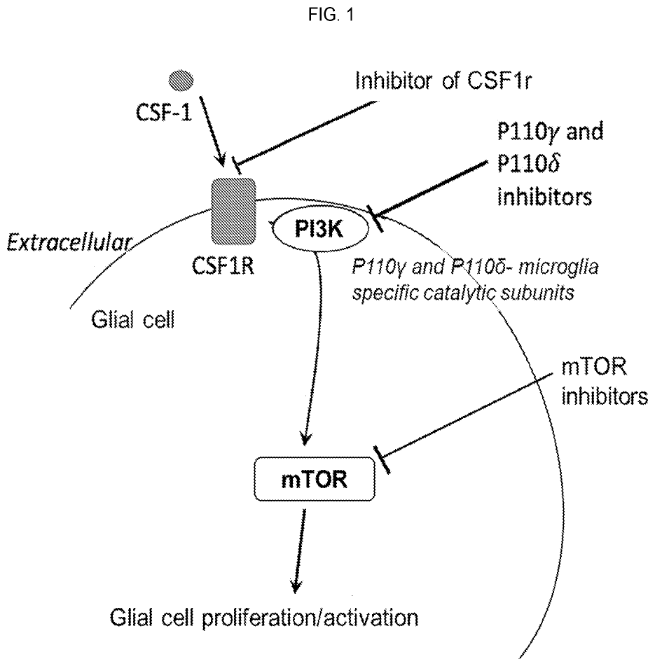

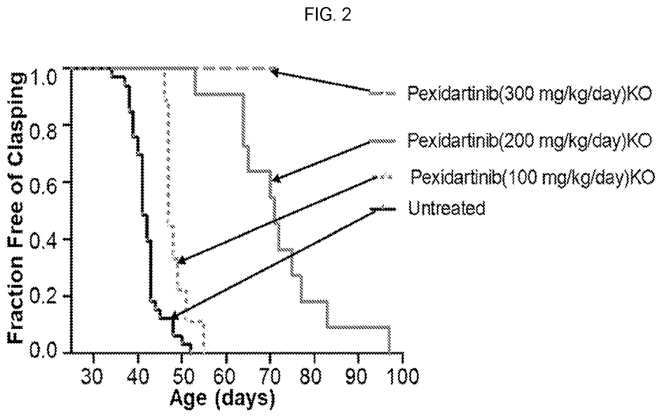

### DETAILED DESCRIPTION

Encephalopathy is a term for any diffuse disease of the brain that alters brain function or structure. Encephalopathy may be caused by infectious agents (e.g., bacteria, virus, or prion), metabolic or mitochondrial dysfunction, brain tumor or increased pressure in the skull, prolonged exposure to toxic elements (including solvents, drugs, radiation, paints, industrial chemicals, and certain metals), chronic progressive trauma, poor nutrition, or lack of oxygen or blood flow to the brain. Encephalopathies can be classified as environmental or genetic.

Mitochondrial encephalopathy is a severe clinical presentation of a genetic mitochondrial disease which impacts infants and children and has no effective clinical therapy. A hallmark of mitochondrial encephalopathy is formation of symmetric, progressive, necrotizing lesions in specific areas of the brain, including the brainstem and cerebellum. These lesions accumulate astrocytes, which are support cells for neurons, and microglia, which are considered the white blood cells of the brain and are associated with a loss of neuron mass.

Leigh Syndrome, also known as Leigh's disease and subacute necrotizing encephalopathy, is a serious disease characterized by multiple devastating symptoms, such as psychomotor retardation, seizures, hypotonia and weakness, ataxia, eye abnormalities including vision loss, difficulty in swallowing, and lactic acidosis. The disease can result in lesions to or degeneration of the basal ganglia, thalamus, brain stem, and spinal cord. See Leigh, D., “Subacute necrotizing encephalomyopathy in an infant,” J. Neurol. Neurosurg. Psychiat. 14:216-221 (1951). A disease termed “Leigh-like Syndrome” is also recognized, which is characterized by neurologic abnormalities atypical for but suggestive of Leigh Syndrome (Finsterer, J., “Leigh and Leigh-like syndrome in children and adults,” Pediatr. Neurol. 2008; 39:223-235). Criteria for diagnosis of Leigh syndrome include: (1) a neurodegenerative disease with variable symptoms, (2) caused by mitochondrial dysfunction from a hereditary genetic defect and (3) accompanied by bilateral central nervous system lesions. A genetic etiology is confirmed in 50% of patients, with more than 60 identified mutations in the nuclear and mitochondrial genomes. The incidence of Leigh Syndrome is estimated at 1 in 40,000 live births and is the most common mitochondrial disease of infancy.

Patients with Leigh Syndrome typically die before the age of five years, often from respiratory failure. Some patients with less severe disease may live to six or seven years, or even into their teen or adult years. Current treatments include thiamine (Vitamin B1), Coenzyme Q, or L-carnitine and oral sodium bicarbonate or sodium citrate to manage lactic acidosis. Unfortunately, these treatments are not particularly effective, and the prognosis for patients with Leigh Syndrome is extremely poor.

Metabolic encephalopathies are classified into two major categories. The first category arises due to the lack of glucose, oxygen, or metabolic cofactors. Conditions associated with this category include hypoglycemia, ischemia, hypoxia, hypercapnia, and vitamin deficiencies.

Wernicke encephalopathy is a neurological disorder of the brain, which results from a deficiency in thiamine (vitamin B1). It is considered an acute neuropsychiatric condition and requires immediate treatment to prevent the death of a subject. Symptoms of this disorder include mental confusion, vision problems, ataxia, delirium tremor, coma, hypothermia, and/or hypotension.

Korsakoff's syndrome has been described as a late neuropsychiatric manifestation of Wernicke encephalopathy and is considered to be the chronic phase of Wernicke encephalopathy. This disorder is usually associated with chronic alcohol use, dietary deficiencies, or systemic diseases such as AIDS or cancer. Symptoms of this disorder include visible cerebral lesions, loss of memory, confabulation, hallucinations, disorientation, and vision impairment.

The second category of metabolic encephalopathies result from peripheral organ dysfunction. Conditions associated with this category include hepatic encephalopathy, and uremic and dialysis encephalopathies. Symptoms of these encephalopathies include impairment of consciousness and cerebral function due to changes in brain chemistry at the neocortex and ascending reticular activating system of the brain. These impairments may result in diminished respiration, asterixis resulting in the loss of voluntary movement of muscles in the subject, and seizures.

Other metabolic disorders that give rise to metabolic encephalopathy include, disorders of fatty acid transport and beta-oxidation, disorders of organic acid metabolism, disorders of glycolysis, and disorders of urea cycle. The ingestion of toxic substances may also cause toxic-metabolic encephalopathy.

Acute encephalopathy is an acute brain dysfunction that typically occurs after a subject has been infected by a bacteria or virus. Acute necrotizing encephalopathy (ANE) is a parainfectious disease that is a form of acute encephalopathy. ANE is thought to be caused by environmental and host genetic factors. Most studies suggest that ANE occurs after a subject contracts a febrile illness caused by a viral infection, such as influenza or herpesvirus. An example of a host genetic factor that has been reported to contribute to the development of ANE includes a mutation in the Ran Binding Protein 2 (RANBP2) gene which may affect, among other things, mitochondrial energy production. Characteristics of a subject that has ANE include gastroenteritis, fever, seizures, erythema, organ failure, elevated levels of cytokines in the blood, and symmetric brain lesions on both the gray and white matter of the brain.

The current disclosure provides use of inhibitors and/or immunosuppressant drugs to reduce neuroinflammation. The inhibitors include inhibitors upstream of mechanistic target of rapamycin (mTOR) in the CSF1 pathway of neuroinflammation. The inhibitors further include inhibitors of chemokine receptor CXCR3. Immunosuppressant drugs include prednisolone and dexamethasone. Inhibitors upstream of mTOR in the CSF1 pathway of neuroinflammation bind the CSF1R receptor and reduce binding by the natural CSF-1 ligand and/or inhibit the P110γ or P110δ microglia specific catalytic subunits of PI3K. Inhibitors of chemokine receptor CXCR3 bind the CXCR3 receptor and reduce binding by a ligand of CXCR3.

The following aspects of the disclosure are now described in more detail: (i) Cytokines, cytokine receptors, and neuroinflammation; (ii) Colony stimulating factor 1 (CSF1) pathway and inhibitors of CSF1 receptor (CSF1R); (iii) Phosphatidyl inositol-3-kinase (PI3K) and inhibitors of PI3K catalytic subunits; (iv) Mechanistic target of rapamycin (mTOR); (v) Chemokine receptor CXCR3 and inhibitors of CXCR3; (vi) Immunosuppressant drugs; (vii) Compositions; (viii) Methods of Use; (ix) Kits; (x) Exemplary Embodiments; (xi) Experimental Examples; and (xii) Closing Paragraphs.

(i) Cytokines, cytokine receptors, and neuroinflammation. Cytokines are a class of small proteins that act as signaling factors, usually at picomolar or nanomolar concentrations, to regulate inflammation and modulate cell growth, survival, and differentiation. Cytokines can be pro-inflammatory or anti-inflammatory and are grouped into families based upon their structural homology or that of their receptors. Chemokines within the cytokine family function to induce cell migration (chemotaxis). Chemotactic cytokines are involved in leukocyte chemoattraction and trafficking of immune cells to locations throughout the body. Chemokines function in maintenance of homeostasis and induction of inflammation. Binding of a cytokine or chemokine ligand to its cognate receptor results in activation of the receptor and initiation of signaling events that regulate various cellular functions such as cell adhesion, phagocytosis, cytokine secretion, cell activation, cell proliferation, cell survival and cell death, apoptosis, angiogenesis, and proliferation.

Neuroinflammation refers to an inflammatory response within the brain or spinal cord. Neuroinflammation may be mediated by the production of cytokines, chemokines, reactive oxygen species (ROS), and/or secondary messengers (e.g., nitric oxide (NO), prostaglandins). These mediators are produced by resident central nervous system (CNS) glia (microglia and astrocytes), endothelial cells, and peripherally derived immune cells. Although transient, controlled inflammation may be beneficial to the CNS for injury-induced remodeling and training of innate immune cells for a neuro-protective phenotype, chronic, uncontrolled inflammation of the CNS may be detrimental. In particular embodiments, neuroinflammation is characterized by increased production of cytokines (e.g., IL-1β, IL-6, tumor necrosis factor alpha), chemokines (e.g., CCL2, CCL5, CXCL1), ROS, secondary messengers (e.g., NO, prostaglandins), inducible nitric oxide synthase, and/or other inflammatory mediators. In particular embodiments, the levels of IP-10, interferon α, leukemia inhibitory factor (LIF), or a combination thereof, are increased in a subject with neuroinflammation. In particular embodiments, neuroinflammation is characterized by gliosis, a process leading to scars in the CNS that involves the production of a dense fibrous network of neuroglia (supporting cells). In particular embodiments, gliosis can include microgliosis, an increase in the number of activated microglia at the site of a lesion in the CNS. In particular embodiments, gliosis can include astrocytosis, an increase in the number of activated astrocytes at the site of a lesion in the CNS. In particular embodiments, neuroinflammation is characterized by recruitment and trafficking of peripheral macrophages and neutrophils to a site of a lesion in the CNS. In particular embodiments, neuroinflammation is characterized by edema, increased blood brain barrier (BBB) permeability, and/or BBB breakdown. In particular embodiments, neuroinflammation can lead to vascular occlusion, ischemia, and/or cell death.

In particular embodiments, neuroinflammation is characterized by glial cell activation. In particular embodiments, glial cells can include microglia, long-lived cells in the brain and spinal cord that develop from myeloid precursor cells and function as macrophages in the CNS. In particular embodiments, glial cells can include astrocytes, star-shaped cells whose functions include axon guidance, synaptic support, control of blood brain barrier, and regulation of blood flow. In particular embodiments, glial cell activation includes: induction of expression and release of cytokines and chemokines from glial cells; proliferation of glial cells; alterations in morphology of glial cells; redistribution of cell-surface markers of glial cells (e.g., increased surface expression of ionized calcium binding adaptor molecule 1 (Iba1), glial fibrillary acidic protein (GFAP), CD68); migration of glial cells towards sites of injury or infection; increase in phagocytic efficiency of microglia; or a combination thereof. In particular embodiments, neuroinflammation is characterized by leukocyte proliferation. In particular embodiments, leukocytes include microglia.

(ii) Colony stimulating factor 1 (CSF1) pathway and inhibitors of CSF1 receptor (CSF1R). CSF1R (also known as FMS, FIM2, C-FMS, M-CSF receptor, and CD115) is a single-pass transmembrane receptor with an N-terminal extracellular domain and a C-terminal intracellular domain with tyrosine kinase activity. A human CSF1R amino acid sequence includes UniProt Accession ID P07333-1 encoded by NCBI reference sequence NM_001288705.3 (coding sequence includes nucleotide positions 128 to 3046). The ligands for CSF1R include CSF1 and IL-34. CSF1 is primarily expressed on neurons, microglia, astrocytes, and oligodendrocytes in the CNS. Human CSF1 amino acid sequences include: UniProt Accession ID P09603-1 encoded by NCBI reference sequence NM_000757.6 (coding sequence includes nucleotide positions 176 to 1840); UniProt Accession ID P09603-2 encoded by NCBI reference sequence NM_172210.3 (coding sequence includes nucleotide positions 176 to 1492); and UniProt Accession ID P09603-3 encoded by NCBI reference sequence NM_172211.4 (coding sequence includes nucleotide positions 176 to 946).

Ligand binding of CSF1 to CSF1R leads to receptor dimerization, upregulation of CSF1R protein tyrosine kinase activity, phosphorylation of CSF1R tyrosine residues, and downstream signaling events to stimulate monocyte survival, proliferation, and differentiation into macrophages and other monocytic cell lineages such as osteoclasts, dendritic cells, and microglia. Activation of CSF1R can lead to activation of: protein kinase C family members, MAP kinases, SRC family kinases, and/or the AKT1 (protein kinase B) signaling pathway, and release of pro-inflammatory chemokines. Activated CSF1R can transmit signals by proteins that directly interact with phosphorylated tyrosine residues in its intracellular domain, or by adapter proteins. Activated CSF1R promotes activation of STAT transcription factor family members STAT3, STAT5A and/or STAT5B. CSF1R signaling can be downregulated by protein phosphatases, including INPP5D/SHIP-1, that dephosphorylate the receptor and its downstream effectors, and by rapid internalization of the activated CSF1R. The data described herein reveal that the benefits of mTOR inhibition in a mouse model of Leigh syndrome result primarily from their effects on leukocyte, including microglia, proliferation. Furthermore, while PI3Kγ inhibition recapitulates mTOR inhibition, the data described herein demonstrate that direct pharmacologic targeting of leukocyte proliferation by inhibition of CSF1R significantly outperforms both PI3Kγ and mTOR inhibition.

Particular embodiments include use of Pexidartinib and/or functional derivatives thereof to treat genetic or environmental encephalopathies and/or to reduce glial cell activation. Treated encephalopathies include, for example, Leigh Syndrome and Wernicke encephalopathy.

Pexidartinib (also known as 1029044-16-3 or PLX3397) is a small molecule receptor tyrosine kinase inhibitor that targets the colony-stimulating factor-1 receptor (CSF1R), proto-oncogene receptor tyrosine kinase (c-Kit), and FMS-like tyrosine kinase 3 (FLT3). The molecular formula of Pexidartinib is C₂OH₁₅ClF₃N₅ and the IUPAC name of the structure is 5-[(5-chloro-1H-pyrrolo[2,3-b]pyridin-3-yl)methyl]-N-[[6-(trifluoromethyl)pyridin-3-yl]methyl]pyridin-2-amine. Pexidartinib has the following structure:

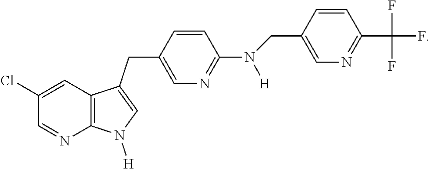 [SMILES: FC(F)(F)c1ccc(CNc2ccc(Cc3c[nH]c4ncc(Cl)cc34)cn2)cn1]

U.S. Ser. No. 10/123,998B2 discloses various functional derivatives of Pexidartinib including compounds having the structure of:

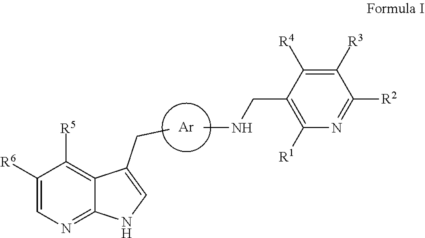

wherein:
Ar is selected from the group including:

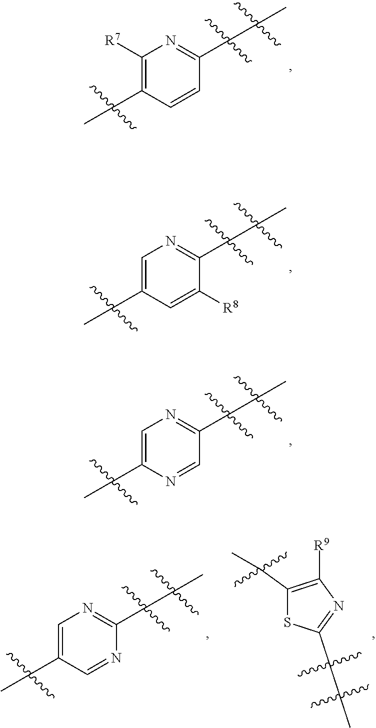

and
wherein

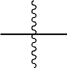

indicates the point of attachment of Ar —CH₂— of Formula I and where

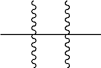 [SMILES: **]

indicates the point of attachment of Ar to —NH— of Formula I;
R¹, R², R³ and R⁴ are each independently selected from the group including —H, halogen, lower alkyl, halogen substituted lower alkyl, halogen substituted lower alkoxy, alkoxy substituted lower alkyl, cycloalkylamino, —CN, —O—R⁴⁰, —S(O)₂—R⁴¹, —S(O)₂—N(H)—R⁴², —N(H)—R⁴², —N(R⁴²)₂, and —N(H)—S(O)₂—R⁴³, provided that at least two of R¹, R², R³ and R⁴ are —H and one of R¹, R², R³ and R⁴ is other than hydrogen, wherein:
   R⁴⁹ is lower alkyl, fluoro substituted lower alkyl, methoxy substituted lower alkyl, or cycloalkyl; R⁴¹, R⁴² and R⁴³ are lower alkyl;
   R⁵ is selected from the group including —H, —F, —Cl, —Br, lower alkyl, halogen substituted alkyl, lower alkenyl, lower alkynyl, cycloalkyl, phenyl, pyrazolyl, —CN, —O—R¹⁰, —C(O)—N(H) —R¹¹, —C(O)—O—R¹¹, —S(O)₂—R¹², —S(O)₂—N(H)—R¹¹, —N(H)—C(O)—R¹², and —N(H)—S(O)₂—R¹², wherein pyrazolyl is optionally substituted with lower alkyl or heterocycloalkyl; R⁶ is selected from the group including H, halogen, lower alkyl, halogen substituted alkyl, lower alkenyl, lower alkynyl, cycloalkyl, phenyl, pyrazolyl, —CN, —O—R¹³, —C(O) —N(H)—R¹⁴, —C(O)—O—R¹⁴, —S(O)₂—R¹⁵, —S(O)₂—N(H)—R¹⁴, —N(H)—C(O)—R¹⁶, and —N(H)—S(O)₂—R¹⁵, wherein pyrazolyl is optionally substituted with lower alkyl or heterocycloalkyl;
R⁷ is H, halogen or lower alkyl;
R⁸ is H, halogen or lower alkoxy;
R⁹ is H or halogen;
R¹⁰ and R¹³ are independently —H, lower alkyl, lower alkyl substituted with —O—CH₃, lower alkyl substituted with di-alklylamine, or lower alkyl substituted with heterocycloalkyl;
R¹¹ and R¹⁴ are independently hydrogen or lower alkyl; and
R¹² and R¹⁵ are each independently lower alkyl.

Additional functional derivatives of Pexidartinib have the structure of:

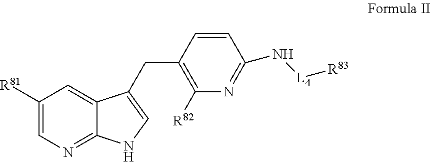

wherein:
L₄ is —CH₂CH₂—, —CH(R⁴⁰)—, —C(O)— or —C(O)NH—; R⁸¹ is selected from the group including hydrogen, —OR⁴¹, —CN, fluoro, chloro, lower alkyl, fluoro substituted lower alkyl, cycloalkyl, heterocycloalkyl, aryl and heteroaryl, wherein cycloalkyl, heterocycloalkyl, aryl or heteroaryl are optionally substituted with one or more substituents selected from the group including halogen, lower alkyl, fluoro substituted lower alkyl, —NHR⁴¹, —NR⁴¹R⁴¹, —OR⁴¹ and —S(O)₂R⁴¹; R⁸² is selected from the group including hydrogen, fluoro, C₁₋₃ alkyl, fluoro substituted C₂₋₃ alkyl, OH, C₁₋₃ alkoxy, and fluoro substituted C₁₋₃ alkoxy;
R⁸³ is heterocycloalkyl, heteroaryl, or

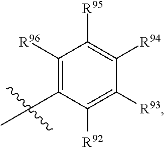

in which

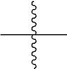

indicates the attachment point of R⁸³ to L₄ of Formula II, wherein heterocycloalkyl or heteroaryl are optionally substituted with one or more substituents selected from the group including halogen, lower alkyl, fluoro substituted lower alkyl, cycloalkylamino, —NHR⁴¹, —NR⁴¹R⁴¹, —OR⁴¹ and —S(O)₂R⁴¹;
   R⁹², R⁹³, R⁹⁴, R⁹⁵, and R⁹⁶ are independently selected from the group including hydrogen, halogen, lower alkyl, fluoro substituted lower alkyl, cycloalkylamino, —NHS(O)₂R⁴¹, —NHC(O)R⁴¹, —NHR⁴¹, —NR⁴¹R⁴¹, —OR⁴¹ and —S(O)₂R⁴¹;
   R⁴° is selected from the group including lower alkyl, and fluoro substituted lower alkyl;
   R⁴¹ at each occurrence is independently selected from the group including lower alkyl, cycloalkyl, heterocycloalkyl, aryl and heteroaryl, wherein lower alkyl is optionally substituted with one or more substituents selected from the group including fluoro, lower alkoxy, fluoro substituted lower alkoxy, lower alkylthio, fluoro substituted lower alkylthio, mono-alkylamino, di-alkylamino, cycloalkyl, heterocycloalkyl, aryl, and heteroaryl, and wherein cycloalkyl, heterocycloalkyl, aryl, and heteroaryl as R⁴¹ or as substituents of lower alkyl are optionally substituted with one or more substituents selected from the group including —OH, —NH₂, —ON, —NO₂, —S(O)₂NH₂, —C(O)NH₂, —OR⁴², —SR⁴², —NHR⁴², —NR⁴²R⁴², —NR³⁹C(O)R⁴², —NR³⁹S(O)₂R⁴², —S(O)₂R⁴², halogen, lower alkyl, fluoro substituted lower alkyl, and cycloalkylamino; and

R⁴² at each occurrence is independently selected from the group including lower alkyl, heterocycloalkyl and heteroaryl, wherein lower alkyl is optionally substituted with one or more substituents selected from the group including fluoro, lower alkoxy, fluoro substituted lower alkoxy, lower alkylthio, fluoro substituted lower alkylthio, mono-alkylamino, di-alkylamino, and cycloalkylamino, and wherein heterocycloalkyl and heteroaryl are optionally substituted with one or more substituents selected from the group including halogen, —CN, lower alkyl, fluoro substituted lower alkyl, lower alkoxy and fluoro substituted lower alkoxy.

For additional information regarding Pexidartinib and functional derivatives thereof, see: U.S. Ser. No. 10/123,998B2, US20190031654A1, US20190031766A1, US20180186882A1, US20180030051A1, US20180222982A1, US20170306038A1, U.S. Pat. No. 9,550,768B2, U.S. Pat. No. 8,722,702B2, U.S. Pat. No. 8,404,700B2, WO/2020142422A1, WO/2020139828A1, WO/2020131627A1, WO/2020131674A1, and WO/2020/127839A1.

Additional examples of CSF-1R inhibitors include AB-530, also known as N-[4-[3-(5-tert-Butyl-1,2-oxazol-3-yl)ureido]phenyl]imidazo[2,1-b]benzothiazole-2-carboxamide (Daiichi Sankyo), AC-708 (Ambit Bioscience), AC-710 (Ambit Bioscience), AC-855 (Ambit Bioscience), ARRY-382 (Array BioPharma), AZ-683 (Astra-Zeneca), AZD-6495 (Astra Zenenca), BLZ-3495 (Novartis), BLZ-945 (Novartis), N-(4-[[(5-tert-Butyl-1,2-oxazol-3-yl)carbamoyl]amino]phenyl)-5-[(1,2,2,6,6-pentamethylpiperidin-4-yl)oxy]pyridine-2-carboxamide methanesulfonate (Daiichi Sankyo), N-(4-[[(5-tert-Butyl-1,2-oxazol-3-yl)carbamoyl]amino]phenyl)-5-[(1,2,2,6,6-pentamethylpiperidin-4-yl)oxy]pyridine-2-carboxamide methanesulfonate (Diaiichi Sankyo), CT 1578 (CTI BioPharma), CYT-645 (Gilead), DCC 2909 (Deciphera), DCC-3014 (Deciphera), DP-4577 (Deciphera), DP-5599 (Deciphera), DP-6261 (Deciphera), ENMD-981693 (EntreMed), FMS kinase inhibitors (AEgera), GT-79 (Gerinda Therapeutics), GW-2580 also known as 5-[3-Methoxy-4-(4-methoxybenzyloxy)benzyl]pyrimidine-2,4-diamine (GlaxoSmithKline), Ilorasertib (University of Chicago), Ki-20227 (Kyowa Hakko Kirin), Linifanib (AbbVie), Masitinib (AB Science), Pexidartinib (Plexxikon), PLX 5622 (Plexxikon), PLX FK1 (Plexxikon), PLX-7486 (Plexxikon), REDX-05182 (Redx Oncology), and 5-cyano-N-[2-(cyclohexen-1-yl)-4-[1-[2-(dimethylamino)acetyl]piperidin-4-yl]phenyl]-1H-imidazole-2-carboxamide.

In particular embodiments, an inhibitor of CSF1R includes PLX 5622 (Plexxikon; Spangenberg et al. Nature Communications 2019; 10:3758; Ali et al. Aging (Albany NY). 2020; 12(3):2101-2122; Lei et al. PNAS 2020; 117(38):23336-23338). The molecular formula of PLX 5622 is C₂₁H₁₉F₂N₅O and the IUPAC name of the structure is 5-fluoro-N-[6-fluoro-5-[(5-methyl-1H-pyrrolo[2,3-b]pyridin-3-yl)methyl]-2-pyridinyl]-2-methoxy-3-pyridinemethanamine. PLX 5622 has the following structure:

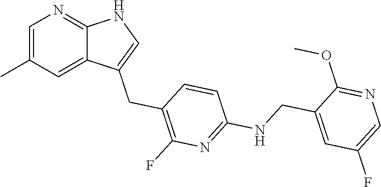 [SMILES: COc1ncc(F)cc1CNc1ccc(Cc2c[nH]c3ncc(C)cc23)c(F)n1]

Inhibitors of CSF1R can include antibodies or antibody binding fragments that interfere with binding of CSF1 to CSF1R. Anti-CSF1R antibodies are described in Sherr et al. Blood 1989; 73: 1786-1793; Ashmun et al. Blood 1989; 73:827-837; Kitaura et al. Journal of Dental Research 2008; 87:396-400; MacDonald et al. Blood 2010; 116(19):3955-3963; WO2009026303; WO2009112245; WO2011123381; WO2011140249; WO2013132044; WO2014036357; US20180346581; U.S. Pat. No. 9,765,147B2; and U.S. Pat. No. 9,192,667B2. Anti-CSF1R antibodies include emactuzumab (RG7155; Ries et al. Cancer Cell. 2014; 25(6):846-59) and MCS110.

Commercially available antibodies that bind CSF1R include: anti-mouse CSF1R rat monoclonal antibody clone AFS98 (ThermoFisher Scientific, Waltham, MA); anti-human CSF1R mouse monoclonal antibody clone 1486CT328.53.37 (ThermoFisher Scientific, Waltham, MA); anti-human CSF1R mouse monoclonal antibody clone 6B9B9 (ThermoFisher Scientific, Waltham, MA); anti-CSF1R rabbit polyclonal antibody (cat #PA5-25974; ThermoFisher Scientific, Waltham, MA); and anti-human CSF1R rat monoclonal antibody clone 12-3A3-1B10 (ThermoFisher Scientific, Waltham, MA).

(iii) Phosphatidylinositol-3-kinase (PI3K) and inhibitors of PI3K catalytic subunits. The PI3K/AKT intracellular signaling pathways regulate major cell processes such as cell growth, motility, survival, metabolism, and angiogenesis. PI3K refers to a group of plasma membrane-associated lipid kinases that can have three subunits: a p85 regulatory subunit, a p55 regulatory subunit, and a p110 catalytic subunit. PI3K can be divided into 3 classes, depending upon the structure of the kinase and specific substrates. Class 1 PI3K is a heterodimeric molecule composed of a regulatory subunit and can be further classified as class IA or class IB. Class IA PI3K includes p110α, p110β, and p110δ catalytic subunits encoded by genes PIK3CA, PIK3CB, and PIK3CD, respectively. The only catalytic subunit in Class IB is p110γ, encoded by PIK3CG. A human p110α (phosphatidylinositol 4,5-bisphosphate 3-kinase catalytic subunit α isoform) amino acid sequence includes UniProt Accession ID P42336 encoded by NCBI reference sequence NM_006218.4 (coding sequence includes nucleotide positions 324 to 3530). A human p110β (phosphatidylinositol 4,5-bisphosphate 3-kinase catalytic subunit β isoform) amino acid sequence includes UniProt Accession ID P42338 encoded by NCBI reference sequence NM_006219.3 (coding sequence includes nucleotide positions 356 to 3568). A human p110δ (phosphatidylinositol 4,5-bisphosphate 3-kinase catalytic subunit δ isoform) amino acid sequence includes UniProt Accession ID 000329 encoded by NCBI reference sequence NM_005026.5 (coding sequence includes nucleotide positions 210 to 3344). A human p110γ (phosphatidylinositol 4,5-bisphosphate 3-kinase catalytic subunit γ isoform) amino acid sequence includes UniProt Accession ID P48736 encoded by NCBI reference sequence NM_001282426.2 (coding sequence includes nucleotide positions 158 to 3466). Class II PI3K has three catalytic isoforms (C2α, C2β, and C2γ) but no regulatory subunits. Class III PI3K is a heterodimeric molecule composed of a catalytic Vps34 subunit and a regulatory Vps15/p150 subunit.

PI3K phosphorylates the 3′ position hydroxyl group of the inositol ring of phosphatidylinositol. PI3Ks can produce various 3-phosphorylated phosphoinositides, including phosphatidylinositol 3-phosphate, phosphatidylinositol (3,4)-bisphosphate, phosphatidylinositol (3,5)-bisphosphate, and phosphatidylinositol (3,4,5)-trisphosphate. In particular embodiments, PI3K converts phosphatidylinositol (4,5)-bisphosphate into phosphatidylinositol (3,4,5)-trisphosphate in vivo. The 3-phosphorylated phosphoinositides can bind and recruit signaling proteins (including kinases Akt/protein kinase B and PDK1) having phosphoinositide-binding domains to the cell membrane to activate cell growth and cell survival pathways. Phosphatase and tensin homologue deleted on chromosome 10 (PTEN) regulates the pathway by dephosphorylating phosphatidylinositol trisphosphate to phosphatidylinositol bisphosphate to prevent activation of downstream kinases.

Examples of Phosphatidylinositol 3-kinase-gamma (PI3K-γ or P110γ) inhibitors include Duvelisib, TG100-115, and IPI-549. Duvelisib (also known as IPI-145 or INK-1197) has a molecular formula of C₂₂H₁₇ClN₆O and IUPAC name of 8-chloro-2-phenyl-3-[(1S)-1-(7H-purin-6-ylamino)ethyl]isoquinolin-1-one. Duvelisib has the following structure:

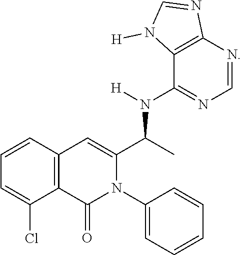 [SMILES: C[C@H](Nc1ncnc2nc[nH]c12)c1cc2cccc(Cl)c2c(=O)n1-c1ccccc1]

For additional information regarding Duvelisib and functional derivatives thereof, see US20190192541A1, US20190031766A1, US20190194212A1, and U.S. Pat. No. 9,216,982B2.

TG100-115 (also known as 6,7-Bis(3-hydroxyphenyl)pteridine-2,4-diamine) has a molecular formula of C₁₈H₁₄N₆O₂ and IUPAC name of 3-[2,4-diamino-7-(3-hydroxyphenyl)pteridin-6-yl]phenol. TG100115 has the following structure:

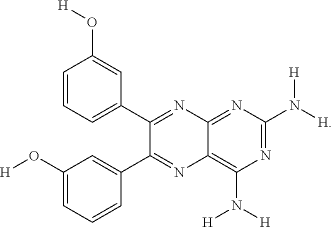

For additional information regarding TG100-11 and functional derivatives thereof, see US20190185477A1, US20170088553A1, U.S. Pat. No. 9,303,033B2, and US20120129849A1.

IPI-549 (also known as CID 91933883) has a molecular formula of C₃₀H₂₄N₈O₂ and IUPAC name of 2-amino-N-[(1S)-1-[8-[2-(1-methylpyrazol-4-yl)ethynyl]-1-oxo-2-phenylisoquinolin-3-yl]ethyl]pyrazolo[1,5-a]pyrimidine-3-carboxamide. IPI-549 has the following structure:

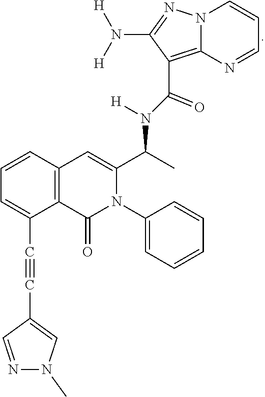 [SMILES: C[C@H](NC(=O)c1c(N)nn2cccnc12)c1cc2cccc(C#Cc3cnn(C)c3)c2c(=O)n1-c1ccccc1]

For additional information regarding IPI-549 and functional derivatives thereof, see US20190070166A1, US20190185477A1, and US20170088553A1.

An example of Phosphatidylinositol-4,5-bisphosphate 3-kinase catalytic subunit delta isoform (PI3Kδ or P110δ) inhibitors include Idelalisib, Fimepinostat, and Copanlisib. Idelalisib (also known as CAL-101) has a molecular formula of C₂₂H₁₈FN₇O and an IUPAC name of 5-fluoro-3-phenyl-2-[(1S)-1-(7H-purin-6-ylamino)propyl]quinazolin-4-one. Idelalisib has the following structure:

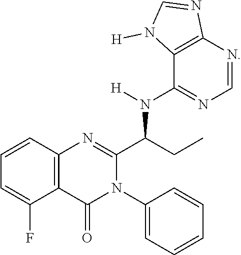 [SMILES: CC[C@H](Nc1ncnc2nc[nH]c12)c1nc2cccc(F)c2c(=O)n1-c1ccccc1]

For additional information regarding TG100-11 and functional derivatives thereof, see US20190144453A1, US20190192541A1, and U.S. Pat. No. 8,980,901B2.

Fimepinostat (also known as CUDC-907) has a molecular formula of C₂₃H₂₄N₈O₄S and an IUPAC name of N-hydroxy-2-[[2-(6-methoxypyridin-3-yl)-4-morpholin-4-ylthieno[3,2-d]pyrimidin-6-yl]methyl-methylamino]pyrimidine-5-carboxamide. Fimepinostat has the following structure:

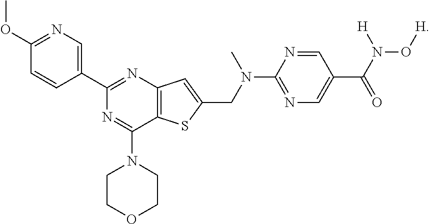

For additional information regarding Fimepinostat and functional derivatives thereof, see US20170362251A1, U.S. Pat. No. 8,906,909B2, and US20190076404A1.

Copanlisib (also known as BAY 80-6946) has a molecular formula of C₂₃H₂₈N₈O₄ and IUPAC name of 2-amino-N-[7-methoxy-8-(3-morpholin-4-ylpropoxy)-2,3-dihydro-1H-imidazo[1,2-c]quinazolin-5-ylidene]pyrimidine-5-carboxamide. Copanlisib has the following structure:

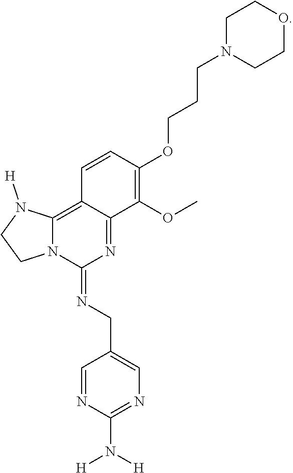 [SMILES: COc1c(OCCCN2CCOCC2)ccc2c3n(/c(=N/Cc4cnc(N)nc4)nc12)CCN3]

For additional information regarding Copanlisib and functional derivatives thereof, see US20190092775A1, U.S. Pat. No. 8,466,283B2, and U.S. Pat. No. 7,511,041B2.

Compounds used in the experimental work described in the Examples include the Phosphatidylinositol 3-kinase-alpha (PI3Kα) inhibitor BYL719 and the Phosphatidylinositol 3-kinase-beta (PI3Kβ) inhibitor GSK2636771.

BYL719 (also known as Alpelisib) has a molecular formula of C₁₉H₂₂F₃N₅O₂S and an IUPAC name of (2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide. BYL719 has the following structure:

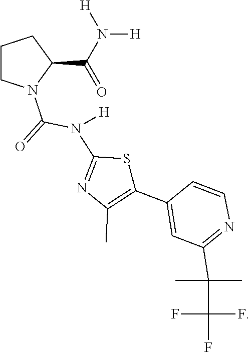 [SMILES: Cc1nc(NC(=O)N2CCC[C@H]2C(N)=O)sc1-c1ccnc(C(C)(C)C(F)(F)F)c1]

For additional information regarding BYL719 and functional derivatives thereof, see US20190119243A1, U.S. Ser. No. 10/208,011B2, and US20190185511A1.

GSKK2636771 (also known as 1372540-25-4) has a molecular formula of C₂₂H₂₂F₃N₃O₃ and an IUPAC name of 2-methyl-1-[[2-methyl-3-(trifluoromethyl)phenyl]methyl]-6-morpholin-4-ylbenzimidazole-4-carboxylic acid. GSK2636771 has the following structure:

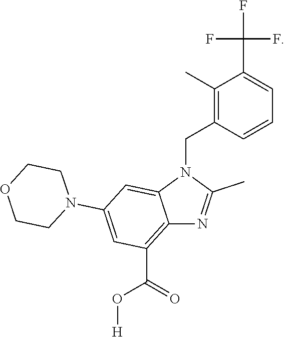 [SMILES: Cc1c(Cn2c(C)nc3c(C(=O)O)cc(N4CCOCC4)cc32)cccc1C(F)(F)F]

For additional information regarding GSK2636771 and functional derivatives thereof, see U.S. Pat. No. 8,541,411B2, U.S. Pat. No. 8,435,988B2, and US20190076404A1.

(iv) Mechanistic target of rapamycin (mTOR). mTOR is a serine/threonine kinase that is ubiquitously expressed in mammalian cells. mTOR integrates signals from nutrient intake, growth factors, and other cellular stimuli to regulate protein synthesis for cell growth, cell cycle progression, and cell metabolism. Downstream effectors of mTOR include 4EBP1 and P70S6 kinase. Positive regulators of mTOR include growth factors and their receptors (e.g., insulin-like growth factor 1 and its receptor IGFR-1; members of the human epidermal growth factor receptor and their associated ligands; and vascular endothelial growth factor receptors and their associated ligands). Negative regulators of mTOR include PTEN, tuberous sclerosis complex (TSC) 1 (hamartin), and TSC2 (tuberin). Signals to mTOR can be transmitted through PI3K-Akt. mTOR can exist as two distinct complexes: mTORC1 and mTORC2. mTORC1 is very sensitive to rapamycin and mTOR2 is less so. A human mTOR amino acid sequence includes UniProt Accession ID P42345 that can be encoded by NCBI reference sequence NM_004958.4 (coding sequence includes nucleotide positions 122 to 7771).

(v) Chemokine (C—X—C motif) receptor 3 (CXCR3, also known as CD183) is a receptor in the CXC chemokine receptor family of G protein coupled receptors. The CXCR3 receptor binds a number of ligands, such as monokine induced by interferon-γ (MIG; CXCL9), interferon γ-inducible 10 kD Protein (IP-10; CXCL10), Interferon γ-inducible T-cell α-Chemoattractant (I-TAC), and B cell-attracting chemokine-1 (BCA-1). Certain forms of CXCR3 also bind platelet factor-4 (PF-4; Lasagni et al. J. Exp. Med. 2003; 197: 1537-1549). There are at least two splice variants of CXCR3: (1) CXCR3-A (also called isoform 1), which can bind to chemokines MIG, IP-10, and I-TAC, and (2) CXCR3-B (also called isoform 2), which can bind to CXCL4, MIG, IP-10, and I-TAC. The expression of IP-10, MIG, and I-TAC is induced in tissues by interferons or tumor necrosis factor (TNF), potent mediators of inflammation. CXCR3A receptor is predominantly expressed on activated Th1 lymphocytes, but it is also present on natural killer cells, macrophages, dendritic cells, and B lymphocytes. CXCR3B receptor is expressed on endothelial cells and mediates angiostatic effects of MIG, IP-10, I-TAC, and PF-4. Human CXCR3 amino acid sequences include: UniProt Accession ID P49682-1 encoded by NCBI reference sequence NM_001504.2 (coding sequence includes nucleotide positions 63 to 1169); and UniProt Accession ID P49682-2 encoded by NCBI reference sequence NM_001142797.2 (coding sequence includes nucleotide positions 166 to 1413). A human IP-10 amino acid sequence includes UniProt Accession ID P02778 encoded by NCBI reference sequence NM_001565.4 (coding sequence includes nucleotide positions 67 to 363).

CXCR3 has been implicated as an important mediator of: inflammatory and immunoregulatory diseases and disorders including asthma and allergic diseases; autoimmune diseases such as rheumatoid arthritis and atherosclerosis; and tumor growth and metastasis. In leukocytes the binding of MIG, IP-10, or I-TAC, to CXCR3 can mediate chemotaxis. All three ligands can induce calcium flux and phosphorylation of ERK and AKT kinases. Without being bound by any one theory, MIG and IP-10 can activate STAT1/5 transcription factors to enforce transcription factor T box expressed in T cells (Tbet)/retinoic acid-related orphan receptor-γt (RORγT) transcription factor expression, whereas I-TAC activates STAT3/6 transcription factors to enforce GATA3 transcription factor expression (Groover et al. F1000Research 2020; 9:1197).

Inhibitors of CXCR3 are useful in compositions and methods of the present disclosure. An inhibitor of CXCR3 includes an agent capable of reducing, disrupting, or modulating the CXCR3 signaling in a cell. An inhibitor of CXCR3 includes an agent (e.g., an antibody or a small molecule) that interferes with the interaction between CXCR3 and a ligand thereof, such as CXCL4, MIG, IP-10, and/or I-TAC. In particular embodiments, an inhibitor of CXCR3 includes a CXCR3 agonist (e.g., PS372424) or a CXCR3 antagonist (e.g., TAK-779). In particular embodiments, the CXCR3 inhibitor includes an agent that suppresses CXCR3 transcription and/or translation, thereby reducing the mRNA/protein level of CXCR3 (e.g., an inhibitory polynucleotide or oligonucleotide such as small interfering RNA (siRNA), short hairpin RNA (shRNA), or an antisense oligonucleotide). In particular embodiments, the CXCR3 inhibitor includes a ribozyme that is complementary to a CXCR3 nucleic acid (e.g., a CXCR3 mRNA) and cleaves the CXCR3 nucleic acid. In particular embodiments, the CXCR3 inhibitor includes an antibody that specifically binds to CXCR3 and neutralizes its activity. The term “antibody” includes polyclonal, monoclonal, humanized, chimeric, Fab fragments, Fv fragments, F(ab′) fragments and F(ab′)₂ fragments, as well as single chain antibodies (scFv), fusion proteins, and other synthetic proteins which include the antigen-binding site of the antibody. Antibodies can be made by the skilled person using methods and commercially available services and kits known in the art.

In particular embodiments, the CXCR3 inhibitor includes a non-antibody peptide or protein, or a synthetic binding molecule. The non-antibody peptide or protein, or the synthetic binding molecule may interfere with the activity of CXCR3, such as by competing with a natural ligand for CXCR3, e.g., competing with IP-10. A natural ligand as described herein refers to a ligand produced endogenously by a cell that binds to the ligand's cognate receptor. For example, natural ligands that bind to CXCR3 include CXCL4, MIG, IP-10, I-TAC, and PF-4. Proteins and peptides may be designed using any method known in the art, e.g., by screening libraries of proteins or peptides for binding to CXCR3 or inhibition of CXCR3 binding to a ligand, such as IP-10. A synthetic binding molecule can include aptamers and synbodies. An aptamer is a nucleic acid that can form specific three dimensional structures exhibiting high affinity binding to a wide variety of cell surface molecules, proteins, and/or macromolecular structures. Aptamers are commonly identified by an in vitro method of selection sometimes referred to as Systematic Evolution of Ligands by EXponential enrichment or “SELEX”. SELEX typically begins with a very large pool of randomized polynucleotides which is generally narrowed to one aptamer ligand per molecular target. Synbodies are synthetic antibodies produced from libraries comprised of strings of random peptides screened for binding to target proteins of interest.

An inhibitor of CXCR3 as described herein may reduce the CXCR3 signaling in cells by at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more. The inhibitory activity of an inhibitor of CXCR3 can be determined by conventional methods, e.g., the CXCR3 bioassay method disclosed in US 20100305088, a binding assay (e.g., ligand binding or agonist binding), a signalling assay (e.g., activation of a mammalian G protein, induction of rapid and transient increase in the concentration of cytosolic free calcium), and/or cellular response function (e.g., stimulation of chemotaxis, exocytosis or inflammatory mediator release by leukocytes).

Inhibitors of CXCR3 useful in compositions and methods of the present disclosure include compounds having the structure of Formula III:

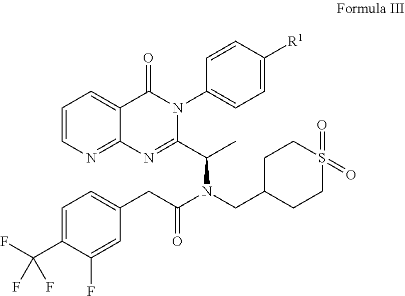

wherein R¹ is chloro or fluoro. Synthesis of compounds of Formula III are described in US20110034487A1.

In particular embodiments, inhibitors of CXCR3 useful in compositions and methods of the present disclosure include AMG487 (Walser et al. Cancer Res. 2006; 66:7701; US20110034487A1). The molecular formula of AMG487 is C₃₂H₂₈F₃N₅O₄ and the IUPAC name is N-[(1R)-1-[3-(4-ethoxyphenyl)-4-oxopyrido[2,3-d]pyrimidin-2-yl]ethyl]-N-(pyridin-3-ylmethyl)-2-[4-(trifluoromethoxy)phenyl]acetamide. AMG487 has the following structure:

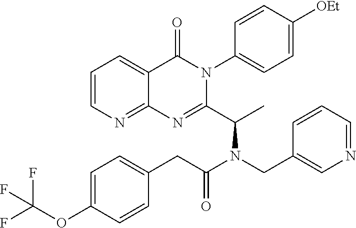 [SMILES: CCOc1ccc(-n2c([C@@H](C)N(Cc3cccnc3)C(=O)Cc3ccc(OC(F)(F)F)cc3)nc3ncccc3c2=O)cc1]

In particular embodiments, other inhibitors of CXCR3 useful in compositions and methods of the present disclosure include TAK-779 (IUPAC name: N,N-dimethyl-N-(4-[[[2-(4-methylphenyl)-6,7-dihydro-5H-benzocyclohepten-8-yl]carbon-yl]amino]benzyl)-tetrahydro-2H-pyran-4-aminium chloride; Yang et al. Eur. J. Immunol. 2002; 32:2124; Gao et al. J. Leukoc. Biol. 2003; 73:273; Ni et al. British Journal of Pharmacology 2009; 158: 2046-2056); SCH 546738 (Jenh et al. BMC Immunology 2012; 13:2); NBI-74330 (IUPAC name: N-1R-[3-(4-ethoxy-phenyl)-4-oxo-3,4-dihydro-pyrido[2,3-d]pyrimidin-2-yl]-ethyl-N-pyridin-3-ylmethyl-2-(4-fluoro-3-trifluoromethyl-phenyl)-acetamide; Heise et al. J Pharmacol Exp Ther. 2005; 313(3):1263-1271; Jopling et al. Br J Pharmacol. 2007; 152(8):1260-1271; van Wanrooij et al. Arterioscler Thromb Vasc Biol. 2008; 28(2):251-7); PS372424 (a 3-amino acid fragment of IP-10 (CXCL10); Stroke et al. Biochem Biophys Res Commun. 2006; 349(1):221-228; Boyle et al. PNAS 2012; 109(12):4598-4603; Boye et al. Nature Communications 2017; 8:1571); inhibitors described in US20070021611A1, US20070054919A1, US20070082913A1, WO2008008453A1, and WO2007109238A1 having a (piperidin-4-ylpiperazin-1yl) aromatic moiety core structure; and inhibitors described in U.S. Pat. No. 8,268,828B2, AU2005219322B2, US20180022810A1, U.S. Ser. No. 10/668,148B2, JP2005530813A, JP4955040B2, US20150202266A1, and WO2014018018A1.

Anti-CXCR3 antibodies are commercially available and include: anti-mouse CXCR3 monoclonal antibody clone CXCR3-173 (BioLegend, San Diego, CA); anti-human CXCR3 rabbit polyclonal antibody (cat #PA5-28741, ThermoFisher Scientific, Waltham, MA); anti-human CXCR3 mouse monoclonal antibody clone CEW33D (ThermoFisher Scientific, Waltham, MA); anti-human CXCR3 rabbit recombinant monoclonal antibody clone 6H1L8 (ThermoFisher Scientific, Waltham, MA); anti-human CXCR3 mouse monoclonal antibody clone #49801 (R&D Systems, Minneapolis, MN); anti-CXCR3 polyclonal antibody (cat #LS-B10183, LifeSpan Biosciences, Seattle, WA); and anti-CXCR3 polyclonal antibody (cat #NBP2-41250, Novus Biologicals, Littleton, CO). Other anti-CXCR3 antibodies are disclosed, e.g., in WO2013109974, WO2008094942, WO2001072334, and WO2005030793.

(vi) Immunosuppressant drugs. Immunosuppressant drugs may be used alone or in combination with the inhibitors of the present disclosure to treat an encephalopathy and/or to reduce inflammation. Immunosuppressant drugs are typically used to control a subject's immune system so that, for example, organs are not rejected in an organ, stem cell, or bone marrow transplant, or to control an autoimmune disease. Immunosuppressant drugs can help to reduce cell damage and inflammation. In particular embodiments, immunosuppressant drugs that can be used in the methods of the present disclosure include: corticosteroids (e.g., prednisone, budesonide, prednisolone, dexamethasone); Janus kinase inhibitors (e.g., tofacitinib); calcineurin inhibitors (e.g., cyclosporine, tacrolimus); mTOR inhibitors (sirolimus, everolimus); inosine monophosphate dehydrogenase (IMDH) inhibitors (e.g., azathioprine, leflunomide, mycophenolate); and biologics (e.g., abatacept, adalimumab, anakinra, certolizumab, etanercept, golimumab, infliximab, ixekizumab, natalizumab, rituximab, secukinumab, tocilizumab, ustekinumab, vedolizumab, basiliximab, and daclizumab).

In particular embodiments, compounds disclosed herein can be in the form of a pharmaceutically acceptable salt, a prodrug, a tautomer or a stereoisomer thereof.

(vii) Compositions. Inhibitors upstream of mTOR in the CSF1 pathway of neuroinflammation, inhibitors of chemokine receptor CXCR3, their functional derivatives, and/or immunosuppressant drugs (individually and collectively referred to as “active ingredients” herein) can be formulated for administration. Active ingredients can include neutral (non-salt) forms, as well as salt forms of the active ingredients. In particular embodiments, salts forms are pharmaceutically acceptable salts. Pharmaceutically acceptable salts are those which can be administered as drugs or pharmaceuticals to humans and/or animals and which, upon administration, retain at least some of the biological activity of the neutral or non-salt compound.

Salts of basic compounds can be prepared by methods known to those of skill in the art, for example, by treating the compound with an acid. Examples of inorganic acids include hydrochloric acid, hydrobromic acid, sulfuric acid, nitric acid, and phosphoric acid. Examples of organic acids include formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid.

Salts of acidic compounds can be prepared by methods known to those of skill in the art, for example, by treating the compound with a base. Examples of inorganic salts of acid compounds include alkali metal and alkaline earth salts, such as sodium salts, potassium salts, magnesium salts, and calcium salts; ammonium salts; and aluminum salts. Examples of organic salts of acid compounds include procaine, dibenzylamine, N-ethylpiperidine, N,N-dibenzylethylenediamine, and triethylamine salts.

Active ingredients can also include all stereoisomers of small molecule inhibitors and their functional derivatives upstream of mTOR in the CSF1 pathway of neuroinflammation, small molecule inhibitors of chemokine receptor CXCR3, and their functional derivatives, and small molecule immunosuppressant drugs including diastereomers and enantiomers, and mixtures of stereoisomers in any ratio, including racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

The active ingredients can be administered in prodrug form. Prodrugs are derivatives of the compounds, which are themselves relatively inactive but which convert into the active ingredients when introduced into the subject. Suitable prodrug formulations include peptide conjugates of the active ingredients and esters of active ingredients disclosed herein. Further discussion of suitable prodrugs is provided in H. Bundgaard, Design of Prodrugs, New York: Elsevier, 1985; in R. Silverman, The Organic Chemistry of Drug Design and Drug Action, Boston: Elsevier, 2004; in R. L. Juliano (ed.), Biological Approaches to the Controlled Delivery of Drugs (Annals of the New York Academy of Sciences, v. 507), New York: New York Academy of Sciences, 1987; and in E. B. Roche (ed.), Design of Biopharmaceutical Properties Through Prodrugs and Analogs (Symposium sponsored by Medicinal Chemistry Section, APhA Academy of Pharmaceutical Sciences, November 1976 national meeting, Orlando, Fla.).

In some embodiments, the compositions include active ingredients of at least 0.1% weight/volume (w/v) or weight/weight (w/w) of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Treatments described herein can be administered in combination with ingestion of a ketogenic diet. A ketogenic diet is a high fat, adequate protein, low carbohydrate diet. The key principle of the ketogenic diet is that a 3-5:1, more preferably 3.5-4.5:1, and most preferably a 4:1 ratio by weight of fat to non-fats (carbohydrate and protein) be maintained. Ideally, total daily calories should be divided into each meal and rationed, and the ratio of fat to non-fats should be kept the same in each meal. For additional information regarding ketogenic diets, see US20190091178A1, US20180213833A1, US20180110241A1, US20190191732A1, and US20180133194A1.

Particular embodiments of compositions can additionally include a secondary active ingredient. Exemplary secondary active ingredients include Coenzyme Q, including Coenzyme Q10; idebenone; MitoQ; acetylcarnitine (such as acetyl-L-carnitine or acetyl-DL-carnitine); palmitoylcarnitine (such as palmitoyl-L-carnitine or palmitoyl-DL-carnitine); carnitine (such as L-carnitine or DL-carnitine); quercetine; mangosteen; acai; uridine; N-acetyl cysteine (NAC); polyphenols, such as resveratrol; Vitamin A; Vitamin C; lutein; beta-carotene; lycopene; glutathione; fatty acids, including omega-3 fatty acids such as α-linolenic acid (ALA), eicosapentaenoic acid (EPA), and docosahexaenoic acid (DHA); lipoic acid and lipoic acid derivatives; Vitamin B complex; Vitamin B1 (thiamine); Vitamin B2 (riboflavin); Vitamin B3 (niacin, nicotinamide, or niacinamide); Vitamin B5 (pantothenic acid); Vitamin B6 (pyridoxine or pyridoxamine); Vitamin B7 (biotin); Vitamin B9 (folic acid, also known as Vitamin B11 or Vitamin M); Vitamin B12 (cobalamins, such as cyanocobalamin); inositol; 4-aminobenzoic acid; folinic acid; Vitamin E; other vitamins; and antioxidant compounds.

Exemplary pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, release modifiers, salts, solvents or co-solvents, stabilizers, surfactants, and delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and trimethylamine salts.

An exemplary chelating agent is EDTA.

Exemplary isotonic agents include polyhydric sugar alcohols including trihydric and higher sugar alcohols, such as glycerin, erythritol, arabitol, xylitol, sorbitol, and mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl and propyl paraben, catechol, resorcinol, cyclohexanol, and 3-pentanol.

Exemplary release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Delaware), poly(vinyl alcohol) (PVA), Brij® (Croda Americas, Wilmington, Delaware), sucrose acetate isobutyrate (SAIB), salts, and buffers.

Useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

The compositions disclosed herein can be formulated for administration by, for example, injection, inhalation, infusion, perfusion, lavage, ingestion, or absorption. The compositions disclosed herein can further be formulated for transdermal, intravenous, intradermal, intracranial, intracerebroventricular (ICV), intranasal, intraarterial, intranodal, intralymphatic, intraperitoneal, intralesional, intrathecal, intramuscular, intravesicular, oral and/or subcutaneous administration.

For injection, compositions can be formulated as aqueous solutions, such as in buffers including Hanks' solution, Ringer's solution, or physiological saline. The aqueous solutions can contain formulatory agents such as suspending, stabilizing, and/or dispersing agents. Alternatively, the formulation can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid formulations such as, for example, powders, capsules and tablets, suitable excipients include binders (gum tragacanth, acacia, cornstarch, gelatin) and fillers such as sugars, e.g. lactose, sucrose, mannitol and sorbitol. If desired, solid dosage forms can be sugar-coated or enteric-coated using standard techniques. Flavoring agents, such as peppermint, oil of wintergreen, cherry flavoring, orange flavoring, etc. can also be used.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly-soluble salts.

Additionally, compositions can be formulated as sustained-release systems utilizing semipermeable matrices of solid polymers containing at least one active ingredient. Various sustained-release materials have been established and are well known by those of ordinary skill in the art. Sustained-release systems may, depending on their chemical nature, release active ingredients following administration for a few weeks up to over 100 days.

Compositions may be formulated for administration locally via implantation of a membrane, sponge or another appropriate material onto which the active ingredient has been absorbed or encapsulated. Examples include chitosan sponges and collagen sponges.

In particular embodiments, compositions can be formulated with molecular linkages that facilitate targeted delivery to the central nervous system (e.g., brain and spinal cord). Particular embodiments targeting the central nervous system can utilize the transferrin receptor, using, for example, OX26, a peptidomimetic MAb that undergoes receptor mediated transcytosis following binding to the transferrin receptor. See, e.g., U.S. Pat. No. 6,372,250B1.

Compositions can also be formulated for intranasal delivery. When a nasal formulation is delivered deep and high enough into the nasal cavity, the olfactory mucosa is reached and drug transport into the central nervous system via the olfactory receptor neurons can occur, resulting in central nervous system delivery. Formulations and devices achieving such central nervous system delivery through nasal administration are described in, for example, US 2014/017048.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, compositions can be prepared to meet sterility, pyrogenicity, general safety, and purity standards as required by U.S. FDA Office of Biological Standards and/or other relevant foreign regulatory agencies.

(viii) Methods of Use. Methods disclosed herein include treating subjects (humans, veterinary animals, dogs, cats, reptiles, birds, etc.), livestock (horses, cattle, goats, pigs, chickens, etc.), and research animals (monkeys, rats, mice, fish, etc.) with compositions including an active ingredient and optionally a secondary active ingredient as disclosed herein. Treating subjects includes delivering therapeutically effective amounts. Therapeutically effective amounts include those that provide effective amounts, prophylactic treatments, and/or therapeutic treatments. In particular embodiments, the subject is a mouse. In particular embodiments, the subject is a human. In particular embodiments, the subject is a human under the age of 7 years old. In particular embodiments, the subject is a human under the age of 3 years old. In particular embodiments, the subject is a human under the age of 1 year old.

In particular embodiments, the methods disclosed herein include administering a composition to a subject, wherein the composition comprises a therapeutically effective amount of: an inhibitor upstream of mTOR in the CSF1 pathway of neuroinflammation, and an inhibitor of chemokine (C—X—C motif) receptor 3 (CXCR3). In particular embodiments, the methods disclosed herein include administering a composition to a subject, wherein the composition comprises a therapeutically effective amount of: an inhibitor upstream of mTOR in the CSF1 pathway of neuroinflammation, and an immunosuppressant drug. In particular embodiments, the methods disclosed herein include administering a composition to a subject, wherein the composition comprises a therapeutically effective amount of: an inhibitor of chemokine (C—X—C motif) receptor 3 (CXCR3), and an immunosuppressant drug. In particular embodiments, the methods disclosed herein include administering a composition to a subject, wherein the composition comprises a therapeutically effective amount of: an inhibitor upstream of mTOR in the CSF1 pathway of neuroinflammation, an inhibitor of chemokine (C—X—C motif) receptor 3 (CXCR3), and an immunosuppressant drug.

An “effective amount” is the amount of an active ingredient or composition necessary to result in a desired physiological change in a subject. Effective amounts are often administered for research purposes. In particular embodiments, effective amounts are observed using the Ndufs4 knockout (KO) mouse model of Leigh Syndrome. For example, Ndufs4KO mice can be assessed for: survival (medial survival is 55 days); clasping percentages; percent of ataxia; percent of circling; in the Rotarod test; in the automated CatWalk gait analysis tool; percent of cachexia; weight; glucose levels; lactate levels; respiratory function; amount of seizures; time to seizure; sensitivity to volatile anesthetics; area of lesions in the brain (e.g., brainstem and/or cerebellar peduncle); amount of microglia or astrocytes per area in a brain region (e.g., cortex and/or brainstem); levels of pro-inflammatory cytokines and/or chemokines including IFN-γ, IP-10 (CXCL10), and LIF; or a combination thereof.

Clasping involves an inward curling of the spine and a retraction of forelimbs or all limbs toward the midline of the body. Such clasping behavior is a widely used sign of neurological degeneration. Amount and or age of onset of clasping can be assessed visually.

In particular embodiments, effective amounts can be evidenced by reduced ataxia. Ataxia involves the loss of full control of bodily movements. Amount and or age of onset of ataxia can be assessed visually.

In particular embodiments, effective amounts can be evidenced by reduced circling. Circling involves repetitively tracing a loosely circular path. The circling can include changing body orientation, altering direction, and intermixing other behaviors. Amount and or age of onset of circling can be assessed visually.

The Rotarod test measures balance, coordination, and endurance. The test includes a circular rod turning at a constant or increasing speed. Animals placed on the rotating rod try to remain on it rather than fall onto a platform just below.

In particular embodiments, Rotarod parameters can include a beginning speed of 0 rpm with an acceleration rate of 0.1 rpm/s to a maximum speed of 40 rpm. Mice can undergo practice sessions on two consecutive days, with one session per day. The mice can be tested 24 hours after the second practice day, participating in three rotarod sessions on the testing day. Latency-to-fall times can be recorded.

In the CatWalk gait analysis tool, marked differences are noted between Ndufs4(−/−) and control mice in dynamic and static coordination. Variation of walking speed is significantly increased in Ndufs4(−/−) mice, suggesting hampered and uncoordinated gait. For additional information regarding the Ndufs4 knockout (KO) mouse model, see Johnson et al., Science, 2013, Dec. 20; 342(6165): 1524-1528.

In particular embodiments, effective amounts can be evidenced by reduced cachexia. Cachexia is a syndrome characterized by unintentional weight loss, progressive muscle wasting, and a loss of appetite. Cachexia onset described in the Examples is scored as the day of life when an individual animal begins showing weight loss after P37. In particular embodiments, cachexia can be assessed by an Animal cachexia score (ACASCO) that includes five components: (a) body and muscle weight loss, (b) inflammation and metabolic disturbances, (c) physical performance, (d) anorexia, and (e) quality of life measured using discomfort symptoms and behavioral tests (Betancourt et al. Animal Model Exp Med. 2019; 2(3):201-209). In particular embodiments, cachexia can be assessed in human subjects by the cachexia score (CASCO) that takes into consideration body weight loss and composition, inflammation/metabolic disturbances/immunosuppression, physical performance, anorexia, and quality of life (Argiles et al. J Cachexia Sarcopenia Muscle. 2011; 2(2):87-93). In particular embodiments, cachexia can be reduced in a subject administered a composition including a therapeutically effective amount of an inhibitor of the disclosure, as compared to cachexia in the subject not administered the composition, as measured by a relevant indicator such as a cachexia score. In particular embodiments, the onset of cachexia in a subject administered a composition including a therapeutically effective amount of an inhibitor of the disclosure can be delayed by at least 5 days, at least 10 days, at least 15 days, at least 20 days, at least 25 days, at least 30 days, at least 1 month, at least 2 months, at least 6 months, or longer, as compared to the onset of cachexia in the subject not administered the composition.

In particular embodiments, effective amounts can be evidenced by reduced hypoglycemia. Blood glucose levels can be measured with a point-of-care meter such as Prodigy Autocode® glucose meter (product #51850-3466188, Prodigy® Diabetes Care, LLC, Charlotte, NC). In mice blood can be obtained by a tail-prick method (e.g., described in Stokes et al. Elife 2021; 10:e65400). In humans blood can be obtained from a finger prick. In particular embodiments, glucose level in a subject administered a composition including a therapeutically effective amount of an inhibitor of the disclosure is decreased less than 10%, less than 9%, less than 8%, less than 7%, less than 6%, less than 5%, less than 4%, less than 3%, less than 2%, less than 1%, or less, as compared to the glucose level in the subject not administered the composition at a given time point.

In particular embodiments, effective amounts can be evidenced by reduced hyperlactemia. Blood lactate levels can be measured with a point-of-care meter such as a lactate assay meter, product #40828, from Nova Biomedical (Waltham, MA). In mice blood can be obtained by a tail-prick method (e.g., described in Stokes et al. Elife 2021; 10:e65400). In humans blood can be obtained from a vein or artery, or a sample of cerebrospinal fluid can be obtained by a spinal tap to measure a lactate level. In particular embodiments, lactate levels in a subject can be measured in response to a glucose bolus in a glucose tolerance test (GTT). In particular embodiments, lactate levels in a subject can be measured in response to exposure to a volatile anesthetic. In particular embodiments, lactate levels in a subject administered a composition including a therapeutically effective amount of an inhibitor of the disclosure is increased less than 10%, less than 9%, less than 8%, less than 7%, less than 6%, less than 5%, less than 4%, less than 3%, less than 2%, less than 1%, or less, as compared to the lactate level of the subject not administered the composition at a given time point.

Respiratory function can be measured using whole-body plethysmography. In particular embodiments, paired 300 ml recording and reference chambers are continuously ventilated (e.g., 150 ml/min) with either normal air (e.g., 79% nitrogen and 21% oxygen) or a hypercapnic gas mixture (e.g., 74% nitrogen, 21% oxygen, and 5% CO₂). Pressure differences between the recording and reference chambers can be measured and digitized to visualize respiratory pattern, and simultaneous video recordings can be performed to differentiate resting breathing activity from exploratory sniffing and grooming behaviors. Untreated and treated (e.g., pexidartinib 300 mg/kg/day) control mice and mice modeling an encephalopathy can be allowed to acclimate to the chambers prior to acquisition of respiratory activity in normal air. The respiratory response to hypercapnia (excessive CO₂ in the bloodstream) can then be tested by ventilating the chambers with hypercapnic gas. Respiratory frequency, and breath-to-breath irregularity scores (irreg.score=ABS((N-(N−1))/N)) of frequency and amplitude (peak inspiratory airflow) can be quantified during periods of resting breathing.

Seizure activity in mice can be assessed by observing whether symptoms on the Racine behavioral scale or Pinel and Rovner scale are present. In particular embodiments, symptoms include: abnormal oroalimentary movements (dropping of the jaw repeatedly, atypical gnawing or chewing movements); repeat head nodding; anterior limb clonus (twitching/jumping while making no contact with the face); dorsal extension/rearing; loss of balance and falling; and/or violently running/jumping (‘popcorning’).

In particular embodiments, effective amounts can be evidenced by reduced sensitivity to volatile anesthetics. Sensitivity to volatile anesthetics can be determined by measuring the minimum alveolar concentration (MAC). MAC provides a correlation between the dose of an anesthetic and immobility. (Lobo et al. (2020) StatPearls, World Wide Web at ncbi.nlm.nih.gov/books/NBK532974/). In particular embodiments, MAC refers to the concentration of inhaled anesthetic within the alveoli at which 50% of subjects do not move in response to a surgical stimulus. MAC can be expressed as volumes percent of alveolar (end-tidal) gas at one atmosphere pressure at sea level (i.e., 760 mm Hg). In particular embodiments, volatile anesthetics can include nitrous oxide, isoflurane, desflurane, halothane, and sevoflurane. Mice can be exposed to an anesthetic by using a vaporizer at a particular flow rate (e.g., 3-4 liters/min) through a humidifier in-line. One hundred percent oxygen can be used as the carrier gas. In particular embodiments, a plexiglass exposure chamber and humidifier can be pre-warmed to 38° C. and maintained at this temperature throughout the exposure using a circulating water heating pad. In particular embodiments, sensitivity to volatile anesthetics can be reduced in a subject administered a composition including a therapeutically effective amount of an inhibitor of the disclosure, as compared to sensitivity to volatile anesthetics in the subject not administered the composition, as measured by a relevant test such as MAC.

Area of lesions in the brainstem and/or cerebellar peduncle can be assessed by methods known in the art, for example, immunofluorescence and immunocytochemistry assays to assess presence of particular biomarkers of lesions such as ionized calcium binding adaptor molecule 1 (Iba1) expressed by activated microglia and glial fibrillary acidic protein (GFAP) expressed by activated astrocytes.

In particular embodiments, effective amounts can be evidenced by reduced microglial and/or astrocyte activation. Microglial and/or astrocyte activation can be assessed using positron emission tomograph (PET) imaging as well as peripheral biomarker analysis. Other assays that can be used to assess microglial and/or astrocyte activation include enzyme-linked immunosorbent assays (ELISA), enzyme-linked immunosorbent spot assays (ELISPOT), western blots assays, in-cell western assays, fluorescence-activated cell sorting assays (FACS), immunocytochemistry assays, colorimetric assays, fluorescence and luminescence assays, quantitative reverse transcription-polymerase chain reaction assays (Quantitative RT-PCR), high-performance liquid chromatography assays (HPLC), electrophysiological measurement assays, and mass spectrometry assays (see, e.g., Horvath, et al., J Neurochem. 2008; 107:557-569; Mirzoeva, et al., J Med Chem. 2002; 45:563-566; Perego et al., J Neuroinflammation. 2011; 8:174. Chhor, et al., Brain Behav Immun. 2013; 32:70-85; Hetrick, et al., Annu Rev Anal Chem (Palo Alto Calif) 2009; 2:409-433; Barger et al., J Neurochem. 2001; 76:846-854; Zhou, et al., Anal Biochem. 1997; 253:162-168; Mohanty, et al., J Immunol Methods. 1997; 202:133-141; Tarpey, et al., Am J Physiol Regul Integr Comp Physiol. 2004; 286:R431-R444; Coelho-Santos, et al. J Neuroinflammation. 2012; 9:103; Wang Za, et al., Pure Appl Chem. 2001; 73:1599-1611; Zhao, et al., J Neurochem. 2004; 88:169-180; Schilling, et al., J Physiol. 2004; 557:105-120; Farber, et al., Pflugers Arch. 2006; 452:615-621). In particular embodiments, glial cell activation includes induction of expression and release of cytokines and chemokines from glial cells; proliferation of glial cells; alterations in morphology of glial cells; redistribution of cell-surface markers of glial cells (e.g., increased surface expression of Iba1, GFAP, CD68); migration of glial cells towards sites of injury or infection; increase in phagocytic efficiency of microglia; or a combination thereof. In particular embodiments, microglial activation can be assessed by measuring the presence or level of Iba1 In particular embodiments, astrocyte activation can be assessed by measuring the presence or level of GFAP.

In particular embodiments, effective amounts can be evidenced by reduced leukocyte proliferation. Leukocytes are white blood cells that function to combat infection and other diseases as part of a subject's immune system. In particular embodiments, leukocytes include lymphocytes (T cells and B cells), granulocytes (neutrophils, eosinophils, and basophils), monocytes, macrophages, and microglia. In particular embodiments, leukocytes include microglia. Leukocyte proliferation can be measured by techniques known in the art, including use of: [³H]thymidine incorporation; bromo-2′-deoxyuridine (BrdU) incorporation; colorimetric MTT (tetrazolium dye) assay; a cell division tracking dye (e.g., carboxyfluorescein diacetate succinimidyl ester (CFSE), cell trace violet (CTV), or cell proliferation dye efluor 670 (CPD)) (Hawkins et al. Nature Protocols 2007; 2:2057-2067; Quah and Parish. Journal of Immunological Methods 2012; 379(1-2):1-14); light absorbance on an ELISA reader in the yellow wave length (Gao et al. Journal of Immunoassay 1998; 19 (2-3):129-143); a micro whole blood technique (Sheth et al. Annals of Saudi Medicine 1993; density gradient centrifugation (Boyum A Scand J Clin Lab Invest 21. 1968; (suppl) 97:7); flow cytometry detection of cells labeled with fluorescent antibodies to particular leukocyte markers (e.g., CD3, CD4, CD11a, CD11b, CD15, CD16, CD24, CD45, CD91, CD114, CD182); and electrochemical methods such as cyclic voltammetry (Nikbakht et al. Scientific Reports 2019; 9:4503). In particular embodiments, the level or amount of leukocytes can be reduced in a subject administered a composition including a therapeutically effective amount of an inhibitor of the disclosure by at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more, as compared to the level or amount of leukocytes in the subject not administered the composition.

Levels of pro-inflammatory cytokines and/or chemokines including IFN-γ, IP-10 (CXCL10), and LIF can be measured by techniques including RT-PCR, real time quantitative PCR, gene array analysis, northern blot analysis, ribonuclease protection assay, flow cytometry, ELISPOT, western blot analysis, and ELISA. Assays to detect multiple cytokines and/or chemokines simultaneously can be in a high-throughput format, such as flow cytometric multiplex arrays (e.g., Millipore MCYTMAG-70K-PX32 Milliplex MAP Mouse Cytokine/Chemokine Magnetic Bead Panel Multiplex panel; cytometric bead array (CBA) system (BD Biosciences, Franklin Lakes, NJ); and Luminex multi-analyte profiling (xMAP) technology (Luminex Corporation, Austin, TX)), multiplex ELISA (e.g., from Quansys Biosciences, Logan, UT), and electrochemiluminescence technology with multiple specific capture antibodies coated at corresponding spots on an electric wired microplate (Meso Scale Discovery, Rockville, MD).

A “prophylactic treatment” includes a treatment administered to a subject who does not display signs or symptoms of an encephalopathy or only displays early signs or symptoms of the encephalopathy such that treatment is administered for the purpose of delaying, reducing, preventing, or decreasing the risk of developing the encephalopathy further. Thus, a prophylactic treatment functions as a preventative treatment against an encephalopathy.

A “therapeutic treatment” includes a treatment administered to a subject who displays symptoms or signs of an encephalopathy and is administered to the subject for the purpose of resolving or reducing the effects of the encephalopathy.

The actual dose amount administered to a particular subject can be determined by a physician, veterinarian, or researcher taking into account parameters such as physical and physiological factors including target area; body weight; type and severity of encephalopathy or resulting condition; prospective conditions; previous or concurrent therapeutic interventions; idiopathy of the subject; and route of administration.

A composition including a therapeutically effective amount of an active ingredient(s) and optionally a secondary active ingredient disclosed herein can be administered to a subject in a clinically safe and effective manner, including one or more separate administrations of the composition.

Useful doses can often range from 0.1 to 5 μg/kg. In other examples, a dose can include 1 μg/kg, 10 μg/kg, 50 μg/kg, 75 μg/kg, 100 μg/kg, 150 μg/kg, 200 μg/kg, 500 μg/kg, 1000 μg/kg, 0.1 to 5 mg/kg, or from 0.5 to 1 mg/kg. In other examples, a dose can include 1 mg/kg, 10 mg/kg, 25 mg/kg, 50 mg/kg, 55 mg/kg, 100 mg/kg, 250 mg/kg, 500 mg/kg, 750 mg/kg, 1000 mg/kg, or more. In particular embodiments, a useful dose is 200 mg/kg/day.

Each of the described doses of active ingredients can be an active ingredient alone, or in combination with one or more other active ingredients. In particular embodiments, when included in combinations to produce a dose, such as a dose stated herein, the substituents in the combination can be provided in exemplary ratios such as: 1:1; 1:1.25; 1:1.5; 1:1.75; 1:8; 1:1.2; 1:1.25; 1:1.3; 1:1.35; 1:1.4; 1:1.5; 1:1.75; 1:2; 1:3; 1:4; 1:5; 1:6; 1:7; 1:8; 1:9; 1:10; 1:15; 1:20; 1:30; 1:40; 1:50; 1:60; 1:70; 1:80; 1:90; 1:100; 1:200; 1:300; 1:400; 1:500; 1:600; 1:700; 1:800; 1:900; 1:1000; 1:1:1; 1:2:1; 1:3:1; 1:4:1; 1:5:1; 1:10:1; 1:2:2; 1:2:3; 1:3:4; 1:4:2; 1:5:3; 1:10:20; 1:2:1:2; 1:4:1:3; 1:100:1:1000; 1:25:30:10; 1:4:16:3; 1:1000:5:15; 1:2:3:10; 1:5:15:45; 1:50:90:135; 1:1.5:1.8:2.3; 1:10:100:1000 or additional beneficial ratios depending on the number and identity of substituents in a combination to reach the stated dosage. The substituents in a combination can be provided within the same composition or within different compositions.

Therapeutically effective amounts can be achieved by administering single or multiple doses during the course of a treatment regimen (e.g., QID, TID, BID, daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months, or yearly).

Effective prophylactic and/or therapeutic treatments against encephalopathies can be demonstrated using a variety of different assessments depending on the type of encephalopathy, severity of encephalopathy, and type and age of subject. For example, there are several known assessment programs for pediatricians to evaluate children. For physical abilities, the Pediatric Evaluation of Disability Inventory (PEDI) can be used (see Haley, S. M., Coster, W. J., Ludlow, L. H., Haltiwanger, J. T., & Andrellos, P. J. (1992). Pediatric Evaluation of Disability Inventory: Development, Standardization, and Administration Manual, Version 1.0. Boston, Mass.: Trustees of Boston University, Health and Disability Research Institute). PEDI enables evaluation of functional disabilities using standardized score forms. The PEDI can be used to assess key functional capabilities and performance in children ages six months to seven years, and to evaluate older children whose functional abilities are lower than those of seven-year-olds without disabilities. PEDI can be used to identify functional deficits and monitor treatment progress.

For neuro-psychiatric evaluation, the NEPSY-II assessment (Korkman, Marit; Kirk, Ursula; & Kemp, Sally. (2007) NEPSY-II-Second Edition, San Antonio, Tex.: Pearson) can be used to gauge neuropsychological development. Testing in children 3-4 years of age can assess six functional domains: attention and executive functions; language and communication; sensorimotor functions; visuospatial functions; learning and memory; and social perception.

A scale to monitor progression and treatment of mitochondrial diseases in children, commonly known as the Newcastle Paediatric Mitochondrial Disease Scale (NPMDS), monitors the biophysical markers of disease progression. The scale is based around four domains: current function; system-specific involvement; current clinical assessment; and quality of life, as described by Phoenix et al., in “A scale to monitor progression and treatment of mitochondrial disease in children,” Neuromuscular Disorders (2006) 16 814-820.

Additionally Wolf et al., “Mitochondrial disorders: a proposal for consensus diagnostic criteria in infants and children,” Neurology (2002) 59 (9) 1402-1405 describes diagnostic criteria in infants and children with mitochondrial diseases.

Standard motor function tests can be used to assess many symptoms, including tests used by physical therapists, occupational therapists, and rehabilitation medicine specialists to assess patient function. For example, to assess the level or degree of ataxia in a subject, tests including a tapping test for the arm, a tapping test for the legs, a quantified finger-to-nose test, and a modified Romberg test can be used. In particular embodiments, a clinical Scale for the Assessment and Rating of Ataxia (SARA) can be used that includes eight measurements related to gait, stance, sitting, speech disturbance, finger-chase test, nose-finger test, fast alternating movements and heel-shin test (Schmitz-Hübsch et al. Neurology. 2006; 66:1717-1720; Weyer et al. Movement Disorders 2007; 22:1633-1637). In particular embodiments, an International Cooperative Ataxia Rating Scale (ICARS) test can be used (Trouillas et al. J Neurol Sci. 1997; 145:205-211). As another example, neurological assessments can be performed using tests such as the Glasgow Coma Scale (GCS), which measures eye opening, verbal response, and motor response (Majdan et al. Journal of Neurotrauma 2015; 32(2):101-108). As a further example, the conscious state of a subject can be measured by the AVPU (Alert, Verbal, Pain, and Unresponsive) scale (Romanelli, D & Farrell, M W 2021, ‘AVPU Score’, StatPearls, on World Wide Web at ncbi.nlm.nih.gov/books/NBK538431/).

Therapeutically effective amounts can be evidenced by reduced tremors, reduced spasms (including myoclonic spasms), reduced frequency of seizures, reduced hypotonia, reduced weakness, reduced fatigue, reduced ataxia, and/or reduced difficulty in walking. Therapeutically effective amounts can also be evidenced by reduced gastrointestinal abnormalities, reduced eye abnormalities (including vision loss), reduced nystagmus, reduced optic atrophy, reduced hearing loss, reduced abnormal or absent reflexes, reduced difficulty in breathing, improved respiratory function, reduced difficulty in speaking, reduced difficulty in swallowing, reduced failure to thrive, reduced low body weight, reduced cachexia, reduced growth retardation, reduced impaired kidney function, reduced terminal stupor, reduced hypoglycemia, reduced lactic acidosis, and reduced sensitivity to volatile anesthetics. In infants, therapeutically effective amounts can also be evidenced by improved sucking ability, improved head control, improved motor skills, improved appetite, reduced vomiting, reduced irritability, reduced crying, and/or reduced seizures. For additional therapeutic criteria, see Finsterer, J., “Leigh and Leigh-like syndrome in children and adults,” Pediatr. Neurol. 2008; 39:223-235).

In additional embodiments, treatment according to the disclosure can produce in a patient an adequate reduction or alleviation of one or more of the observable characteristics of an encephalopathy by an amount that is discernible to a human observer, such as a parent, physician or caretaker, without the use of special devices such as imaging technology, microscopes or chemical analytical devices. For example, treatment according to the disclosure can produce an observable reduction of ataxia and difficulty in walking, wherein a patient that was bed-bound and lethargic prior to treatment is able, after treatment, to walk with assistance; balance, including balancing on one foot; ride a tricycle; walk up steps; sit without assistance; independently stand and support himself or herself by holding on to a table or a fixed object for at least one minute; turn and scoot or slide while sitting; move his or her extremities purposefully, as in giving a “high-five” gesture; and perform fine motor tasks such as grasping small objects. Treatment according to the disclosure can produce an observable reduction of speech problems, such as speaking in complete sentences, improved enunciation, counting aloud, having increased voice and word association; and can improve cognitive skills, such as asking “why,” and responding to verbal communication appropriately. Treatment according to the disclosure can produce observable improved sleep patterns, normalization of gastrointestinal problems, improved hand-eye coordination, and improved breathing.

In particular embodiments, effective amounts can be evidenced by reduced neurological symptoms such as ataxia, dysarthria, hypotonia, clumsiness, tremors, and/or muscle spasms. In particular embodiments, the incidence or amount of neurological symptoms can be reduced in a subject administered a composition including a therapeutically effective amount of an inhibitor of the disclosure by at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more, as compared to incidence or amount of neurological symptoms in the subject not administered the composition, as measured by a relevant test. In particular embodiments, the onset of neurological symptoms can be delayed in a subject administered a composition including a therapeutically effective amount of an inhibitor of the disclosure by at least 5 days, at least 10 days, at least 15 days, at least 20 days, at least 25 days, at least 30 days, at least 1 month, at least 2 months, at least 6 months, or longer, as compared to onset of neurological symptoms in the subject not administered the composition, as measured by a relevant test.

In particular embodiments, effective amounts can be evidenced by improved respiratory function. Respiratory function can be assessed by body (also called lung) plethysmography, a pulmonary function test. A body plethysmography can measure how much air is in the lungs after a subject takes in a deep breath (total lung capacity, TLC); the amount of air left in the lungs after a subject exhales normally (functional residual capacity, FRC); and/or the amount of air left in the lungs after a subject exhales as much as possible (residual capacity, RC). In particular embodiments, a subject undergoing a body plethysmography is seated in a transparent chamber wearing a nose clip to shut off air to the nostrils and breathing through a mouthpiece. Changes in pressure and amount of air in the chamber and changes in pressure against the mouthpiece can be used to measure TLC, FRC, and RC. The test is based upon Boyle's Law, a scientific principle that describes the ability to measure the volume of a gas and determine its pressure, or vice versa, given a constant temperature. In particular embodiments, respiratory function can be improved in a subject administered a composition including a therapeutically effective amount of an inhibitor of the disclosure, as compared to the subject not administered the composition, as measured by a relevant test such as plethysmography.

In particular embodiments, effective amounts can be evidenced by reduced frequency of seizures. Seizures can be measured in a number of ways including: diary entries, shake detectors, electrodermal response, video-eletroencephalogram, invasive eletroencephalogram, and imaging tests (e.g., magnetic resonance imaging (MRI), computerized tomography (CT), positron emission tomography (PET), and single-photon emission computerized tomography (SPECT). In particular embodiments, frequency of seizures can be reduced in a subject administered a composition including a therapeutically effective amount of an inhibitor of the disclosure, as compared to the subject not administered the composition, as measured by a relevant test.

(ix) Kits. The current disclosure also includes kits including inhibitors upstream of mTOR in the CSF1 pathway of neuroinflammation, inhibitors of CXCR3, functional derivatives thereof, and/or immunosuppressant drugs to treat genetic or environmental encephalopathies, to reduce glial cell activation, to reduce leukocyte proliferation, and/or to reduce neuroinflammation. In particular embodiments, inhibitors upstream of mTOR in the CSF1 pathway of neuroinflammation include: pexidartinib, and/or functional derivatives thereof; and PLX 5622, and/or functional derivatives thereof. In particular embodiments, inhibitors of CXCR3 include: AMG487; TAK-779; SCH 546738; NBI-74330; PS372424; and/or functional derivatives thereof. The kits can further include an immunosuppressant drug. In particular embodiments, immunosuppressant drugs include: corticosteroids; Janus kinase inhibitors; calcineurin inhibitors; mTOR inhibitors; inosine monophosphate dehydrogenase (IMDH) inhibitors; and biologics. The kits can further include a secondary active ingredient as described herein. In particular embodiments, a secondary active ingredient includes: Coenzyme Q; idebenone; MitoQ; acetylcarnitine; palmitoylcarnitine; carnitine; quercetine; mangosteen; acai; uridine; N-acetyl cysteine (NAC); polyphenols; Vitamin A; Vitamin C; lutein; beta-carotene; lycopene; glutathione; fatty acids; lipoic acid and derivatives thereof; Vitamin B complex; Vitamin B1; Vitamin B2; Vitamin B3; Vitamin B5; Vitamin B6; Vitamin B7; Vitamin B9; Vitamin B12; inositol; 4-aminobenzoic acid; folinic acid; Vitamin E; other vitamins; and antioxidant compounds. Kits include a container and a label. The label indicates that the inhibitor upstream of mTOR in the CSF1 pathway of neuroinflammation, inhibitors of CXCR3, functional derivatives thereof, and/or immunosuppressant drugs and optionally secondary active ingredients are useful to treat genetic or environmental encephalopathies, to reduce glial cell activation, to reduce leukocyte proliferation, to reduce neurologic symptoms, to improve respiratory function, to reduce frequency of seizures, to reduce cachexia, to reduce hypoglycemia, to reduce hyperlactemia, to reduce sensitivity to volatile anesthetics, and/or to reduce neuroinflammation. The label may also provide instructions for use, in particular examples, including directions for treatment.

### EXEMPLARY EMBODIMENTS

1. A method of treating an encephalopathy in a subject in need thereof including administering a composition to the subject, wherein the composition includes a therapeutically effective amount of
an inhibitor upstream of mTOR in the CSF1 pathway of neuroinflammation;
an inhibitor of chemokine (C—X—C motif) receptor 3 (CXCR3); and/or
an immunosuppressant drug, thereby treating the encephalopathy.
   2. The method of embodiment 1, wherein the encephalopathy includes a genetic encephalopathy or an environmental encephalopathy.
   3. The method of embodiment 1 or 2, wherein the encephalopathy includes a mitochondrial encephalopathy.
   4. The method of any of embodiments 1-3, wherein the encephalopathy includes Leigh Syndrome or Wernicke encephalopathy.
   5. A method of reducing glial cell activation in a subject in need thereof including administering a composition to the subject, wherein the composition includes a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug, thereby reducing the glial cell activation, as compared to glial cell activation when the subject is not administered the composition.
   6. A method of reducing neuroinflammation in a subject in need thereof including administering a composition to the subject, wherein the composition includes a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug,
   thereby reducing the neuroinflammation, as compared to neuroinflammation in the subject when the subject is not administered the composition.
   7. A method of reducing leukocyte proliferation in a subject in need thereof including administering a composition to the subject, wherein the composition includes a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug,
   thereby reducing the leukocyte proliferation in the subject, as compared to leukocyte proliferation in the subject when the subject is not administered the composition.
   8. A method of improving respiratory function in a subject in need thereof including administering a composition to the subject, wherein the composition includes a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug,
   thereby improving the respiratory function in the subject, as compared to respiratory function in the subject when the subject is not administered the composition.
   9. A method of reducing frequency of seizures in a subject in need thereof including administering a composition to the subject, wherein the composition includes a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug,
   thereby reducing the frequency of seizures in the subject, as compared to frequency of seizures in the subject when the subject is not administered the composition.
   10. A method of reducing hypoglycemia in a subject in need thereof including administering a composition to the subject, wherein the composition includes a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug,
   thereby reducing the hypoglycemia in the subject, as compared to hypoglycemia in the subject when the subject is not administered the composition.
   11. A method of reducing hyperlactemia in a subject in need thereof including administering a composition to the subject, wherein the composition includes a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug,
   thereby reducing the hyperlactemia in the subject, as compared to hyperlactemia in the subject when the subject is not administered the composition.
   12. A method of reducing sensitivity to a volatile anesthetic in a subject including administering a composition to the subject, wherein the composition includes a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug,
   thereby reducing the sensitivity to the volatile anesthetic in the subject, as compared to sensitivity to the volatile anesthetic in the subject not administered the composition.
   13. The method of embodiment 12, wherein the volatile anesthetic includes nitrous oxide, isoflurane, desflurane, halothane, and sevoflurane.
   14. The method of any of embodiments 1-13, wherein the inhibitor upstream of mTOR in the CSF1 pathway of neuroinflammation binds the CSF1 receptor and interferes with binding by the natural CSF-1 ligand.
   15. The method of any of embodiments 1-14, wherein the inhibitor upstream of mTOR in the CSF1 pathway of neuroinflammation includes PLX 5622 and/or a functional derivative thereof.
   16. The method of any of embodiments 1-15, wherein the inhibitor upstream of mTOR in the CSF1 pathway of neuroinflammation includes Pexidartinib and/or a functional derivative thereof.
   17. The method of any of embodiments 1-16, wherein the inhibitor upstream of mTOR in the CSF1 pathway of neuroinflammation inhibits the P110γ microglia specific catalytic subunit of PI3K.
   18. The method of any of embodiments 1-17, wherein the inhibitor upstream of mTOR in the CSF1 pathway of neuroinflammation includes IPI-549 and/or a functional derivative thereof.
   19. The method of any of embodiments 1-18, wherein the inhibitor of CXCR3 binds CXCR3 and interferes with binding by a natural ligand of CXCR3.
   20. The method of embodiment 19, wherein the natural ligand of CXCR3 is interferon γ-inducible 10 kD Protein (IP-10).
   21. The method of any of embodiments 1-20, wherein the inhibitor of CXCR3 includes: AMG487; TAK-779; SCH 546738; NBI-74330; PS372424; and/or functional derivatives thereof.
   22. The method of any of embodiments 1-21, wherein the immunosuppressant drug includes a corticosteroid, a Janus kinase inhibitor, a calcineurin inhibitor, an mTOR inhibitor, an inosine monophosphate dehydrogenase (IMDH) inhibitor, a biologic, or a combination thereof.
   23. The method of embodiment 22, wherein the corticosteroid includes prednisone, budesonide, prednisolone, dexamethasone, or a combination thereof.
   24. The method of embodiment 22 or 23, wherein the Janus kinase inhibitor includes tofacitinib.
   25. The method of any of embodiments 22-24, wherein the calcineurin inhibitor includes cyclosporine, tacrolimus, or a combination thereof.
   26. The method of any of embodiments 22-25, wherein the mTOR inhibitor includes sirolimus, everolimus, or a combination thereof.
   27. The method of any of embodiments 22-26, wherein the IMDH inhibitor includes azathioprine, leflunomide, mycophenolate, or a combination thereof.
   28. The method of any of embodiments 22-27, wherein the biologic includes abatacept, adalimumab, anakinra, certolizumab, etanercept, golimumab, infliximab, ixekizumab, natalizumab, rituximab, secukinumab, tocilizumab, ustekinumab, and vedolizumab, basiliximab, daclizumab, or a combination thereof.
   29. The method of any of embodiments 1-28, further including recommending that the subject ingest a ketogenic diet.
   30. The method of any of embodiments 1-29, further including administering to the subject a composition including a secondary active ingredient.
   31. The method of embodiment 30, wherein the secondary active ingredient is within a ketogenic diet.
   32. The method of embodiment 30 or 31, wherein the secondary active ingredient includes Coenzyme Q, idebenone, acetylcarnitine, palmitoylcarnitine, carnitine, quercetine, mangosteen, acai, uridine, N-acetyl cysteine, a polyphenol, Vitamin A, Vitamin C, lutein, beta-carotene, lycopene, glutathione, a fatty acid, lipoic acid, a Vitamin B complex, Vitamin B1, Vitamin B2, Vitamin B3, Vitamin B5, Vitamin B6, Vitamin B7, Vitamin B9, Vitamin B12, inositol, 4-aminobenzoic acid, folinic acid, and/or Vitamin E.
   33. The method of any of embodiments 1-32, wherein the subject is a mouse.
   34. The method of any of embodiments 1-32, wherein the subject is a human.
   35. The method of any of embodiments 1-32 and 34, wherein the subject is a human under the age of 7 years old.
   36. The method of any of embodiments 1-32, 34 and 35, wherein the subject is a human under the age of 3 years old.
   37. The method of any of embodiments 1-32, and 34-36, wherein the subject is a human under the age of 1 year old.
   38. A pharmaceutical composition including:
an inhibitor upstream of mTOR in the CSF1 pathway of neuroinflammation;
an inhibitor of chemokine (C—X—C motif) receptor 3 (CXCR3);
an immunosuppressant drug; and/or
a secondary active ingredient.
   39. The pharmaceutical composition of embodiment 38, wherein the inhibitor upstream of mTOR in the CSF1 pathway of neuroinflammation includes PLX 5622, Pexidartinib, or IPI-549, or a combination thereof.
   40. The pharmaceutical composition of embodiment 38 or 39, wherein the inhibitor upstream of mTOR in the CSF1 pathway of neuroinflammation includes a functional derivative of PLX 5622, Pexidartinib, or IPI-549, or a combination thereof.
   41. The pharmaceutical composition of any of embodiments 38-40, wherein the inhibitor of CXCR3 includes: AMG487; TAK-779; SCH 546738; NBI-74330; PS372424; and/or functional derivatives thereof.
   42. The pharmaceutical composition of any of embodiments 38-41, wherein the immunosuppressant drug includes a corticosteroid, a Janus kinase inhibitor, a calcineurin inhibitor, an mTOR inhibitor, an inosine monophosphate dehydrogenase (IMDH) inhibitor, a biologic, or a combination thereof.
   43. The pharmaceutical composition of embodiment 42, wherein the corticosteroid includes prednisone, budesonide, prednisolone, dexamethasone, or a combination thereof.
   44. The pharmaceutical composition of embodiment 42 or 43, wherein the Janus kinase inhibitor includes tofacitinib.
   45. The pharmaceutical composition of any of embodiments 42-44, wherein the calcineurin inhibitor includes cyclosporine, tacrolimus, or a combination thereof.
   46. The pharmaceutical composition of any of embodiments 42-45, wherein the mTOR inhibitor includes sirolimus, everolimus, or a combination thereof.
   47. The pharmaceutical composition of any of embodiments 42-46, wherein the IMDH inhibitor includes azathioprine, leflunomide, mycophenolate, or a combination thereof.
   48. The pharmaceutical composition of any of embodiments 42-47, wherein the biologic includes abatacept, adalimumab, anakinra, certolizumab, etanercept, golimumab, infliximab, ixekizumab, natalizumab, rituximab, secukinumab, tocilizumab, ustekinumab, and vedolizumab, basiliximab, daclizumab, or a combination thereof.
   49. The pharmaceutical composition of any of embodiments 42-48, wherein the secondary active ingredient is within a ketogenic diet.
   50. The pharmaceutical composition of any of embodiments 42-49, wherein the secondary active ingredient includes Coenzyme Q, idebenone, acetylcarnitine, palmitoylcarnitine, carnitine, quercetine, mangosteen, acai, uridine, N-acetyl cysteine, a polyphenol, Vitamin A, Vitamin C, lutein, beta-carotene, lycopene, glutathione, a fatty acid, lipoic acid, a Vitamin B complex, Vitamin B1, Vitamin B2, Vitamin B3, Vitamin B5, Vitamin B6, Vitamin B7, Vitamin B9, Vitamin B12, inositol, 4-aminobenzoic acid, folinic acid, and/or Vitamin E.
   51. A kit including:
an inhibitor upstream of mTOR in the CSF1 pathway of neuroinflammation;
an inhibitor of chemokine (C—X—C motif) receptor 3 (CXCR3);
an immunosuppressant drug; and/or
a secondary active ingredient.
   52. The kit of embodiment 51, wherein the inhibitor upstream of mTOR in the CSF1 pathway of neuroinflammation includes PLX 5622, Pexidartinib, IPI-549, or a combination thereof.
   53. The kit of embodiment 51 or 52, wherein the inhibitor upstream of mTOR in the CSF1 pathway of neuroinflammation includes a functional derivative of PLX 5622, Pexidartinib, or IPI-549, or a combination thereof.
   54. The kit of any of embodiments 51-53, wherein the inhibitor of CXCR3 includes: AMG487; TAK-779; SCH 546738; NBI-74330; PS372424; and/or functional derivatives thereof.
   55. The kit of any of embodiments 51-54, wherein the immunosuppressant drug includes a corticosteroid, a Janus kinase inhibitor, a calcineurin inhibitor, an mTOR inhibitor, an inosine monophosphate dehydrogenase (IMDH) inhibitor, a biologic, or a combination thereof.
   56. The kit of embodiment 55, wherein the corticosteroid includes prednisone, budesonide, prednisolone, dexamethasone, or a combination thereof.
   57. The kit of embodiment 55 or 56, wherein the Janus kinase inhibitor includes tofacitinib.
   58. The kit of any of embodiments 55-57, wherein the calcineurin inhibitor includes cyclosporine, tacrolimus, or a combination thereof.
   59. The kit of any of embodiments 55-58, wherein the mTOR inhibitor includes sirolimus, everolimus, or a combination thereof.
   60. The kit of any of embodiments 55-59, wherein the IMDH inhibitor includes azathioprine, leflunomide, mycophenolate, or a combination thereof.
   61. The kit of any of embodiments 55-60, wherein the biologic includes abatacept, adalimumab, anakinra, certolizumab, etanercept, golimumab, infliximab, ixekizumab, natalizumab, rituximab, secukinumab, tocilizumab, ustekinumab, and vedolizumab, basiliximab, daclizumab, or a combination thereof.
   62. The kit of any of embodiments 55-61, wherein the secondary active ingredient is within a ketogenic diet.
   63. The kit of any of embodiments 55-62, wherein the secondary active ingredient includes Coenzyme Q, idebenone, acetylcarnitine, palmitoylcarnitine, carnitine, quercetine, mangosteen, acai, uridine, N-acetyl cysteine, a polyphenol, Vitamin A, Vitamin C, lutein, beta-carotene, lycopene, glutathione, a fatty acid, lipoic acid, a Vitamin B complex, Vitamin B1, Vitamin B2, Vitamin B3, Vitamin B5, Vitamin B6, Vitamin B7, Vitamin B9, Vitamin B12, inositol, 4-aminobenzoic acid, folinic acid, and/or Vitamin E.

### EXPERIMENTAL EXAMPLES

### Example 1. Experimental Methods

Ethical statement, animal use, breeding, care, and euthanasia criteria for survival studies. All care of experimental animals and experiments were performed as approved by the Institutional Animal Care and Use Committee.

Ndufs4(+/−) mice were bred to produce Ndufs4(KO) (Ndufs4(−/−)) offspring. Mice were weaned at 20-21 days of age. Ndufs4(KO) animals were housed with a minimum of one control littermate for warmth and stimulation. Mice were weighed, and health assessed, a minimum of 3 times per week, every other day (daily for IP injected mice, see herein). Where longitudinal blood point-of-care data was collected, this was performed during these health checks. Wetted chow in a dish on the bottom of each cage, and in-cage water bottles, were provided to all cages housing Ndufs4(KO) mice following the onset of disease so that the ability to find or reach food or water was not a limiting factor for survival. Mice were euthanized if they showed a 20% loss in maximum body weight, immobility, or were found prostrate or unconscious.

As previously reported, Ndufs4 deletion is a recessive defect, and heterozygosity results in no reported or observed phenotypes, including no detectable defects in electron transport chain complex I (ETC CI) activity, so controls for this dataset include both heterozygous and wildtype mice.

All experimental mice were fed PicoLab Diet 5058; pharmacologic agents were compounded into this diet (described herein).

Longitudinal assessments of neurological disease symptoms. Clasping, ataxia, and circling were assessed by visual scoring, as previously described. As disease progresses in the Ndufs4(KO) mice animals display intermittent/transient improvement of symptoms. For these studies, the analysis was simplified to report whether animals had ever exhibited the symptom—requiring that the symptom was observed for two or more consecutive days to minimize spurious reporting. Transient loss of symptoms was not included in these data. For observational assessments, lab-wide quality control discussions occurred frequently to ensure consistency between technicians/researchers. Technicians contributed equally to each treatment group to minimize any potential bias between individuals.

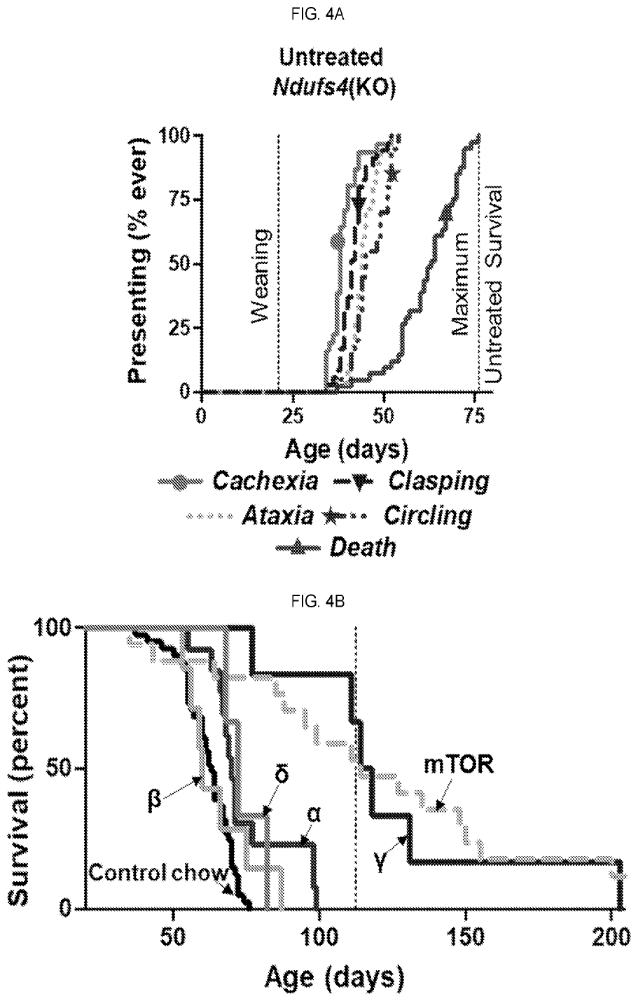

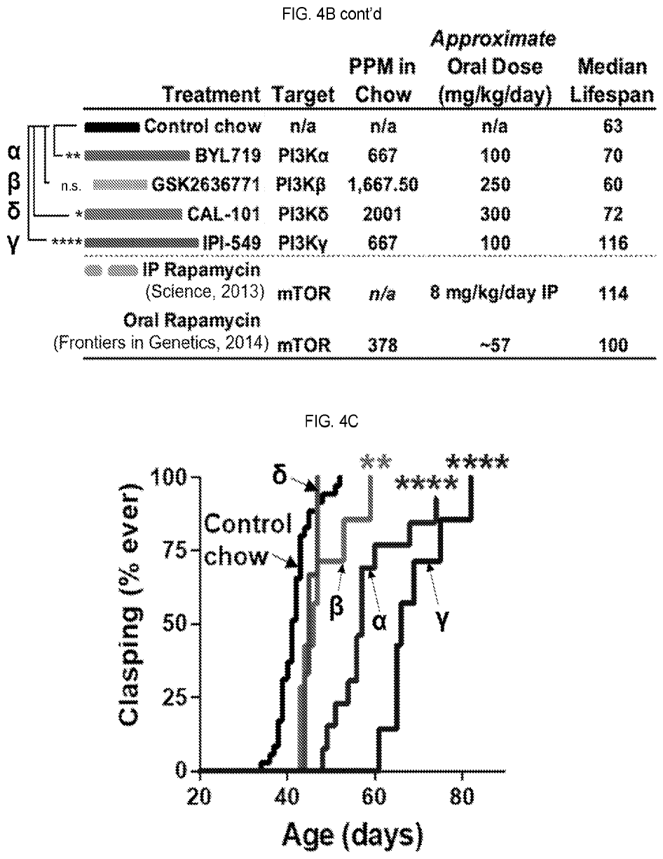

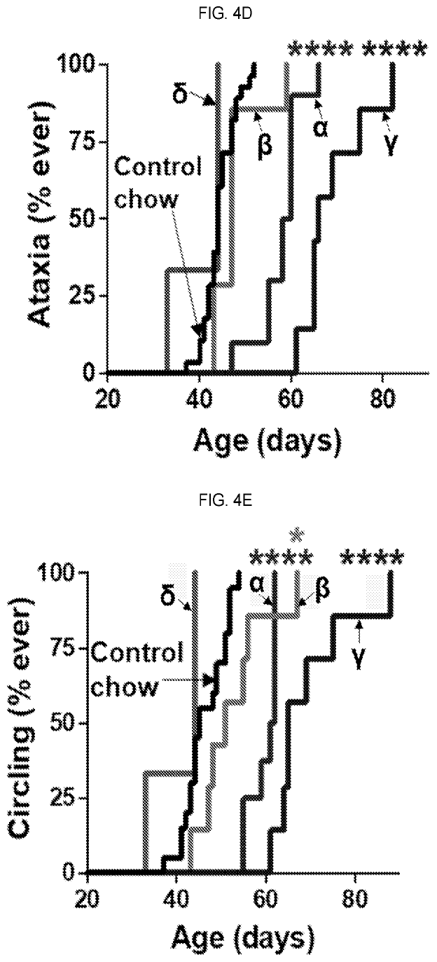

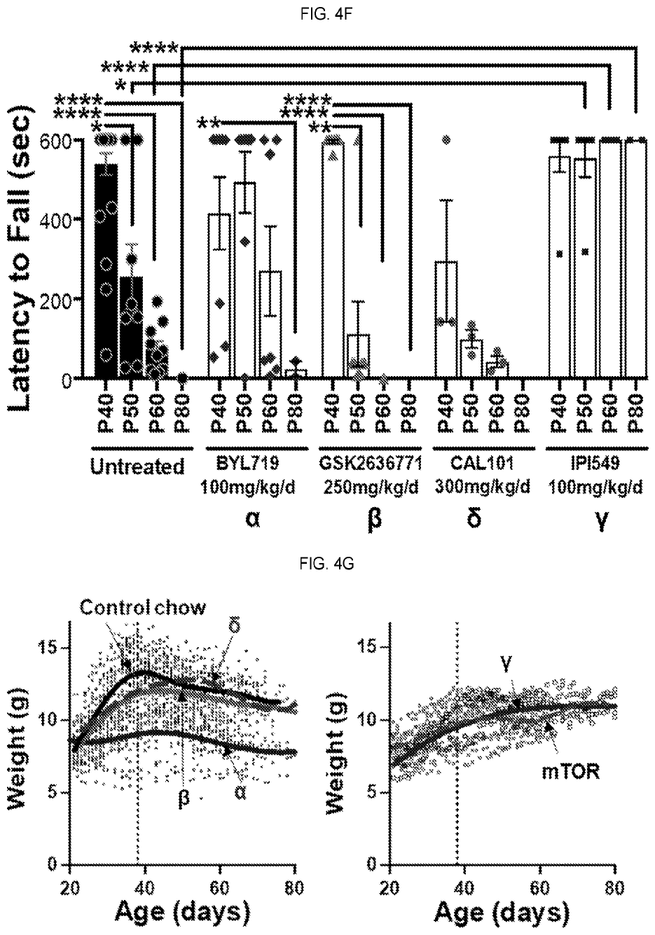

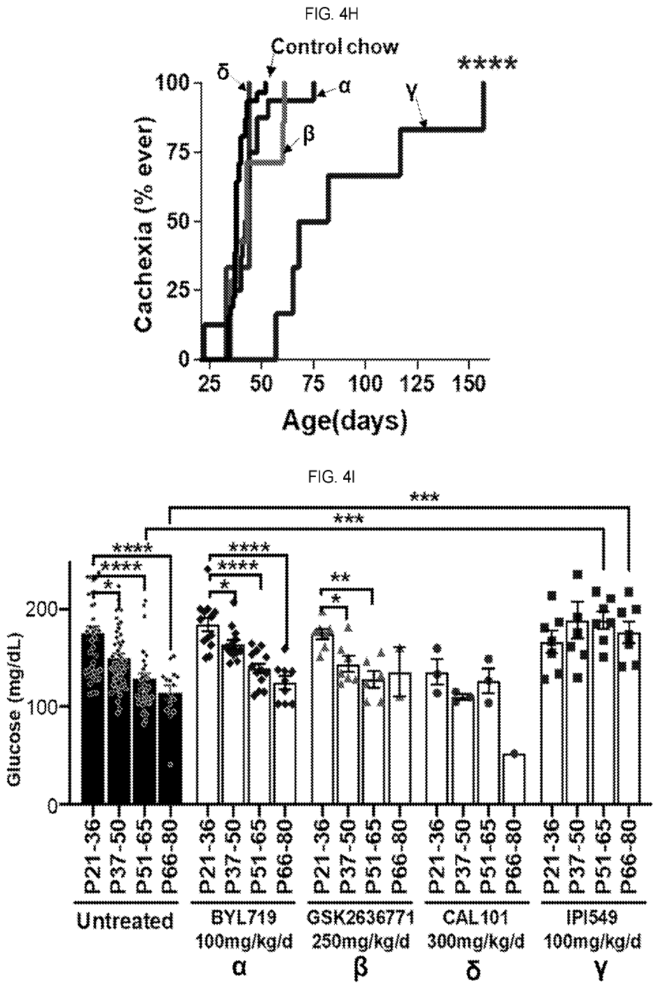

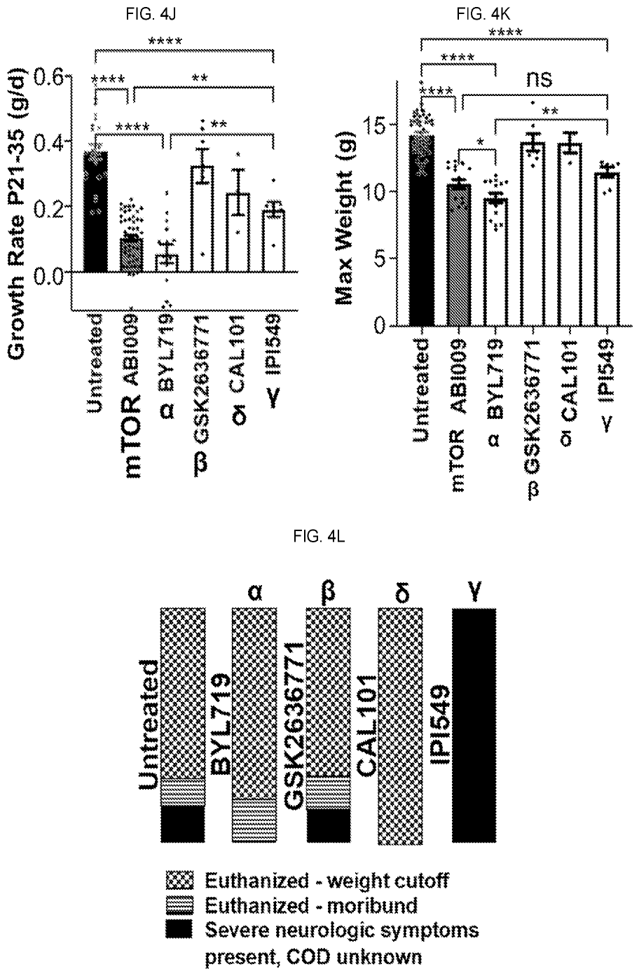

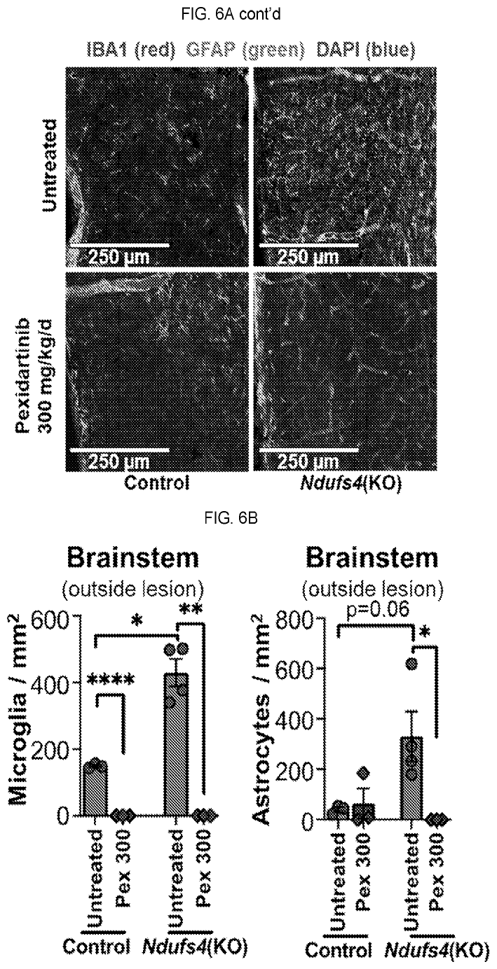

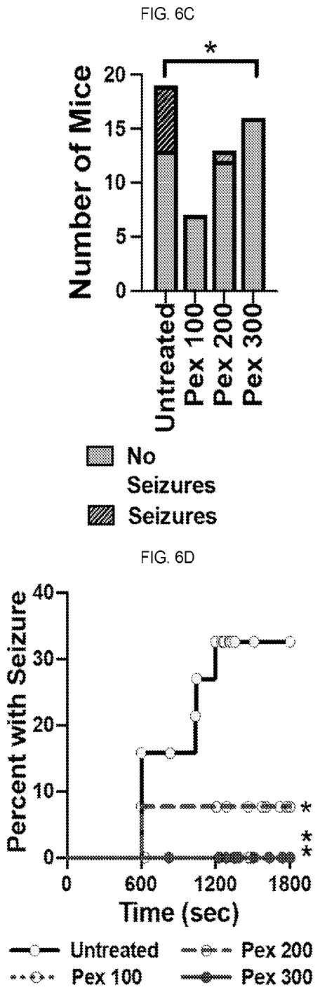

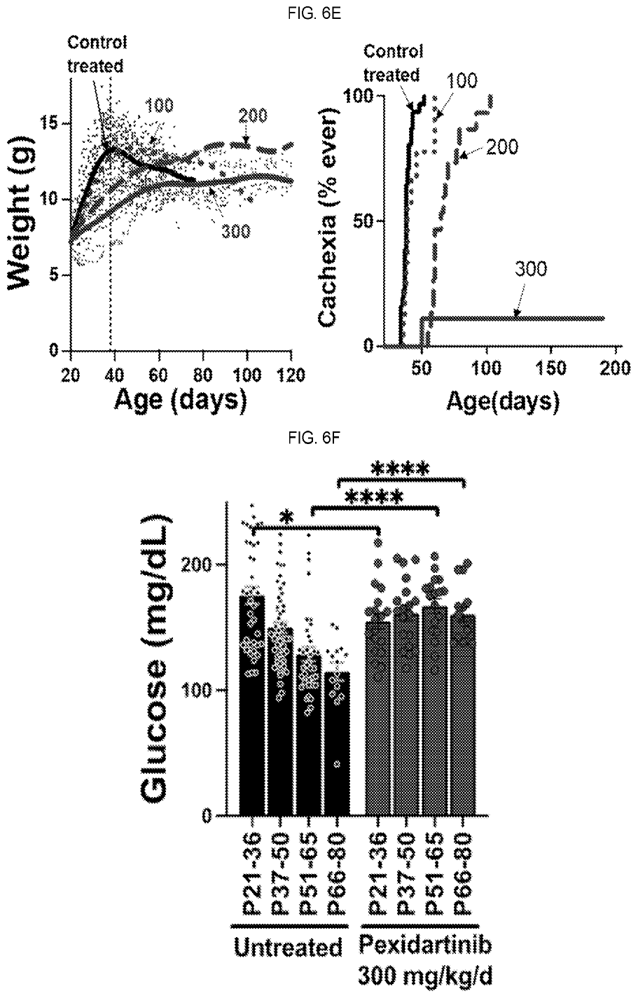

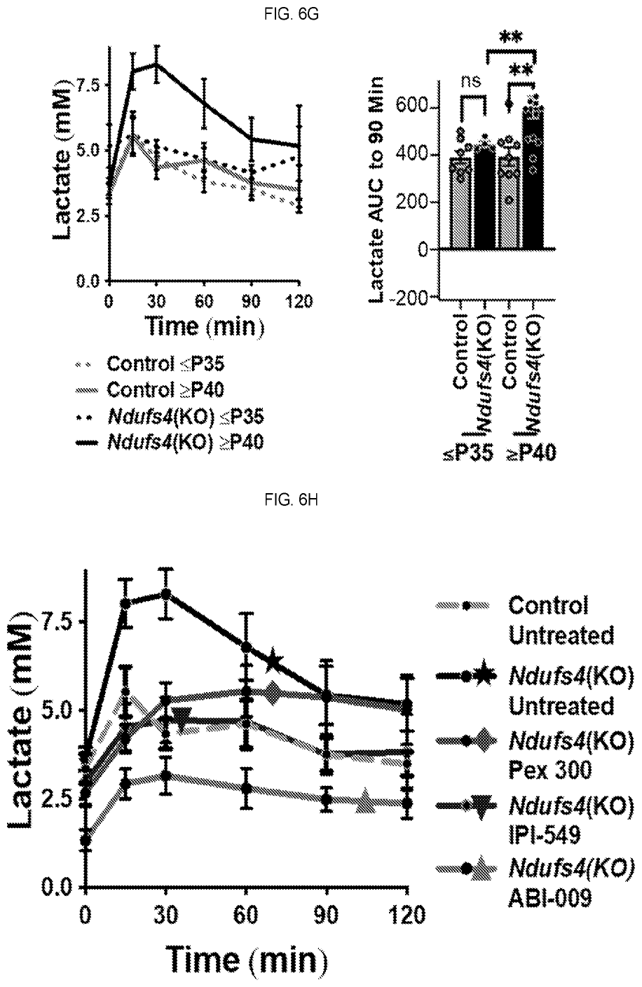

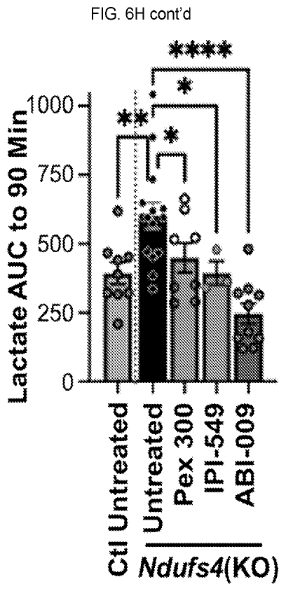

Cachexia in FIG. 4H and FIG. 6E is the day of life when weight peaks prior to the progressive weight loss which occurs in untreated Ndufs4(KO) animals.

Replicate numbers and control group. Animal numbers for each dataset are noted in figure legends, and whenever possible all individual datapoints are shown. Animals were added to the control treatment groups across the duration of the experiments presented here to ensure that no shifts in colony survival, behavior, etc., occurred during the course of these studies. Accordingly, control treatment groups generally contain larger cohorts than the individual treatments.

Reagents. Laboratory chemicals were purchased from Sigma. Pexidartinib, IPI-549, CAL-101, and GSK2636771 were purchased from MedKoo Biosciences (cat. #'s 206178, 206618, 200586, and 205844, respectively). BYL719 was purchased from AChemBlock (cat. #R16000).

Doses of each drug was based on published data for use in animals. If no effect was observed at published dosing, dosage was increased until either positive effects or toxicity were observed. Blood and tissue concentrations reached biologically relevant levels for each of the tested inhibitors.

Pharmacologic interventions and chow formulation. Standard mouse chow was ground to a power and mixed with drugs at the doses desired (see FIGS. 4A-4L). 300 mL of 1% agar melted in sterile water was added per kilogram of powdered chow and the mixture was pelleted and baked at 37° C. for 3-5 hours to dry. Pellets were then stored at 4° C. for short term (up to 90 days) or −20° C. for long-term (up to 6 months) storage. It was previously shown that this processing had no impact on animal health or survival (Johnson et al. Front Genet. 2015; 6:247).

ABI-009. Rapamycin was provided in the form of ABI-009 (nab-rapamycin), an albumin encapsulated water-soluble formulation. ABI-009 was provided by Aadi Bioscience, LLC (Los Angeles, CA) in lyophilized form. ABI-009 was resuspended to 1.2 mg/mL rapamycin in 1× phosphate-buffered saline (PBS). This solution was sterile filtered and stored in aliquots at −80° C. ABI-009 was administered at 66 μL per 10 g for a final dose of 8 mg/kg/day rapamycin, as in prior studies (Johnson et al. Front Genet. 2015; 6:247; Johnson et al. Science. 2013; 342(6165):1524-1528).

Point-of-care glucose and lactate testing. Blood glucose and lactate measurements were collected using point-of-care meters (Prodigy Autocode® glucose meter, product #51850-3466188, Prodigy® Diabetes Care, LLC, Charlotte, NC; lactate assay meter, product #40828, Nova Biomedical, Waltham, MA) and the tail-prick method, as previously described (Stokes et al. Elife 2021; 10:e65400).

Rotarod and rotarod seizures. Rotarod assays were performed using a Med Associates Inc. (Fairfax, VT) ENV-571M single lane rotarod with a black mat installed in the floor of the lane to reduce visibility of the bottom. Assays were performed by placing animals onto an already rotating rod and timing latency to fall, with a steady rotation speed set to 6 rpm (controlled by attached laptop and Med Associates software). The maximum time of each trial was 10 min, with the trial ending at that time if mice were still on the rotarod. For each assay, three trials were performed, with a minimum of 5 min between each assay. The best of the three trials was reported for the rotarod data. As detailed in the text and figure legends, rotarod was performed each 10 days of age from P30; mice in a given age group were the reported age +/−1 day.

Mice were monitored throughout each assay for seizure activity, and rotarod performance trials were ended if a seizure was observed. Mice were considered to be showing seizure activity if any symptoms on the Racine behavioral scale or Pinel and Rovner scale were observed: abnormal oroalimentary movements (dropping of the jaw repeatedly, atypical gnawing or chewing movements), repeat head nodding, anterior limb clonus (twitching/jumping while making no contact with the face), dorsal extension/rearing, loss of balance and falling (observable only after mouse has exited the rotarod), violently running/jumping (‘popcorning’). Critically, no attempt was made to assess seizures on the seizure scales for these studies—only presence or absence was noted.

While blinding was often impossible (BYL719 treatment leads to extremely small body size, pexidartinib turns fur white, and overtly diseased Ndufs4(KO) mice are easily identified), the experiments were scored by technicians with no expectations regarding the outcomes. Technicians trained together and cross-compared observed symptoms to ensure consistent scoring, and analysis of each treatment group was roughly evenly split among technicians involved in these studies to prevent any unaccounted-for observer bias from impacting any individual treatment group.

Glucose tolerance test (GTT) and glucose induced lactate test. Prior to performing the GTT, mice were fasted for 4 hours, from 10 AM-2 PM. The fasting duration was limited to 4 hours given the sensitivity of untreated Ndufs4(KO) animals to hypoglycemia and related sequelae (dysregulation of body temperature, hypoglycemia induced seizure, etc.). For the ABI-009 cohort, mice received their daily ABI-009 treatment at the start of this fasting period. At the end of the 4 hour fast, baseline blood glucose and lactate values were collecting using point-of-care meters and the tail-prick method. Mice were then injected by IP with 2 g/kg dextrose (10 microliters/gram body weight of 200 mg/mL glucose in 1×PBS, 0.2 micron sterile filtered) using an insulin syringe (31-gauge, 6 mm length, 3/10 cc, BD Veo Ultra-Fine needle). Glucose and lactate levels were measured using a minimally invasive tail-prick method at designated timepoints post-injection.

Isoflurane induced hyperlactemia assays. All of these experiments were performed at P50. Mice were subject to normal daily monitoring (see survival studies) up until P50. At P50, baseline blood glucose, lactate, and β-hemoglobin (β-HB) levels were measured using point-of-care meters using the tail-prick method, as described. Mice were then immediately exposed to 0.4% isoflurane or carrier gas only (100% oxygen) for 30 min. At the 30 minutes, blood glucose, lactate, and β-HB were measured again.

Respiratory measurements. Breathing parameters were recorded from alert, unrestrained, adult (P60) mice using whole-body plethysmography. Paired 300 ml recording and reference chambers were continuously ventilated (150 ml/min) with either normal air (79% nitrogen and 21% oxygen) or a hypercapnic gas mixture (74% nitrogen, 21% oxygen, and 5% CO₂). Pressure differences between the recording and reference chambers were measured (Buxco Respiratory Products) and digitized (Axon Instruments) to visualize respiratory pattern, and simultaneous video recordings were performed to differentiate resting breathing activity from exploratory sniffing and grooming behaviors. Untreated and treated (pexidartinib 300 mg/kg/day) control and Ndufs4(KO) mice were allowed to acclimate to the chambers for 30-40 min prior to acquisition of 35 min of respiratory activity in normal air. The respiratory response to hypercapnia was then tested by ventilating the chambers with hypercapnic gas for 15 min. Respiratory frequency, and breath-to-breath irregularity scores (irreg.score=ABS((N-(N−1))/N)) of frequency and amplitude (peak inspiratory airflow) were quantified during periods of resting breathing (pClamp10 software). During hypercapnic challenges, only data during the final 5 min of the 15 min period were analyzed. Results were visualized and statistical comparisons between groups were performed in GraphPad Prism9 software.

Mixed brain cell culturing and staining. Briefly, using sterile technique, brains were removed from neonatal pups into ice-cold HBSS (Gibco cat. #14025076). Brains were washed three times in cold PBS. After the third wash, brains were minced using surgical scissors into small, fine pieces. The minced brain was centrifuged at 100 g for 5 min. Once pelleted, PBS was removed and 0.05% trypsin/EDTA (Gibco) was added. The tissue in trypsin/EDTA was then incubated at 37° C. with gentle shaking for 15-30 min, with intermittent mixing and supplemental mincing, until the solution was homogenous and without tissue segments. After incubation, the minced tissue was placed back into the centrifuge at 100 g 5 min. Pellet was washed 3× with cold PBS before adding fresh media to each tube. The tissue was further dissociated using 1000 μL pipet until the solution became cloudy. The sample was washed through a sterile 70-micron cell strainer (Corning) into a 50 mL falcon tube, then plated onto T75 poly-d-lysine coated Nunclon flasks (Nunc EasyFlask132704) at a ratio of one brain to two flasks. After 24 hours, dead cell/debris were washed and media replaced. Cells were then grown for 1 additional day, split onto multi-well plates with poly-d-lysine coated coverslips (Cellware 12 mm round coverslips) at 25% confluence with ABI-009 or pexidartinib (for dose-response) added. Media was replaced (with pharmacologic agents) every 3 days, and cells were fixed in ice-cold 3.7% PFA on day 7.

Fixed cell slides were washed 3× with 1×PBS followed by a 5 min incubation in 0.2% Triton-X100/1×PBS at room temperature. Slides were washed again 3× in 1×PBS, blocked for 30 min at room temperature in 10% rabbit serum, and then incubated in antibody solution overnight at 4° C. protected from light. Antibody solution consisted of blocking solution with rabbit anti-Iba1-635 (Wako, cat. #013-26471) at 1:400 and DAPI (Sigma, cat. #D9542) at 10 μg/mL.

Cell media for these experiments consisted of 500 mL of DMEM (Gibco ref #11995-065), 56.2 mL of One-Shot Fetal Bovine Serum (FBS) (Gibco cat. #16000-077), and 5.62 mL of penicillin/streptomycin, 10,000 U/mL (Gibco cat. #15140122).

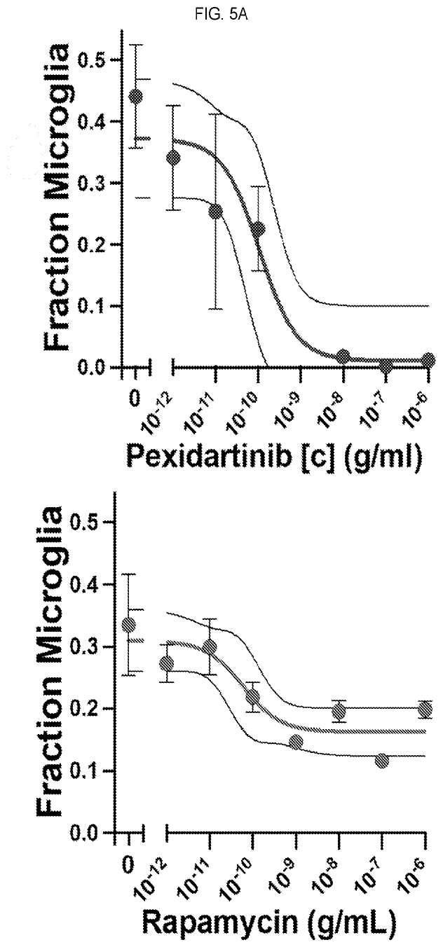

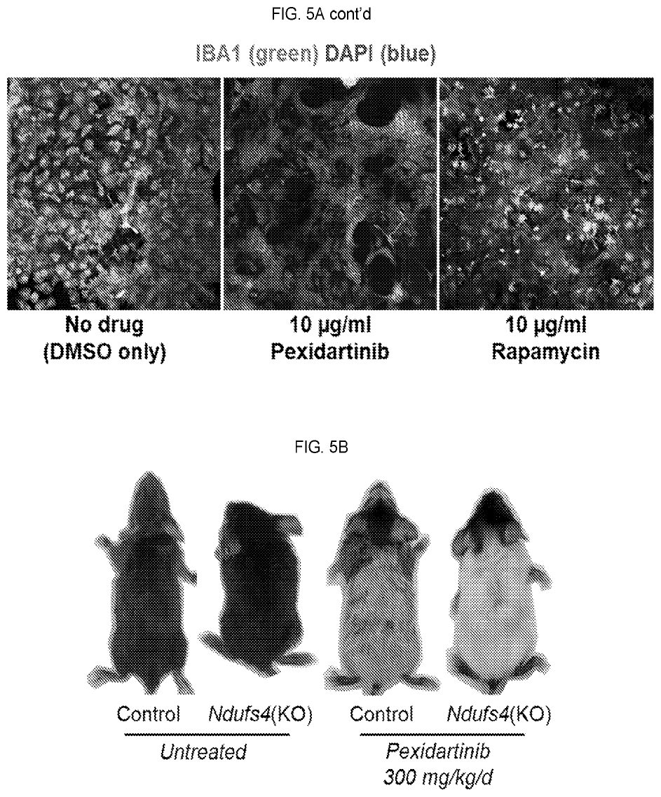

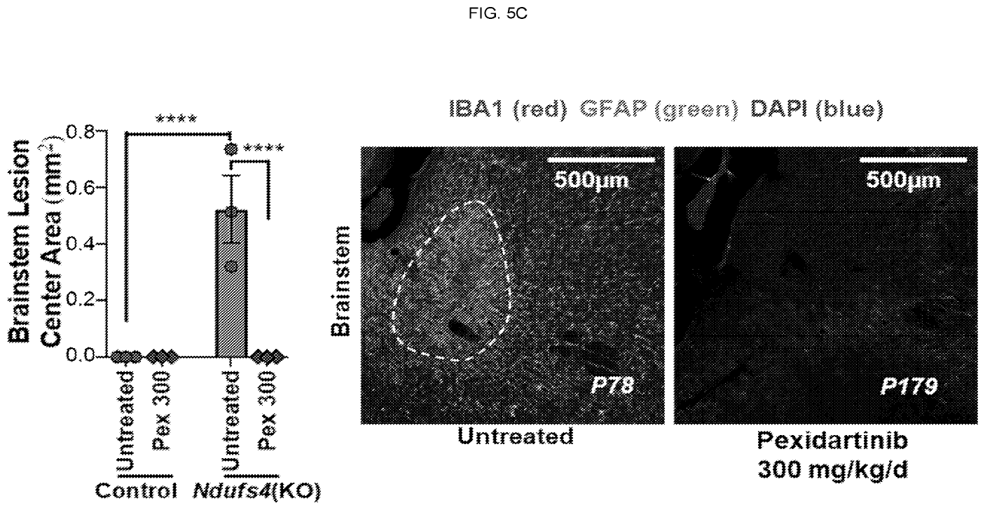

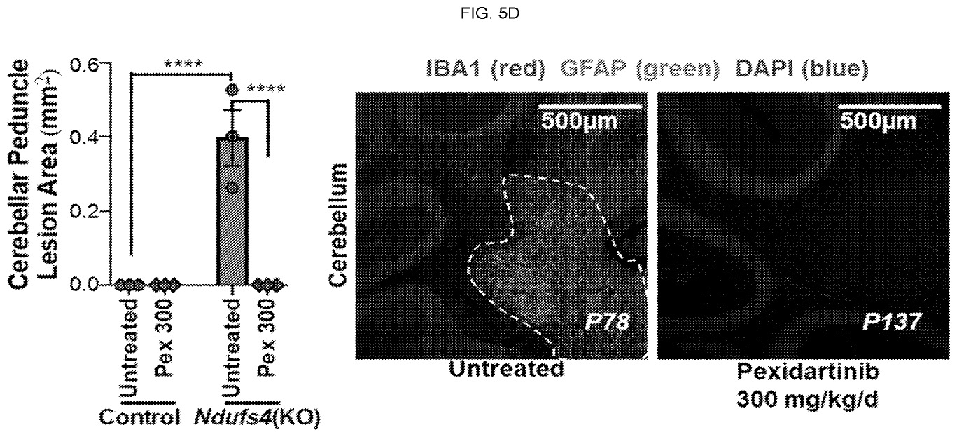

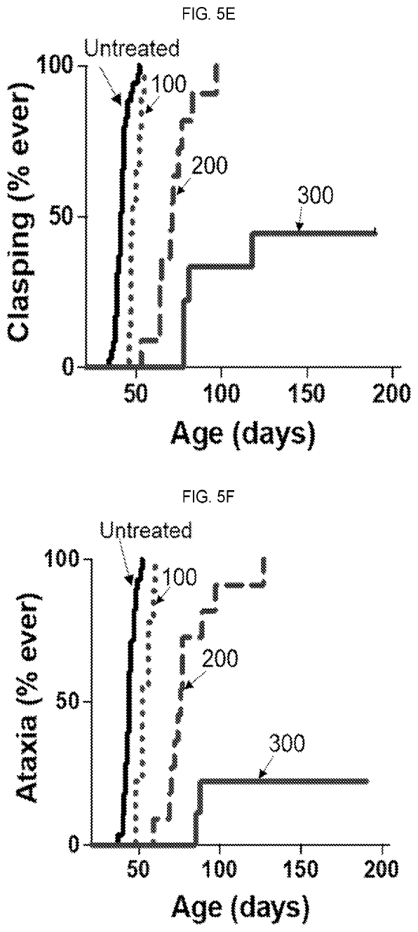

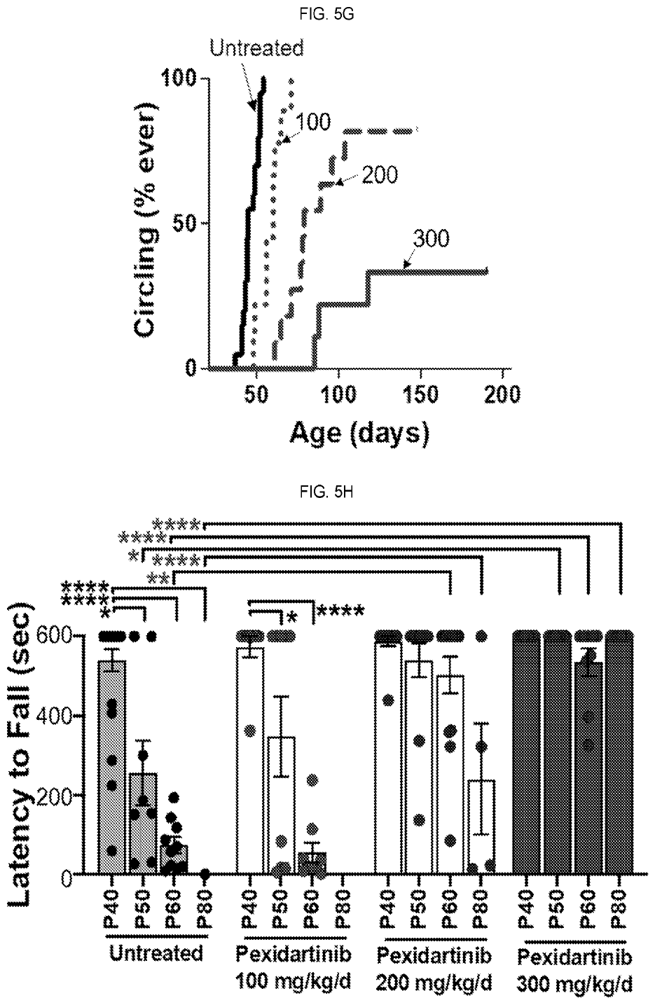

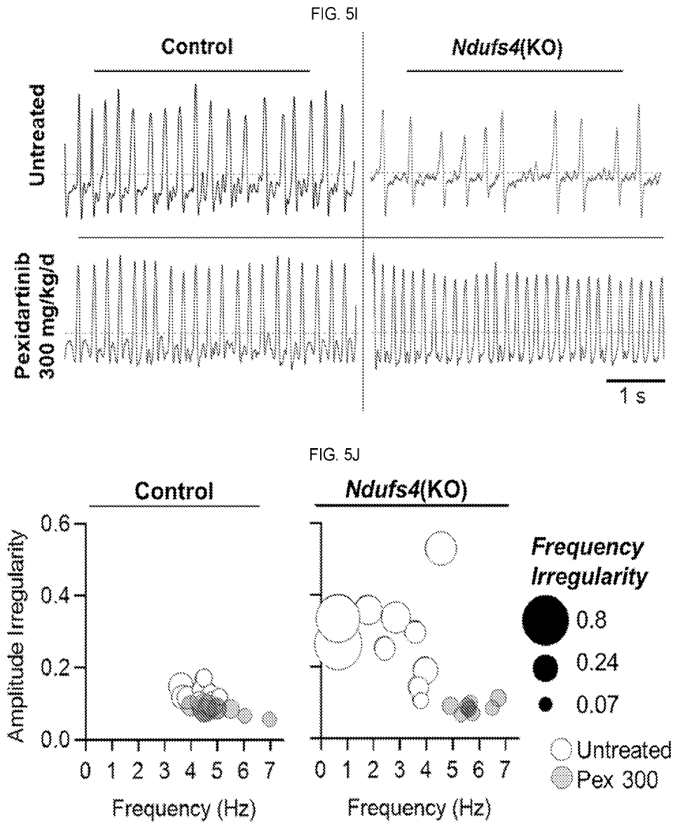

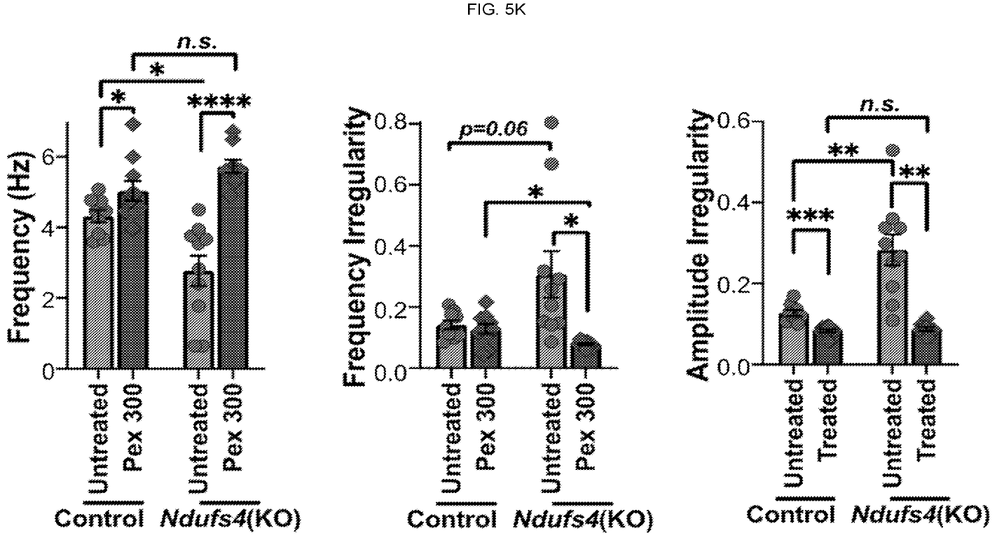

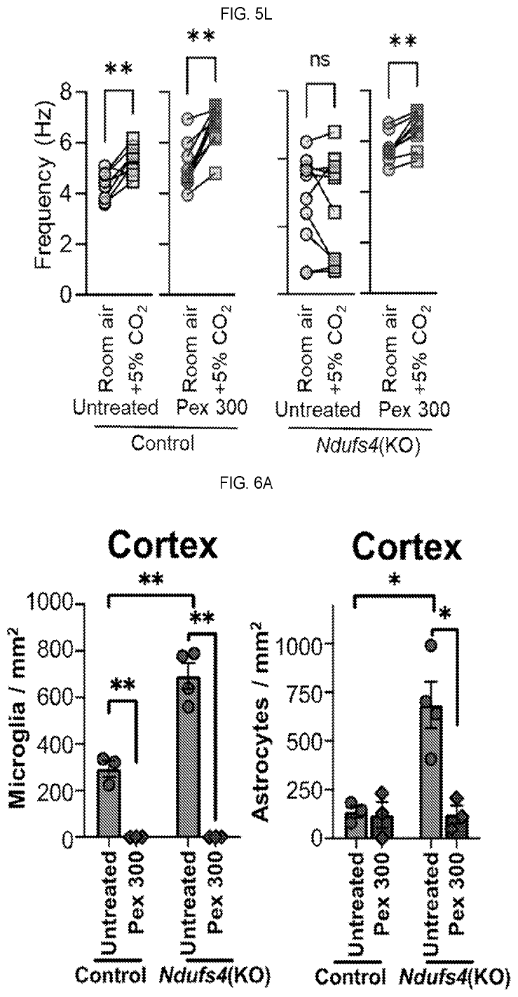

Pharmacologic agents were sourced as described herein, dissolved to 1000× in DMSO, and added at 1:1000 to media for working media. DMSO was added to the ‘no drug’ wells. These experiments were repeated 3 times with similar results. Data in FIGS. 5A-5L were derived from biological replicates from the same experiment.

Brain immunological staining and microscopy. Brains were fixed for 48 hours in 10% formalin at 4° C. Following fixation, brains were moved to a cryoprotectant solution (30% sucrose, 1% DMSO, 100 μM glycine, 1×PBS, 0.45 μm filtered, pH 7.5), and stored for over 48 hours, until the fixed tissues sank to the bottom. Tissues were then placed in OCT media (Tissue-Tek OCT compound, Sakura 0004348-01), frozen in cryoblock holders on dry ice, and stored at −80° C. until sectioned for staining. Cryoblocks were cut at 50 μm thickness using a Leica CM30505 cryostat set at −40° C. Slices were moved to 1×PBS and stored at 4° C. until used for staining. Prior to staining, slices were mounted on slides and briefly dried to adhere.

Antibody staining was performed as follows: slides were first incubated in an antigen retrieval and permeabilization buffer (0.05% Triton X-100, 50 μM digitonin, 10 mM Tris-HCl, 1 mM EDTA, pH 9.0) at 60° C. overnight in a white-light LED illuminated box (to promote photobleaching of tissue autofluorescence). To reduce formaldehyde induced background fluorescence, slides were treated with sodium borohydride in ice cold PBS, added at 1 mg/mL immediately before incubation, on ice for 30 min, then moved to 10 mM glycine 1×PBS, pH 7.4, for 5 min at room temperature. Lipid background fluorescence was then blocked by incubating slides in 0.2 μm filtered Sudan Black B solution (5 mg/mL in 70% ethanol) overnight at room temperature. Slides were then rinsed twice, 5 min each, in 1×PBS. Excess fluid was wiped from the slide, and the tissue was circled using Liquid Blocker PAP pen (Fisher Scientific, NC9827128) to hold staining solutions. Slides were blocked for 15 min at room temperature in 1×PBS with 10% rabbit serum (Gibco, 16120-099) then stained overnight at 4° C. in a mixture of rabbit anti-IBA1-fluorochrome 635 conjugated (WAKO 013-26471) at 1:300, mouse anti-GFAP, Alexa555 conjugated (Cell Signaling, 3656S) at 1:300, and DAPI (Sigma, D9542) at 1 μg/mL. The following day, slides were washed 3×5 min in 1×PBS then mounted in aqueous mounting media (Fluoromount-G), coverslipped, and stored at 4° C. protected from light until imaging.

Slices were imaged on a Zeiss LSM 710 confocal microscope. Images were collected using a 10× dry objective at 0.6× optical zoom, resulting in images of 1417×1417 microns in physical area. Images were collected with 8-16 line averages and a line scan speed of 6-7. Channels were set to an optical thickness of 50 nm. DAPI was excited at 405 nm, with emission light collected using a sliding filter with the range setting at 424-503 nm. GFAP-Alexa555 was excited with a 543 nm laser, emissions collected at 548-587 nm. Iba-1-635 was excited at 633 nm and emitted light was collected at 641-661 nm.

Plasma ALT and AST measures. Small volumes (5-10 μL) of blood were collected using tail-prick and heparinized microhematocrit tubes (Fisher Scientific cat. #22-362566), placed on ice in 15 mL sample tubes, then moved (by pipette) into 1.5 mL microcentrifuge tubes and stored at −80° C. until used for ALT and AST enzymatic assays. ALT and AST were quantified using colorimetric enzymatic activity assay kits (Sigma, cat. #'s MAK052 and MAK055, respectively) according to manufacturer recommendations. Absorbance was measured on a NanoDrop 1000 (ThermoScientific).

Chemokine analyses. Brainstem chemokines were analyzed by Eve Biotechnologies using the Millipore MCYTMAG-70K-PX32 Milliplex MAP Mouse Cytokine/Chemokine Magnetic Bead Panel Multiplex panel. Brainstem samples were collected rapidly after euthanasia, flash frozen in liquid nitrogen, and shipped to Eve Biotechnologies on dry ice. Samples were processed and analyzed according to Millipore manufacturer recommendations.

Statistical analyses. All statistical analyses were performed using GraphPad Prism as detailed in figure legends. Unless otherwise stated, error bars represent standard error of the mean (SEM), and p<0.05 is considered statistically significant.

Example 2. Genetic mitochondrial diseases (MDs) are the most common cause of heritable metabolic disease and one of the most common causes of pediatric neurological dysfunction. MDs are genetically and clinically heterogeneous, with cases clustering into clinically defined syndromes. Subacute necrotizing encephalopathy, or Leigh syndrome (LS), is a multi-organ disease with metabolic, neurologic, and musculoskeletal symptoms, and is the most common form of pediatric MD. LS patients are often born healthy, showing symptom onset within the first few years of life. Symmetric progressive necrotizing lesions in the brainstem are a defining feature of LS, but a mechanistic understanding of these lesions has been elusive, and no effective interventions exist in the clinic. Inhibition of the mechanistic target of rapamycin (mTOR) attenuates disease in the Ndufs4(KO) mouse model of LS, and mTOR inhibition appears to benefit some patients, but the exact mechanisms of mTOR inhibition in MD have been elusive. Using small-molecule testing in the Ndufs4(KO) model, the present study found that benefits of mTOR inhibitors can be attributed to inhibition of signaling mediated by the PI3K catalytic subunit gamma isoform, p110γ/PI3Kγ, known to be predominately expressed in leukocytes. Directly targeting leukocyte proliferation through inhibition of Colony Stimulating Factor 1 Receptor, CSF1R, with pexidartinib dramatically attenuates disease in the Ndufs4(KO) mice. CSF1R inhibition blocked CNS lesion formation, prevented neurologic symptoms, and extended survival. Strikingly, CSF1R inhibition also rescued symptoms not yet directly tied to CNS lesions, including hyperlactemia, seizures, and anesthetic responses. Critically, pexidartinib treated Ndufs4(KO) animals live as long as treated control mice, with drug toxicity, rather than MD, appearing to limit lifespan. Together, these findings provide evidence for the mechanism of mTOR inhibition in attenuating LS and reveal that many symptoms of this disease have an immunologic origin amenable to direct pharmacologic targeting. These findings are of significant importance to understanding of the pathogenesis of genetic MD and may lead to novel therapeutic strategies.

Isoform specific pharmacologic targeting of PI3Kγ significantly attenuates disease in the Ndufs4(KO) mouse model of LS. To determine whether the benefits of mTOR inhibition in Ndufs4(KO) model result from disruption of PI3K mediated signaling, animals were treated from weaning (post-natal day 21, P21) with potent, orally available, isoform specific inhibitors of the catalytic subunits of PI3K: BYL719, GSK2636771, CAL-101, and IPI-549, inhibitors of p110α, p110β, p110δ, and p110γ, respectively.

Control diet fed Ndufs4(KO) animals had normal health early in life but displayed rapidly progressive neurological symptoms associated with CNS degeneration beginning around P37; death occurred by P80 (FIG. 4A). Treatment with the p110α, p110β, and p110δ inhibitors at doses leading to bioactive drug levels in blood and tissue provided no benefit to disease or survival (FIGS. 4B-4L). Treatment with BYL719, the p110α inhibitor, led to a statistically significant delay in symptom onset and extension of survival, but the magnitude of the effect was modest and likely due to an overall delay in development. In contrast, treatment with the p110γ inhibitor IPI-549 markedly extended survival and attenuated disease (FIGS. 4B-4L). IPI-549 significantly delayed the onset of forelimb clasping, ataxia, and circling; improved Ndufs4(KO) performance on a rotarod assay (which assessed neurologic and muscular function and overall health); and extended survival (FIGS. 4B-4F). The impact of p110γ/PI3Kγ inhibition on survival was strikingly similar to mTOR inhibition—median survival in the IPI-549 treated Ndufs4(KO) mice was 110 days, versus 110 and 60 for rapamycin treated and untreated animals, respectively (FIG. 4B).

In addition to attenuating neurologic sequelae, IPI-549 prevented the cachexia and progressive hypoglycemia associated with disease progression in the Ndufs4(KO) (FIGS. 4G-4I) (metabolic endpoints are further discussed herein).

Treatment with rapamycin at doses sufficient to attenuate disease resulted in significant reductions in developmental weight gain and maximum body size (Johnson et al. Front Genet. 2015; 6:247; Johnson et al. Science. 2013; 342(6165):1524-1528). The impact of IPI-549 on disease was similar to mTOR inhibition (FIG. 4J). In contrast, BYL719 severely impaired growth and size, significantly more than mTOR inhibition, while only modestly impacting disease (FIGS. 4J, 4K). Furthermore, the slight shift in disease onset may simply be due to developmental delay (delay in normal P35 weight peak), rather than any impact on MD pathogenesis per se. Together, the BYL719 and IPI-549 data suggest mTOR inhibition does not benefit MD through actions on insulin/IGF-1 signaling (IIS) as p110α, and not p110γ, mediates IIS.

BKM-120, a pan-PI3K inhibitor, was also tested. While well-tolerated in adult mouse models at up to 60 mg/kg/day (Burger et al. ACS Med Chem Lett. 2011; 2(10):774-779), BKM-120 was not tolerated at 50 or 100 mg/kg/day when started at weaning.

mTOR inhibition reduces microglial proliferation in vitro. p110γ is primarily expressed in leukocytes, which include brain resident microglia. Lesions in LS are characterized in part by microgliosis, widely assumed to be secondary to tissue necrosis caused by CNS cell death. Given that p110γ and mTOR inhibitors provided similar benefits, while p110α, p110β, and p110δ inhibitors failed to alter disease, leukocyte (including microglia) proliferation may drive CNS degeneration and lesion development, rather than occurring as a response to CNS damage. In this model, mTOR and PI3Kγ might attenuate disease through directly impairing leukocyte proliferation. Consistent with this notion, both ABI-009 (a water-soluble formulation of rapamycin) and pexidartinib (a CSF1R inhibitor) dose-dependently reduced the fraction of microglia present in mixed neonatal brain-cell cultures (FIG. 5A). Notably, the maximum effect size of ABI-009 was only 50% of total compared to a nearly complete depletion of microglia with pexidartinib, which directly inhibits leukocyte survival signaling. Accordingly, the potency of mTOR inhibition appears limited compared to direct targeting of leukocyte survival through CSF1R.

Leukocyte depletion prevents CNS lesions and associated neurologic sequalae, including respiratory failure. Gliosis at the site of CNS lesions in LS has been viewed as a response to tissue degeneration; a role for immune cell proliferation in driving disease in LS has not been reported. However, the PI3K and in vitro findings indicated that mTOR inhibitors benefit the Ndufs4(KO) through their inhibitory effects on leukocyte proliferation, suggesting that leukocytes (including microglia) may be directly mechanistically involved in the pathobiology of LS. If so, reigning in leukocyte proliferation should attenuate the disease.

To directly test this model, Ndufs4(KO) and control animals were treated with 100, 200, or 300 mg/kg/day pexidartinib in normal mouse chow (dosing is approximated based on food consumption, see Experimental Methods in Example 1). The higher doses led to a change in mouse coat color, consistent with reports of hair whitening in humans (FIG. 5B).

Treatment with 300 mg/kg/day pexidartinib led to a complete (as detected by immunohistochemistry) prevention of brainstem (FIG. 5C) and cerebellar (FIG. 5D) CNS lesions in Ndufs4(KO) mice, even at ages far beyond the maximum survival of untreated animals (see herein for survival data). Critically, both forms of gliosis (microgliosis and astrocytosis) were completely prevented by pexidartinib. Notably, the rescue of astrocytosis by a leukocyte inhibitor suggested that astrocyte involvement is secondary to leukocyte/microglia activity, a detail of LS pathobiology not previously resolved.

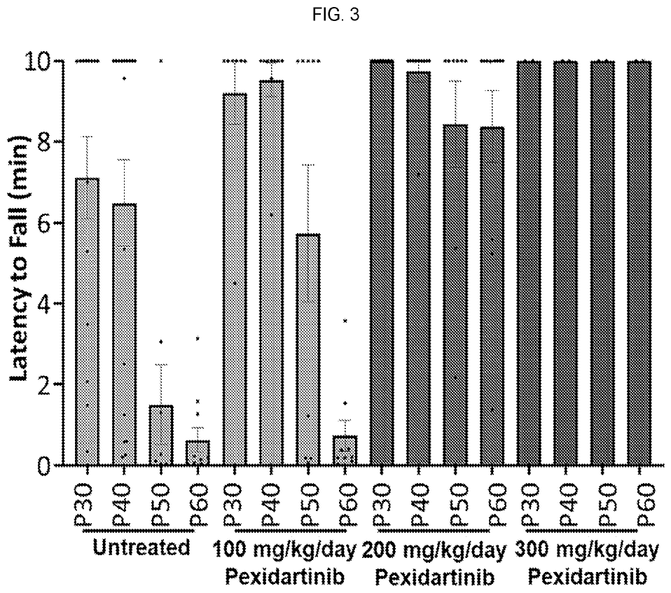

Consistent with these histological findings, pexidartinib dose-dependently delayed the onset and reduced overall incidence of behavioral signs of brainstem and cerebellar degeneration—forelimb clasping, ataxia, and circling (FIGS. 5E-5G). In each of these endpoints, the 200 mg/kg/day dose roughly matched mTOR inhibition, while 300 mg/kg/day dose performed substantially better. Pexidartinib treatment also rescued performance on the rotarod assay (FIGS. 3 and 5H).

Impaired respiratory center activity, which is a brainstem function, is a proximal cause of death in LS patients and Ndufs4(KO) mice. To determine whether respiratory center function is rescued by pexidartinib, plethysmography analysis was performed in untreated and 300 mg/kg/day pexidartinib treated mice. These experiments confirmed that severe defects in the control of breathing function are present in untreated Ndufs4(KO) mice by P60, while, remarkably, treatment with 300 mg/kg/day pexidartinib completely rescued respiratory dysfunction in normal air and the respiratory response to increased CO₂ (FIGS. 5I-5L).

Overt CNS lesions in the brainstem and cerebellum underlie many of the defining features of LS, but evidence from AIFM1 (apoptosis-inducing factor, mitochondria-associated, 1) deficient mice, which are a milder model of MD, suggests that microgliosis may be widespread in MD brains, and that inflammation outside of overt lesions may contribute to symptoms (Wischhof et al. Mol Metab. 2018 July; 13: 10-23). Given the robust impact of pexidartinib in preventing lesions, other brain regions were studied to assess whether neuroinflammation was present, and responsive to pexidartinib, in other Ndufs4(KO) brain regions. Analysis of cortex and brainstem tissue outside of the overt lesions revealed that both astrocytosis and microgliosis were indeed present in non-lesion CNS tissue in the Ndufs4(KO) mice, and that 300 mg/kg/day pexidartinib prevented both forms of gliosis (FIGS. 6A, 6B). Notably, pexidartinib treatment led to a near complete depletion of microglia, beyond that of untreated control animals, while astrocyte numbers were simply rescued to control levels, again indicating that astrocytosis is mechanistically downstream of leukocyte involvement. As with lesions, inflammation was prevented by pexidartinib treatment even in brains collected from mice far older than the maximum lifespan of untreated Ndufs4(KO) animals (see FIGS. 6A-6I legend for details).

Pexidartinib treatment prevents rotarod induced seizures. Epileptic seizures are common in LS and can be intractable to standard therapies. The physical activity and any associated stress in the rotarod assay provided a mild epileptogenic stimulus: seizures occurred during the rotarod assay at a frequency of 30% in untreated Ndufs4(KO) mice at age P30 (FIG. 6C). Seizures were never observed in control animals. To determine whether pexidartinib attenuated seizures, additional rotarod experiments were performed on control and pexidartinib treated Ndufs4(KO) mice and monitored epileptic activity. Incidence of rotarod induced seizures, and time-to-seizure, were both markedly reduced by pexidartinib (FIGS. 6C, 6D).

Pexidartinib treatment rescues hypoglycemia and cachexia, and hyperlactemia is prevented by rapamycin, IPI-549, and pexidartinib. Treatment with pexidartinib prevented cachexia and hypoglycemia in the Ndufs4(KO) mice in a dose-dependent manner (FIGS. 6E, 6F), consistent with the effects of mTOR inhibition and PI3Kγ inhibition (FIGS. 4G-4I).

Abnormally high blood lactate (often defined by the lactate/pyruvate ratio) is a major hallmark of LS and some other forms of MD, and increased lactate is a feature of LS CNS lesions in MRS. Given the benefits of mTOR inhibition, PI3Kγ inhibition, and leukocyte depletion on cachexia and glycemia, experiments were performed to investigate whether hyperlactemia might also be attenuated. While increased blood lactate is generally thought to arise from the primary mitochondrial dysfunction, leukocytes are known to be highly glycolytic, so a role for leukocytes in contributing to increased circulating lactate appeared reasonable.

To probe hyperlactemia in untreated Ndufs4(KO) mice, blood lactate levels were measured at baseline and in response to a glucose bolus in a glucose tolerance test (GTT) paradigm. While clearance of glucose was not significantly altered in the Ndufs4(KO), and blood lactate was not significantly increased in Ndufs4(KO) animals at baseline, exposure of Ndufs4(KO) animals to a glucose bolus resulted in a significant rise in blood lactate in Ndufs4(KO) mice compared to controls. This glucose induced hyperlactemia occurred only in Ndufs4(KO) mice older than P35, the approximate age of CNS symptom onset (FIG. 6G). Pexidartinib treatment prevented this spike in blood lactate (FIG. 6H). Given the clinical relevance of hyperlactemia, and the novelty of this finding, experiments were conducted to further probe the relationship between the mTOR, PI3Kγ, and leukocytes, in mediating hyperlactemia in response to glucose. Animals were next tested by treatment with rapamycin or IPI-549. Both compounds similarly prevented the lactate sequelae, while rapamycin (which is known to have pleiotropic effects on metabolism) also lowered baseline lactate and the lactate production in control animals (FIG. 6H).

Hyperlactemia in response to isoflurane is prevented by pexidartinib. Volatile anesthetics (VAs) have been shown to directly inhibit electron transport chain complex I (ETC CI). Hypersensitivity to VAs is a feature of some forms of MD, and no intervention has yet been shown to attenuate MD VA hypersensitivity. Hypersensitivity to, and toxicity from, VAs is conserved from invertebrates to mammals, including Ndufs4(KO) mice and human LS patients with ETC CI defects. An unrelated pilot study recently found that low-dose isoflurane exposure leads to a blood lactate spike in Ndufs4(KO) mice compared to controls. Given the metabolic findings, experiments were performed to test whether pexidartinib might impact isoflurane induced hyperlactemia. Remarkably, treatment fully suppressed this lactate spike in Ndufs4(KO) animals (FIG. 6I).

In the Ndufs4(KO) mice, hypersensitivity to sedation by VAs occurs at all ages, with some evidence suggesting an increase in sensitivity as CNS disease progresses (Roelofs et al. J Anesth 2014; 28:807-814). To test whether leukocyte depletion might impact VA hypersensitivity, the Minimum Alveolar Concentration (MAC) of isoflurane associated with anesthesia in control and 300 mg/kg/day pexidartinib treated Ndufs4(KO) animals was assessed (see Experimental Methods in Example 1 for details). The experiments focused on P30 animals to assess sensitivity in the absence of overt neurologic dysfunction. Pexidartinib treatment led to a modest, but statistically significant, attenuation of the hypersensitivity to sedation by isoflurane in the Ndufs4(KO) mice (FIG. 6I).

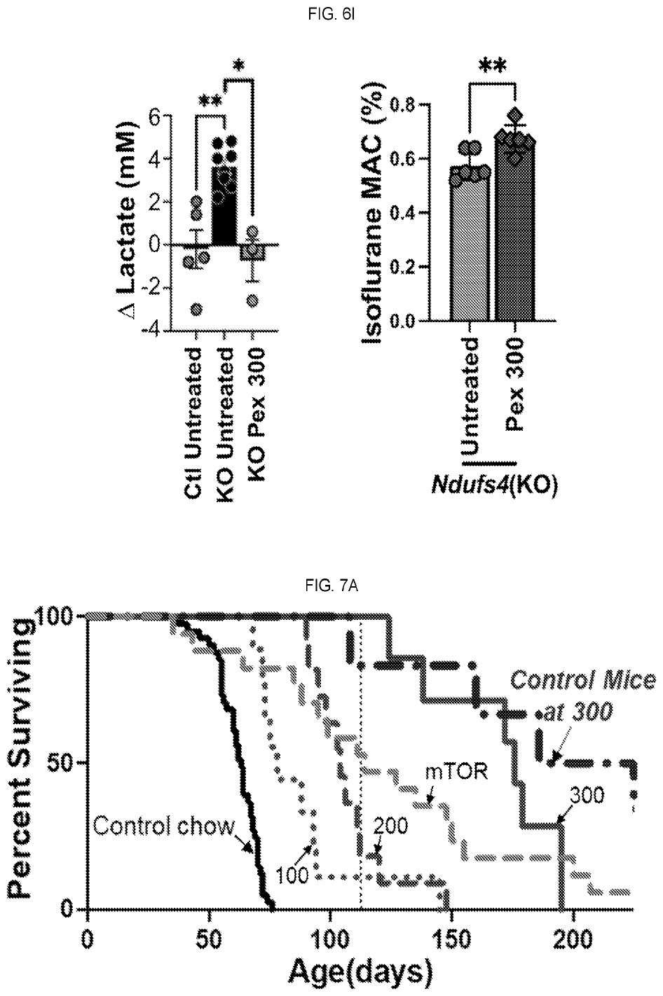

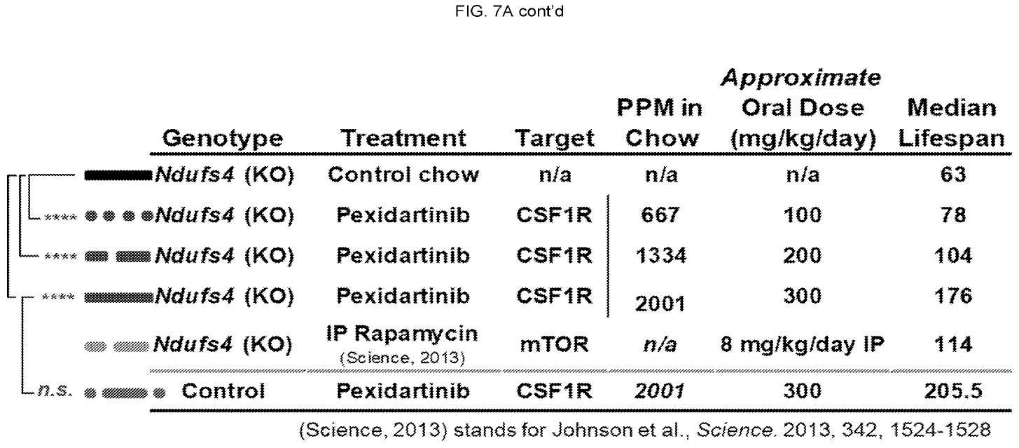

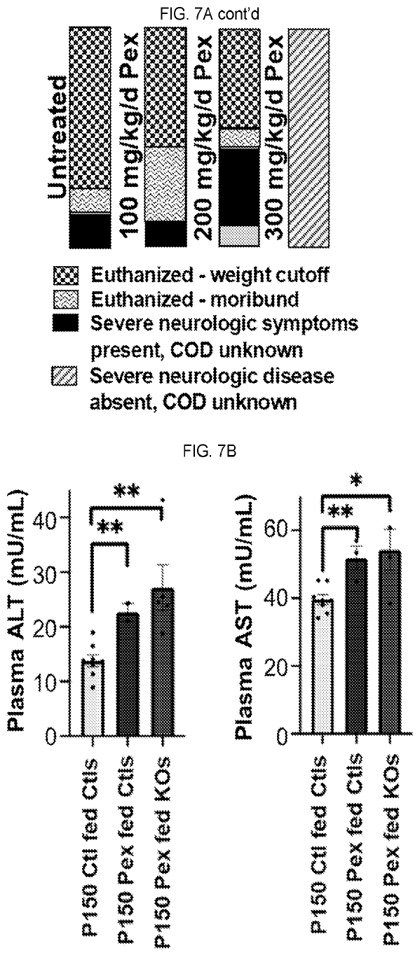

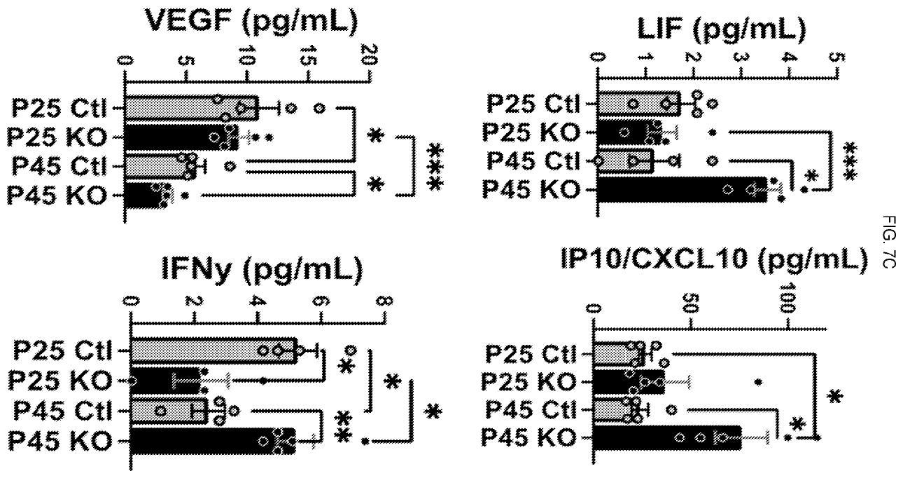

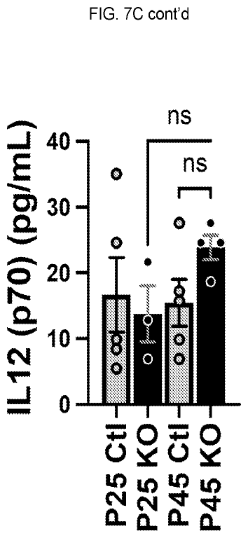

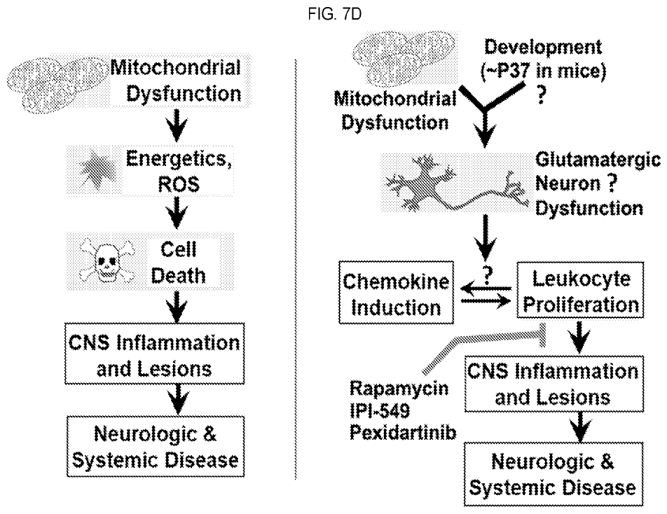

Pexidartinib significantly increases Ndufs4(KO) lifespan, while drug toxicity limits survival in pexidartinib treated animals. Consistent with the rescue of multiple measures of disease, pexidartinib dramatically extended survival of Ndufs4(KO) mice. Survival was extended in a dose-dependent manner from 100-300 mg/kg/day, each of which provided a statistically significant increase compared to untreated Ndufs4(KO) mice. 300 mg/kg/day pexidartinib extended median survival by 300%; critically, the survival curves of control and Ndufs4(KO) animals treated with 300 mg/kg/d pexidartinib overlap, indicating that the drug treatment, rather than underlying mitochondrial disease, is limiting lifespan in this context (FIG. 7A). Consistent with this notion, the majority of high dose pexidartinib animals did not show overt signs of severe neurologic sequelae prior to death; the proximal cause of death was typically unknown in both Ndufs4(KO) and control animals (FIG. 7A).

Pexidartinib is known to cause liver damage, and hepatic function is carefully monitored in patients undergoing treatment with pexidartinib. Accordingly, we tested blood alanine aminotransferase (ALT) and aspartate aminotransferase (AST), markers of hepatic damage, in control and Ndufs4(KO) mice treated with 300 mg/kg/day pexidartinib to assess whether hepatotoxicity may contribute to limiting survival in treated animals. Consistent with this notion, levels of both ALT and AST were significantly elevated in both genotypes as a function of treatment with pexidartinib (FIG. 7B).

Inflammatory chemokines IFNγ and IP-10 are significantly upregulated in Ndufs4(KO) brainstem, but only at ages associated with disease. Taken together, the rapamycin, IPI-549, and pexidartinib data reveal that leukocyte proliferation is a key causal step in the pathogenesis of LS. The specific age of onset of disease in this model, and the postnatal onset of LS in humans, suggests that this leukocyte proliferation is induced by some postnatal event. In addition, the IPI-549 data suggests that leukocyte proliferation is driven at least in part by extracellular signaling, suggesting that some factor or factors may be involved.

To assess whether factors driving leukocyte proliferation is induced in the Ndufs4(KO) model in a manner consistent with the age-specific onset of disease, a targeted cytokine profiling of control and Ndufs4(KO) brainstem from P25 animals was performed, before overt disease is evident, and from P45 animals, early in disease progression. Among the 23 factors in the panel reliably detected above background, four showed significantly altered expression in Ndufs4(KO) mice compared to controls at P45. Interferon gamma (IFNγ), IFNγ-Induced Protein 10 (IP-10/CXCL10), and Leukemia Inhibitory Factor (LIF), an inhibitor of leukocyte differentiation were all increased in the Ndufs4(KO) animals at P45, while VEGF was significantly reduced (FIG. 7C). IL-12 (p70) was also increased (though not reaching statistical significance) in Ndufs4(KO) mice only at P45 (FIG. 7C).

Inflammatory signaling pathways involve complexity beyond the scope of this study, but it is worth noting IL-12 (p70) is produced by activated antigen-presenting cells, drives IFNγ, IP-10, and LIF expression, and inhibits VEGF, providing a possible link between these factors. Significant questions remain beyond the scope of this study, but these findings reveal one potential causal pathway in the activation of neuroinflammation in LS.

Notably, CSF-1 was not elevated in the Ndufs4(KO); while CSF1R inhibition impairs leukocyte proliferation, this intervention strategy is untargeted, impacting all leukocytes. A more targeted therapeutic based on the precise inflammatory insult, once identified, may provide greater benefits with fewer off-target effects.

A causal role for leukocyte proliferation in the pathobiology of LS. The PI3Kγ and CSF1R inhibitor data demonstrate that leukocyte proliferation is a key causal step in the pathogenesis of LS. Targeting leukocyte proliferation prevented microgliosis, rescued astrocytosis, led to a complete prevention of CNS lesions, and rescued CNS inflammation outside of overt lesions. Leukocyte depletion prevented sequelae linked to the overt CNS lesions including impaired respiratory function and behavioral endpoints related to balance and movement. Moreover, leukocyte depletion rescued, or attenuated, sequelae of LS not previously attributed to these degenerative lesions—hypoglycemia, cachexia, hyperlactemia in response to a glucose bolus, hyperlactemia in response to anesthesia, seizures, and anesthesia sensitivity. The relative contribution of central versus peripheral leukocytes to these disease features remains to be determined, but the data suggests that inflammatory cells mediate at least a portion of non-CNS pathologies of LS.

As mentioned, it is particularly notable that human disease often first appears after a viral infection or fever of unknown origin. IFNγ is a component of the innate immune response to viruses, and the data suggests that IFNγ production provides a mechanistic link between viral infection and disease onset in this disease. Why IFNγ and related factors are upregulated at a specific age in mice, and whether these same factors are in fact increased in human LS patients, remains to be determined. Given the role of IFNγ in responding to intracellular pathogens and the established role of a specific subset of neurons in driving disease, it is tempting to hypothesize, without being bound by any one hypothesis, that mitochondrial defects in these neurons interacts with neurodevelopment in a manner which leads to sensing of some mitochondrial component as ‘foreign’ by the innate immune machinery.

Further study will be needed to precisely define the roles of peripheral versus CNS leukocyte populations in the pathogenesis of LS.

Determining the mechanisms underlying the benefits of rapamycin in the Ndufs4(KO) model has been an active area of investigation since the effects of the drug were published in 2013 (Johnson et al. Front Genet. 2015; 6:247; Johnson et al. Science. 2013; 342(6165):1524-1528; Johnson, SC. Translational Medicine. A target for pharmacological intervention in an untreatable human disease. Science 2014; 346:1192). The data presented here reveal that the benefits of mTOR inhibition in the Ndufs4(KO) result primarily from their effects on leukocyte, including microglia, proliferation. Furthermore, while PI3Kγ inhibition recapitulates mTOR inhibition, the data presented here demonstrate that direct pharmacologic targeting of leukocyte proliferation significantly outperforms both PI3Kγ and mTOR inhibition.

While leukocyte proliferation appears to be the primary mechanism underlying the benefits of mTOR inhibition in LS, it is important to note that mTOR inhibitors have shown beneficial effects in a variety of mitochondrial disease models, including in cultured cells. mTOR inhibition is highly pleiotropic, and it is very likely that other processes regulated by mTOR, such as metabolism, mediate beneficial effects of targeting mTOR in other forms of mitochondrial dysfunction. That said, it is also possible that previously unappreciated immune dysfunction may play a role in the presentation of other forms of MD, a possibility which may warrant further attention.

A new model for the pathogenesis of LS involving mitochondria-induced inflammation. Onset of symptoms in the Ndufs4(KO) occurs at P37, reminiscent of human LS where patients are often overtly healthy at birth, and this study shows that pro-inflammatory chemokines are increased in mice only after the age of symptom onset. Prior studies have demonstrated that neuron specific (nestin-Cre) or glutamatergic neuron specific (VGIut2-Cre) deletion of Ndufs4 results in a near-complete recapitulation of the disease seen in the whole-body knockout including CNS lesions, cachexia, metabolic dysfunction, behavioral deficits, and reduced survival. In contrast, deletion of Ndufs4 in only GABAergic neurons drives seizures but no other overt phenotype, while mice with cholinergic loss of Ndufs4 have no overt disease. Considering the data here in the context of this work, without being bound by any one theory, a model can be assembled for the pathogenesis of disease in the Ndufs4(KO) (FIG. 7D): mitochondrial dysfunction interacts with some developmental change in glutamatergic neurons at P37 which leads to the induction of IFNγ and related chemokines. These chemokines drive leukocyte proliferation, leading to tissue degeneration, astrocytosis, CNS dysfunction, and systemic symptoms.

The present data provide new insight into the biggest mysteries surrounding the complex pathogenesis of LS. In particular, 1) the relative sparing of tissues that have high energy requirements, such as the lack of any overt phenotype in cardiac muscle specific Ndufs4(KO) mice, 2) the inability of antioxidants, caloric supplementation, or alternate energetic substrates to markedly attenuate disease, and 3) the curious post-natal onset. Without being bound by any one theory, the enigmatic specificity of CNS lesion sites may also be explained by a glutamatergic neuron/development/inflammation interaction that is region specific.

Viral infection and fever have been reported to occur in conjunction with symptom onset or neurodegenerative events in MDs of childhood and adulthood, and in certain other forms of acute focal necrotizing encephalopathy in children (Edmonds et al. Arch Otolaryngol Head Neck Surg 2002; 128:355-362; Lee et al. J Korean Med Sci 2019; 34:e143; Niyazov et al. Mol Syndromol 2016; 7:122-137; Porta et al. J Pediatr Endocrinol Metab 2021; 34:261-266; Wang and Huang. Chang Gung Med J 2001; 24:1-10; Wei and Wang. Neurol Sci 2018; 39:2225-2228; Wu et al. Zhonghua Min Guo Xiao Er Ke Yi Xue Hui Za Zhi 1993; 34:301-307). This link in MD has been attributed, by some, to energetic stress resulting from the requirements of mobilizing an immune response (Niyazov et al. Mol Syndromol 2016; 7:122-137). The present data suggests intracellular pro-inflammatory signaling may play a role in at least some of these settings.

The present data identify a potent and effective target for therapy, but the benefits of pexidartinib in the preclinical model were limited by toxicity, as determined by the effects on control animals. Improved CNS targeting to lower necessary dosing, identification of more specific targets for intervention (such as inhibitors of the receptor for IP10), testing of combinatorial therapies, and/or chemical modifications to current inhibitors to lower toxicity, will lead to robust small molecule therapeutic options.

(xii) Closing Paragraphs. Unless otherwise indicated, the practice of the present disclosure can employ conventional techniques of chemistry, organic chemistry, biochemistry, analytical chemistry, and physical chemistry. These methods are described in the following publications. See, e.g., Harcourt, et al., Holt McDougal Modern Chemistry: Student Edition (2018); J. Karty, Organic Chemistry Principles and Mechanisms (2014); Nelson, et al., Lehninger Principles of Biochemistry 5th edition (2008); Skoog, et al., Fundamentals of Analytical Chemistry (8th Edition); Atkins, et al., Atkins' Physical Chemistry (11th Edition).

Each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to: treat an encephalopathy, reduce neuroinflammation, reduce leukocyte proliferation, reduce neurologic symptoms, improve respiratory function, reduce frequency of seizures, reduce cachexia, reduce hypoglycemia, reduce hyperlactemia, and/or reduce sensitivity to volatile anesthetics in a subject in need thereof with an inhibitor and/or immunosuppressant drug as described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method of treating an encephalopathy in a subject in need thereof comprising administering a composition to the subject, wherein the composition comprises a therapeutically effective amount of an inhibitor upstream of mTOR in the CSF1 pathway of neuroinflammation, thereby treating the encephalopathy.

2. The method of claim 1, wherein the encephalopathy comprises a genetic encephalopathy or an environmental encephalopathy.

3. The method of claim 1, wherein the encephalopathy comprises a mitochondrial encephalopathy.

4. The method of claim 1, wherein the encephalopathy comprises Leigh Syndrome or Wernicke encephalopathy.

5. The method of claim 1, wherein the inhibitor upstream of mTOR in the CSF1 pathway of neuroinflammation binds the CSF1 receptor and interferes with binding by the natural CSF-1 ligand.

6. The method of claim 5, wherein the inhibitor upstream of mTOR in the CSF1 pathway of neuroinflammation comprises PLX 5622.

7. The method of claim 5, wherein the inhibitor upstream of mTOR in the CSF1 pathway of neuroinflammation comprises Pexidartinib.

8. The method of claim 1, wherein the inhibitor upstream of mTOR in the CSF1 pathway of neuroinflammation inhibits the P110γ microglia specific catalytic subunit of PI3K.

9. The method of claim 7, wherein the inhibitor upstream of mTOR in the CSF1 pathway of neuroinflammation comprises IPI-549.

10. A method of treating an encephalopathy in a subject in need thereof comprising administering a composition to the subject, wherein the composition comprises a therapeutically effective amount of an inhibitor upstream of mTOR in the CSF1 pathway of neuroinflammation;
an inhibitor of chemokine (C—X—C motif) receptor 3 (CXCR3); and/or
an immunosuppressant drug,
thereby treating the encephalopathy.

11. The method of claim 10, wherein the encephalopathy comprises a genetic encephalopathy or an environmental encephalopathy.

12. The method of claim 10, wherein the encephalopathy comprises a mitochondrial encephalopathy.

13. The method of claim 10, wherein the encephalopathy comprises Leigh Syndrome or Wernicke encephalopathy.

14. The method of claim 10, wherein the inhibitor upstream of mTOR in the CSF1 pathway of neuroinflammation binds the CSF1 receptor and interferes with binding by the natural CSF-1 ligand.

15. The method of claim 14, wherein the inhibitor upstream of mTOR in the CSF1 pathway of neuroinflammation comprises PLX 5622.

16. The method of claim 14, wherein the inhibitor upstream of mTOR in the CSF1 pathway of neuroinflammation comprises Pexidartinib.

17. The method of claim 10, wherein the inhibitor upstream of mTOR in the CSF1 pathway of neuroinflammation inhibits the P110γ microglia specific catalytic subunit of PI3K.

18. The method of claim 17, wherein the inhibitor upstream of mTOR in the CSF1 pathway of neuroinflammation comprises IPI-549.

19. The method of claim 10, wherein the inhibitor of CXCR3 binds CXCR3 and interferes with binding by a natural ligand of CXCR3.

20. The method of claim 19, wherein the natural ligand of CXCR3 is interferon γ-inducible 10 kD Protein (IP-10).

21. The method of claim 10, wherein the inhibitor of CXCR3 comprises: AMG487; TAK-779; SCH 546738; NBI-74330; and/or PS372424.

22. The method of claim 10, wherein the immunosuppressant drug comprises a corticosteroid, a Janus kinase inhibitor, a calcineurin inhibitor, an mTOR inhibitor, an inosine monophosphate dehydrogenase (IMDH) inhibitor, a biologic, or a combination thereof.

23. The method of claim 22, wherein the corticosteroid comprises prednisone, budesonide, prednisolone, dexamethasone, or a combination thereof.

24. The method of claim 22, wherein the Janus kinase inhibitor comprises tofacitinib.

25. The method of claim 22, wherein the calcineurin inhibitor comprises cyclosporine, tacrolimus, or a combination thereof.

26. The method of claim 22, wherein the mTOR inhibitor comprises sirolimus, everolimus, or a combination thereof.

27. The method of claim 22, wherein the IMDH inhibitor comprises azathioprine, leflunomide, mycophenolate, or a combination thereof.

28. The method of claim 22, wherein the biologic comprises abatacept, adalimumab, anakinra, certolizumab, etanercept, golimumab, infliximab, ixekizumab, natalizumab, rituximab, secukinumab, tocilizumab, ustekinumab, and vedolizumab, basiliximab, daclizumab, or a combination thereof.

29. The method of claim 10, further comprising recommending that the subject ingest a ketogenic diet.

30. The method of claim 10, further comprising administering to the subject a composition comprising a secondary active ingredient.

31. The method of claim 30, wherein the secondary active ingredient is within a ketogenic diet.

32. The method of claim 30, wherein the secondary active ingredient comprises Coenzyme Q, idebenone, acetylcarnitine, palmitoylcarnitine, carnitine, quercetine, mangosteen, acai, uridine, N-acetyl cysteine, a polyphenol, Vitamin A, Vitamin C, lutein, beta-carotene, lycopene, glutathione, a fatty acid, lipoic acid, a Vitamin B complex, Vitamin B1, Vitamin B2, Vitamin B3, Vitamin B5, Vitamin B6, Vitamin B7, Vitamin B9, Vitamin B12, inositol, 4-aminobenzoic acid, folinic acid, and/or Vitamin E.

33. The method of claim 10, wherein the subject is a mouse.

34. The method of claim 10, wherein the subject is a human.

35. The method of claim 10, wherein the subject is a human under the age of 7 years old.

36. The method of claim 10, wherein the subject is a human under the age of 3 years old.

37. The method of claim 10, wherein the subject is a human under the age of 1 year old.

38. A pharmaceutical composition comprising:
an inhibitor upstream of mTOR in the CSF1 pathway of neuroinflammation;
an inhibitor of chemokine (C—X—C motif) receptor 3 (CXCR3);
an immunosuppressant drug; and/or
a secondary active ingredient.

39. The pharmaceutical composition of claim 38, wherein the inhibitor upstream of mTOR in the CSF1 pathway of neuroinflammation comprises PLX 5622, Pexidartinib, or IPI-549.

40. The pharmaceutical composition of claim 38, wherein the inhibitor upstream of mTOR in the CSF1 pathway of neuroinflammation comprises a functional derivative of PLX 5622, Pexidartinib, or IPI-549.

41. The pharmaceutical composition of claim 38, wherein the inhibitor of CXCR3 comprises: AMG487; TAK-779; SCH 546738; NBI-74330; and/or PS372424.

42. The pharmaceutical composition of claim 38, wherein the immunosuppressant drug comprises a corticosteroid, a Janus kinase inhibitor, a calcineurin inhibitor, an mTOR inhibitor, an inosine monophosphate dehydrogenase (IMDH) inhibitor, a biologic, or a combination thereof.

43. The pharmaceutical composition of claim 42, wherein the corticosteroid comprises prednisone, budesonide, prednisolone, dexamethasone, or a combination thereof.

44. The pharmaceutical composition of claim 42, wherein the Janus kinase inhibitor comprises tofacitinib.

45. The pharmaceutical composition of claim 42, wherein the calcineurin inhibitor comprises cyclosporine, tacrolimus, or a combination thereof.

46. The pharmaceutical composition of claim 42, wherein the mTOR inhibitor comprises sirolimus, everolimus, or a combination thereof.

47. The pharmaceutical composition of claim 42, wherein the IMDH inhibitor comprises azathioprine, leflunomide, mycophenolate, or a combination thereof.

48. The pharmaceutical composition of claim 42, wherein the biologic comprises abatacept, adalimumab, anakinra, certolizumab, etanercept, golimumab, infliximab, ixekizumab, natalizumab, rituximab, secukinumab, tocilizumab, ustekinumab, and vedolizumab, basiliximab, daclizumab, or a combination thereof.

49. The pharmaceutical composition of claim 38, wherein the secondary active ingredient is within a ketogenic diet.

50. The pharmaceutical composition of claim 38, wherein the secondary active ingredient comprises Coenzyme Q, idebenone, acetylcarnitine, palmitoylcarnitine, carnitine, quercetine, mangosteen, acai, uridine, N-acetyl cysteine, a polyphenol, Vitamin A, Vitamin C, lutein, beta-carotene, lycopene, glutathione, a fatty acid, lipoic acid, a Vitamin B complex, Vitamin B1, Vitamin B2, Vitamin B3, Vitamin B5, Vitamin B6, Vitamin B7, Vitamin B9, Vitamin B12, inositol, 4-aminobenzoic acid, folinic acid, and/or Vitamin E.

51. A kit comprising:
an inhibitor upstream of mTOR in the CSF1 pathway of neuroinflammation;
an inhibitor of chemokine (C—X—C motif) receptor 3 (CXCR3);
an immunosuppressant drug; and/or
a secondary active ingredient.

52. The kit of claim 51, wherein the inhibitor upstream of mTOR in the CSF1 pathway of neuroinflammation comprises PLX 5622, Pexidartinib, or IPI-549.

53. The kit of claim 51, wherein the inhibitor upstream of mTOR in the CSF1 pathway of neuroinflammation comprises a functional derivative of PLX 5622, Pexidartinib, or IPI-549.

54. The kit of claim 51, wherein the inhibitor of CXCR3 comprises: AMG487; TAK-779; SCH 546738; NBI-74330; and/or PS372424.

55. The kit of claim 51, wherein the immunosuppressant drug comprises a corticosteroid, a Janus kinase inhibitor, a calcineurin inhibitor, an mTOR inhibitor, an inosine monophosphate dehydrogenase (IMDH) inhibitor, a biologic, or a combination thereof.

56. The kit of claim 55, wherein the corticosteroid comprises prednisone, budesonide, prednisolone, dexamethasone, or a combination thereof.

57. The kit of claim 55, wherein the Janus kinase inhibitor comprises tofacitinib.

58. The kit of claim 55, wherein the calcineurin inhibitor comprises cyclosporine, tacrolimus, or a combination thereof.

59. The kit of claim 55, wherein the mTOR inhibitor comprises sirolimus, everolimus, or a combination thereof.

60. The kit of claim 55, wherein the IMDH inhibitor comprises azathioprine, leflunomide, mycophenolate, or a combination thereof.

61. The kit of claim 55, wherein the biologic comprises abatacept, adalimumab, anakinra, certolizumab, etanercept, golimumab, infliximab, ixekizumab, natalizumab, rituximab, secukinumab, tocilizumab, ustekinumab, and vedolizumab, basiliximab, daclizumab, or a combination thereof.

62. The kit of claim 51, wherein the secondary active ingredient is within a ketogenic diet.

63. The kit of claim 51, wherein the secondary active ingredient comprises Coenzyme Q, idebenone, acetylcarnitine, palmitoylcarnitine, carnitine, quercetine, mangosteen, acai, uridine, N-acetyl cysteine, a polyphenol, Vitamin A, Vitamin C, lutein, beta-carotene, lycopene, glutathione, a fatty acid, lipoic acid, a Vitamin B complex, Vitamin B1, Vitamin B2, Vitamin B3, Vitamin B5, Vitamin B6, Vitamin B7, Vitamin B9, Vitamin B12, inositol, 4-aminobenzoic acid, folinic acid, and/or Vitamin E.

64. A method of reducing glial cell activation in a subject in need thereof comprising administering a composition to the subject, wherein the composition comprises a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug,
thereby reducing the glial cell activation, as compared to glial cell activation when the subject is not administered the composition.

65. A method of reducing neuroinflammation in a subject in need thereof comprising administering a composition to the subject, wherein the composition comprises a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug,
thereby reducing the neuroinflammation, as compared to neuroinflammation in the subject when the subject is not administered the composition.

66. A method of reducing leukocyte proliferation in a subject in need thereof comprising administering a composition to the subject, wherein the composition comprises a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug,
thereby reducing the leukocyte proliferation in the subject, as compared to leukocyte proliferation in the subject when the subject is not administered the composition.

67. A method of improving respiratory function in a subject in need thereof comprising administering a composition to the subject, wherein the composition comprises a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug,
thereby improving the respiratory function in the subject, as compared to respiratory function in the subject when the subject is not administered the composition.

68. A method of reducing frequency of seizures in a subject in need thereof comprising administering a composition to the subject, wherein the composition comprises a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug,
thereby reducing the frequency of seizures in the subject, as compared to frequency of seizures in the subject when the subject is not administered the composition.

69. A method of reducing hypoglycemia in a subject in need thereof comprising administering a composition to the subject, wherein the composition comprises a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug,
thereby reducing the hypoglycemia in the subject, as compared to hypoglycemia in the subject when the subject is not administered the composition.

70. A method of reducing hyperlactemia in a subject in need thereof comprising administering a composition to the subject, wherein the composition comprises a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug,
thereby reducing the hyperlactemia in the subject, as compared to hyperlactemia in the subject when the subject is not administered the composition.

71. A method of reducing sensitivity to a volatile anesthetic in a subject comprising administering a composition to the subject, wherein the composition comprises a therapeutically effective amount of
an inhibitor upstream of mTOR in the CSF1 pathway of neuroinflammation,
an inhibitor of chemokine (C—X—C motif) receptor 3 (CXCR3); and/or
an immunosuppressant drug,
thereby reducing the sensitivity to the volatile anesthetic in the subject, as compared to sensitivity to the volatile anesthetic in the subject not administered the composition.

72. The method of claim 71, wherein the volatile anesthetic comprises nitrous oxide, isoflurane, desflurane, halothane, and sevoflurane.
